SEQUENCE LISTING (74 sequences)

SEQ ID NO: 1 - DNA, 216 bp, Ophelimus maskelli
atgtctaagaaggatagcaacaaaatgctggttcaccagttatcatccgtattaccagacgatcgaccaatcacagcaataaatattatcgaggatttggataagtgtccacctaatttcactgttgtatcaaggacctatgacttagattcagatgcagacttatggcgagaaagtggactatttatcaaaaagaaatccagatacatatgtttc

SEQ ID NO: 2 - DNA, 100 bp, Ophelimus maskelli
gtactaagtccaagtgccagaataaggccaacgcgaccagcgccaaggccaccgtctaataaatcgccattggttcattctgtgccagcttatggaacat

SEQ ID NO: 3 - DNA, 675 bp, Ophelimus maskelli
atgttgcgttcccttttagcaaaccatacctttcaacaaggtctcaatttggtggcacgtaacaatgctgctatcttgcgatgtcagaatccactcttggagcatgctaaaacaatggcaacacaatcggtaaatcaaagttctggaagttcagctccagcaccagcaaaaaaagaaccttacagtccattccaaaacacaagtaacatggttgaatatgccttagctagactagacgatctgttaaattggggaagaaaaggttcaatgtggccactgacgttcgggctggcatgttgtgctgtggaaatgatgcacattgctgctccacgatacgatatggacagatttggtgtagttttccgtgcttctcctaggcatgctgatgttatcatagtagctggtacagtgacaaataaaatggcaccggcgttgagaaaagtatacgatcagatgcctgatcccaaatgggtcatctcaatgggtagttgtgcaaatggtggaggctattatcactacagttactcagttgtaaggggttgcgatagaattatacctgtggacatttatgtacctggctgtcctccaacagctgaagcattactttatggagttttacagctgcagaaaaaggtgaaacgtatggagactgcccaagtgtggtacagaaaatag

SEQ ID NO: 4 - DNA, 100 bp, Ophelimus maskelli
tcccttttagcaaaccatacctttcaacaaggtctcaatttggtggcacgtaacaatgctgctatcttgcgatgtcagaatccactcttggagcatgcta

SEQ ID NO: 5 - DNA, 639 bp, Ophelimus maskelli
atgtctattgcacaagatcgtcctgaactttttgaagaagtaaagctgtacaaaaatgctagggaacgtgaaaaatatgataaccaggccgatttatatgctgttgttaatactttacagcatttagagaaagcttatatcagagactgtgttacgcccaaagaatatacagctgcatgcagcaaattattagttcaatatagagctgcttttaaacaggtccagagcgatcaatttccaacaattgatgcttttgccagagcatttagacttgattgtccagcagcacttgaacgaattaaagaagatagacctatcaccattaaagatgacaaaggcaatacttctaaatgtattgctgacattgtttcactcttcataacattaatggataagttacgacttgaaattaaagctatggatcaacttcatccagatcttagagatttgatggacactatgaacaggcttagtatattgccaagcgattttgatggaaagcaaaaagttgcagaatggcttcagaccctaaataatatgtcagcatctgatgaactatcggatactcaagttagacaactgatattcgatttagaaacatcttacaatgcttttaatagagttttacataactcgtaa

SEQ ID NO: 6 - DNA, 100 bp, Ophelimus maskelli
aatgtattgctgacattgtttcactcttcataacattaatggataagttacgacttgaaattaaagctatggatcaacttcatccagatcttagagattt

SEQ ID NO: 7 - DNA, 720 bp, Ophelimus maskelli
atgttatcgcgaatattccttggggaaaatgaaaatgccgcagtcaagagaaaacgtcagatcgacacgctgaaaattttcaacgacaatgttctggagctgcaagaagatgtcgattatcaagtgcactttgatgcaggaggcaaaagaatggctattcaggtctcgctatctccagactttccactggaaaagccagtacttagagtatcacctccgataaaacataaatggtgcaatgaacacagtgaaatcactagtgctccaggattattaaattttacagtacacagtgatctgggtcgtgttgttcaagccattatacgagaatttagtaaaaatccaccacagttgttagaagaaagttctccagtatcaaatctagcgcttggagatttagctggaagaacgtcaccatcttcttacatgtggaaaccatgtgatcttgcctcaggatcttataactcatactacaatccttcattccagcagttttcttcgtcaaatgctacacctagcatttataactataattacacaaatgctagcaatacatatttgggcaattcgctaagctccgttcagtttaatacaacaaatgctcaatcacagtactcatcgagtcctagcgcaaatgcttacacaaatttacattatgctaatgccaacatgggaccatcgatgcagaatcaagtgcaatataaaagtaataaagtaacc

SEQ ID NO: 8 - DNA, 100 bp, Ophelimus maskelli
gaaagttctccagtatcaaatctagcgcttggagatttagctggaagaacgtcaccatcttcttacatgtggaaaccatgtgatcttgcctcaggatctt

SEQ ID NO: 9 - DNA, 336 bp, Ophelimus maskelli
atgtttaaaccaccacaagaaccaaatatagctgctgctattgcaccaatttcagatttaagtacggatgcattgaaagagttgttgaatgatgatgaaaagtttgaagagattatcaaagataatcaacagttactagagttagaatcagaaaaagaagagattatggtacggaatcgatcgctggcagaatttaatttatcaaaagaaccagaattggaggaatgtaaaaggaaaatacaatcacttagcgaagaaggcaataatctttgcgctagtgtgcaagccaaacttgaacaaataacaaataaagctggaactatgacagttgatact

SEQ ID NO: 10 - DNA, 100 bp, Ophelimus maskelli
atgaaaagtttgaagagattatcaaagataatcaacagttactagagttagaatcagaaaaagaagagattatggtacggaatcgatcgctggcagaatt

SEQ ID NO: 11 - DNA, 366 bp, Ophelimus maskelli
atgaggagaaaacctggagttggagcaattcataaacaaaagtatgaacaagaaaaatataaagataaaggaacggagcttcaagaaaatcaattcgagcagatgacgaagcaactggaaacttttcgaataaatttagaagactttgcttcaaaacacaagaatgaaataaagaaaaatgcgcacttcaggaagcaatttacagatatgtgtgcctcaattggtgtagatccattagcatcaggaaaaggattttggtctgttcttggtataggagacttttattatgaaattgcagtacaaattgtagaagtttgcttggcgacaaactataaaaatggaggtttgatatctttagacgaatta

SEQ ID NO: 12 - DNA, 100 bp, Ophelimus maskelli
agcaactggaaacttttcgaataaatttagaagactttgcttcaaaacacaagaatgaaataaagaaaaatgcgcacttcaggaagcaatttacagatat

SEQ ID NO: 13 - DNA, 501 bp, Ophelimus maskelli
atgactgaaattgactggccctggcaatatagtttccacctttttttcacacttcaaccccatacagaaacaagagtaaagcagatagaggcatggaaaactttagttttgaattacttcaggatagctaaacaagcgattcttgatgttcgagaaatccatagcactccattgtttaacaacacttctattgacagaaagctaccactagaagtcgtatctataatagtagaagaattagcaaaatcaggtaatgcaatacctttggataaattgaaacaacgatggattatatcgtggcacacattagaagaatgggctgatactatatactcgtgggcacaagcaaatggatttattgggtctgtttgtacactttatgaactaacacaaggagagaatacagtggatgaagagttttatggtttggacaacgaattacttataagatctctaaggacactggaagcttctaaaaaggcagaactaattatgtttgac

SEQ ID NO: 14 - DNA, 100 bp, Ophelimus maskelli
ttcttgatgttcgagaaatccatagcactccattgtttaacaacacttctattgacagaaagctaccactagaagtcgtatctataatagtagaagaatt

SEQ ID NO: 15 - DNA, 441 bp, Ophelimus maskelli
tttaaatcttatctcatgagtcttggtattgatgatccagttactagagaggcctataaaagtagtaacgaatatttcaaacaacttgccaggcagttagctgaaattttggatgaaccaattagggaggtaggtggaatgatggctttaactgatgtatactgtcgcgtcaatagagcaagaggtttggagcttctctcacctgaagatttacttcatgcgtgtagacaattagctccattaaatctgccaatagtattaaggatctttgacagcggtgttatggttctacaatctagagcacataacgattatgaaattgctgaagctgtagctcaattgatcaagagcagaggctcgataacagcaacggaactggcacaatctgaaggtatatcaatggttttagcttgcgaaaggcttctaatgactgaaaaatac

SEQ ID NO: 16 - DNA, 100 bp, Ophelimus maskelli
aatctagagcacataacgattatgaaattgctgaagctgtagctcaattgatcaagagcagaggctcgataacagcaacggaactggcacaatctgaagg

SEQ ID NO: 17 - DNA, 447 bp, Ophelimus maskelli
atggctaaagatggacaaatggatgcggttaaaattatggcaaaagatcttgtgaggactaggcgctatgttaagaaattcatgttaatgaaagcgaatattcaagctgtgtccctcaaaatacaaactcttaaatctcaaaacacaatggctcaagcaatgaaaggtgtcacaaaagctatgcaaaatatgaacaagcaactcaacttacctcaaatccaaaagattttgcaagagtttgagaaacaatccgaagtcatggacatgaaagaagaaataatgaacgatgcgattgatgatgcaatggaagatgagggtgatgaagaggaaagtgacgcaattgtatcccaagttctggacgaactaggcctccaactcaccgaccaattgtccggtctacctcaagcctcagggtctctgagtatagcaggctccaagcaaccagta

SEQ ID NO: 18 - DNA, 100 bp, Ophelimus maskelli
gtgacgcaattgtatcccaagttctggacgaactaggcctccaactcaccgaccaattgtccggtctacctcaagcctcagggtctctgagtatagcagg

SEQ ID NO: 19 - DNA, 312 bp, Ophelimus maskelli
ttagagaatttagagcgaatggtacatgacttggagtttgcacaagttgaagctaaagttatcgatggccttaaagtcggcaatgatgccctcaaaaagttacatgcaatattatcaattgatgaaattgaaaaagtcatggatgaaaccagagaaggtgtagagaaacaacaagaaattgatgcacttttgtctggagcactgagtgatgaagatgaaaatgatgttgaaactgaacttaatgctcttatctcgcaagaagccgatgtaataaccacgcctgaagtaccagaagatgttccaatgcctgac

SEQ ID NO: 20 - DNA, 100 bp, Ophelimus maskelli
agatgaaaatgatgttgaaactgaacttaatgctcttatctcgcaagaagccgatgtaataaccacgcctgaagtaccagaagatgttccaatgcctgac

SEQ ID NO: 21 - DNA, 552 bp, Ophelimus maskelli
atccagagagaagaagaaaaagtgaaaagatcattaaaagatgcagccaaaaaaggtgataaagatgtatgtaaagtattggcaaaagaagttattcgagccagaactgcatgtaataaactgcatacatcgaaagcacatcttaattcagttaccttacaaatgaaaaatcagctggcaacaataagagtagctggatctctttcaaaatcaacagaagttatgcaagcaatgcaagctctcgtaaaagttccagaagttgcagcaaccatgagagaactttcaaaagaaatgatgaaagctgggataattgaagaaatgatggatgaaaccttggattctattgaagattctgaagaattggaagatgcagcagatgaagaagttgataaaattttatgggaagtcactgctggacagatgggaagagcgcctgatgttgtcactgatacacctggagcatctacgtctaaagaagaagaagttgaagaagtcacggatgacaaagaattagaagaaatgaaaaataggttacaaagccttcgcagctag

SEQ ID NO: 22 - DNA, 100 bp, Ophelimus maskelli
agaaaaagtgaaaagatcattaaaagatgcagccaaaaaaggtgataaagatgtatgtaaagtattggcaaaagaagttattcgagccagaactgcatgt

SEQ ID NO: 23 - DNA, 663 bp, Ophelimus maskelli
atgagcttcttcacgaaggtcttcggcgggaagaaggaggcggctgctccgacgacctcggaggccatacagaaactacgcgagacggaggagatgctcatcaagaagcaagacttcctcgagggcaagatcgagcaggaaattcagcaggctaggaaatacggaactaaaaacaaacgagctgctatccaagcattaaaaaagaagaaacgctatgagaaacaactgcaacaaatcgatggcacattatccacaattgagatgcagagagaggcactcgaaagcgccaacacgaatactgctgttctgactacgatgaagaatgcagcagatgctttgaaagctgctcatcaacacatggatgttgatcaagtccatgatatgatggatgacattgctgaacaacaagatgtggcaaaggaaatttcagatgctatctctaatccagtagcattcggccaagacatagatgaagatgaacttgagaaggaactagaggaattagaacaagaagaattagataaagaattgttgggtataaagactacagacgaattaccgtcagttcctgctacatcattgccagctgtaccagaaaagaaaacaagagcaaaagcagaagatgatgatgatttgagagaactggaacaatgggcatcgtaa

SEQ ID NO: 24 - DNA, 100 bp, Ophelimus maskelli
atgagcttcttcacgaaggtcttcggcgggaagaaggaggcggctgctccgacgacctcggaggccatacagaaactacgcgagacggaggagatgctca

SEQ ID NO: 25 - DNA, 993 bp, Leptocybe invasa
atgtcgaagaaggatggtaacaaaatgctggttcaccagttatcgtctgttctaccggatgataggcctatcacagcaataaatatcatagaagacttggacaagtgtccaccaaacttcactgtggtatcaagaacatatgatttggattcagatgcagacttgtggcgagagagtgggctgtttattaagaaaaagtcaagatacatatgtttttcaaagactgaaggattgccacattctgttatagaaaaaatttcaattcttgatgagagagaaccacctccagagggattcagtatgatttctcatactgtagactcaatgcaaaaagcatggcgtaaaaggcaactgtgttacaaaattagaaataaggacttgtgcacatgtgcagtatcggacataattatctgtagtaggcttggtaaaggttccaaactggctccagatgaatttacctacgctggtgtcataaacggtgtttacctttgctataaaacagttccactactagcgggttcccacacagtacagccatatgcgaacatagaattattcaaagcggcctcgccaacgctgtcgaacggcggcagcacgccacacaggccagcaccggaacgcccgccaaagccaaagttcggggccaagccgacgaatggactgtatccgcaaataagcccgtctaacgcgggaaagtcggaggaaagccaagaccaagactacgagatattgagcccaagcgcgagaataaggccaacgcgaccagcgccaaggccgccatccaacaaatcacctctcgttaattcggtgccatcgtacggaacactgcctggctcgtcggatctcgatggagttccttttattcttaatccgcttctcactagtggcacgtcgctttcgttaaataagcaattccctgtcattaaaatccgaactcagaaagaactggataaagagtattcctacgattttcgaacggagcgagaaacttga

SEQ ID NO: 26 - DNA, 100 bp, Leptocybe invasa
ttcaaagcggcctcgccaacgctgtcgaacggcggcagcacgccacacaggccagcaccggaacgcccgccaaagccaaagttcggggccaagccgacga

SEQ ID NO: 27 - DNA, 666 bp, Leptocybe invasa
atgttgcgttcccttttgactaatcatactttccagcaaggcttgaatttagtggcgagaaataatgtcactattttgggaactcagagtccgctgctggagcagacaaaaaatttagcaacacaaccagttgaaagctcagcaccagcagcaactccagcaaaagaaccttacagcccctttcagaataagagtagcatggttgaatatgcactggctagacttgacgatttgttgaattggggcagaaaaggttctatttggccattgacatttggtttggcttgctgtgctgtggaaatgatgcacattgctgcgccacgttatgatatggacaggtttggtgttgtgtttcgtgcctcgcctaggcatgctgatgtaattattgtagctggtacagtgacaaataagatggctcctgcattgaggaaagtttacgatcaaatgccagaccccaaatgggtgatttctatgggcagctgtgccaatggtggaggctactatcattatagttactctgttgtaaggggctgcgacaggatcatacctgtggacatttatgtacctggctgtcctcccaccgcagaggcactcctttatgggattttgcagttgcagaaaaaagttaagcgaatggagactgcacaagtgtggtatagaaaataa

SEQ ID NO: 28 - DNA, 100 bp, Leptocybe invasa
tcccttttgactaatcatactttccagcaaggcttgaatttagtggcgagaaataatgtcactattttgggaactcagagtccgctgctggagcagacaa

SEQ ID NO: 29 - DNA, 630 bp, Leptocybe invasa
actcaagatcgtccggaactttatgaagaagtaaaattatacaagaatgccagagaacgagaaaagtatgaaaatcaagctgatttgtatgctgttgtcaacactttgcaacatttagagaaagcttacatcagagattgtgtcacacccaaagagtacactgcggcgtgcagcaagttgttagttcaatacagagcagctttcaaacaggttcaaagtgatcagtttccaacaattgatgcatttgccagagccttcaaacttgactgtccagcagctcttgagagaatcaaagaggacaggccaattacaattaaagatgacaagggcaatacatcgaagtgcattgctgacatagtttcactcttcattaccatcatggataaattacgcttggaaatcaaagctatggatcaacttcacccagatctcagagatttaatggataccatgaacagactcagtatactgccaagtgactttgatggaaaacagaaggttgcggagtggcttcagactttgaataatatgtctgcttcggacgaactgtcagatacgcaagttagacaattaatttttgatttagaaacgtcttacaatgcatttaacaaggttcttcacaattcttaa

SEQ ID NO: 30 - DNA, 100 bp, Leptocybe invasa
gatcaacttcacccagatctcagagatttaatggataccatgaacagactcagtatactgccaagtgactttgatggaaaacagaaggttgcggagtggc

SEQ ID NO: 31 - DNA, 408 bp, Leptocybe invasa
gaaaatgaaaatgtcgccgtcaagagaaaacgacagattgacacgctgaaaatctttaatgataatgtcttggaactgcaagaagatgttgaatatcaagtgctgttcaatgctggagaaagatgtatggcaattatggtctcattgtctccagactttccactggaaaaaccagtgctccgagtttcaccatcaataaagcataaatggtgtaatgaacacagtgaaattactagtgcaccaggattattaaattttacggttcatagtgaccttgggcgtgttgtgcaagctattattagggagttcagtaaaaatccaccacaactgttggaggaaaattctgcactcacagatgtagctggtagagtatcaccgtcgtacacatggaaaccacacgagcttgct

SEQ ID NO: 32 - DNA, 100 bp, Leptocybe invasa
cgtggagcgttttcttagtacgtacgtcgagtgtcggaagctcggccaggccaggcggaccaaggaggagaaactcacacatcaactcaacgagctaaag

SEQ ID NO: 33 - DNA, 336 bp, Leptocybe invasa
atgtacaagacatttcaagagcctgatattccagctgctataggtccagtttcaaaccttagcacgaacgaattaaaagatctactgaatgacgaggataagtttgaagaaatcatcaaaactaatcaacagttactggagtttgagtcggagaaagaagaaataatggtgaggaatcgatctttagcagaattcaatttatcaaaagaacctgaactagaagaagctaaaaatttcataaggtccctcagcgaagaaggcaatcaattgtgttccagtgttcaagccaaacttgaacaaataaaaaataatgctggtgcaatgtcaactgaaact

SEQ ID NO: 34 - DNA, 100 bp, Leptocybe invasa
tgacgaggataagtttgaagaaatcatcaaaactaatcaacagttactggagtttgagtcggagaaagaagaaataatggtgaggaatcgatctttagca

SEQ ID NO: 35 - DNA, 756 bp, Leptocybe invasa
atgaggagaaaaccaggagttggagcaatccaaaaacaaaaatatgaacaagaacggtatagagacaaaggaacggagcttcaagagaatcaattcgagcagatgactaagcaaatggaaacttttagaataaatttggaagagtttgcctcgaagcataagaatgaaataaagaaaaatgcacaattcaggcgtcagtttacagaaatgtgtgcttctataggtgtagatccattagcatctggcaaaggtttttggtctgtattaggtattggagatttctactatgaacttggaattcaaatagttgaagtatgtcttgcaactaattacaaaaatggaggcttgatatcgctggatgaactaagagaacgattaatccaagcaagaggacgtcgcaaagaacatcaagagataacaaacgaggatttacttgcttcagctaaaaaacttaaaatattaggcaatggattctctgtggtacccatcagtaaaggcaaatacttagttcagtctatacctggagagttgagcatggatcacactgcagtcttgcagcaaacaaataacaacggcaatgcctttatttcaaaatcattgctacaatcagaattaaaatgggagagtgaaagggctcaaaaagctcttgatcatatggtcaaagagggattagcttggattgataatcaaaatgaaggagaaccattgtactggtttccaagtttgttcactgcttgcattgcttcaaaaacgtaa

SEQ ID NO: 36 - DNA, 100 bp, Leptocybe invasa
cctggagagttgagcatggatcacactgcagtcttgcagcaaacaaataacaacggcaatgcctttatttcaaaatcattgctacaatcagaattaaaat

SEQ ID NO: 37 - DNA, 345 bp, Leptocybe invasa
atgactgaaattgattggccctggcaatacagttttcctccttttttcacacttcagccgcatgctgaaacaagggcaaaacaaattgacgcatggaagagtttaattctggattattttcgcacaacaaagcaagcaattcttgatgttcgtgaagttcacagcagcccattattcaacaatagttccattgatcgaaaactaccagtagaagttgtatcactgattcttgaggagctgtcaaagtcaggtaatgctaccccactagacaaatcaaagcaacgatgggttgtttcatggcatacacttgacgaatgggctgacatattgtactgttgggcacaa

SEQ ID NO: 38 - DNA, 100 bp, Leptocybe invasa
agttgtatcactgattcttgaggagctgtcaaagtcaggtaatgctaccccactagacaaatcaaagcaacgatgggttgtttcatggcatacacttgac

SEQ ID NO: 39 - DNA, 825 bp, Leptocybe invasa
atgaacaggttcgaatacgcggagcctcagttgttgccaaatgaaattcacgtcagacgcgatcgaggcatcagactctacgatggagacgtcaagagtcctttcgagggtggagaggtgacactgacaagccacagaataatttataaaacgcaagatggtctcacatttgccttaagacttagcttagttgtattttttgaggaggaaaatccaggagctttgttttttactcgcagcaagaaagtagtgttgcatttagctgagccacccagtgataaacttactggaccaattgatcatagccactataattatgttaagttgtcatttaaagagggactggatcctaattttgttgcacacctaagtgatacagttatcaaaaaactttggtagatagcgcctataaatgtttcgtcaatactcacaagctctccaaatgtgcaggggagttcaagtgcaaaaccactacctcaaattaaacccagaactggtattataggaatagaaagaagtatacaagagcaacagaaagccacagacgaaagcattaccgttgcctttcaggacttgaaaaagcttatggtcatggctaaggatatggttacaatatccaagaccatatctgcaaaaataagggaacgacaaggagacatcacagaggatgaaactgttcggttcaagtcatatttgatgagtttgggtatcgatgatcctgtgaccagagatgcctacagaactgagaatgaatattttaaacagcttgctcgacaggtagctgaagtactagaagagcctgtaaaggaagtgggtggaatgatg

SEQ ID NO: 40 - DNA, 100 bp, Leptocybe invasa
taaatgtttcgtcaatactcacaagctctccaaatgtgcaggggagttcaagtgcaaaaccactacctcaaattaaacccagaactggtattataggaat

SEQ ID NO: 41 - DNA, 687 bp, Leptocybe invasa
atggagtggctttttggaaaacgcgtaactcctgaggagatgctcaggaagaatcaaagagctttgaacaaggcaatgagagatctagacagagaaaggatgagaatggaacaacaggaaaagaaaatcatagctgatataaagaaaatggcaaaagatggacaaatggatgctgttaaaatcatggctaaagatcttgtcaggacaagacgctacgttaaaaagtttatgttaatgaaagcaaatattcaagcagtatctctgaaaattcagactttacgctctcaaaacacaatggctcaggccatgaaaggagtcacaaaggctatgcaaaatatgaacaaacaattaaatttgccacaaattcaaaagatattgcaagagtttgaaaaacaatctgagattatggatatgaaagaagaaataatgaatgatgcaatagatgatgccatggaagatgagggtgatgaagaagaaagcgatgcgattgtatcacaagttctggatgaacttggccttcaattaaacgaccagttgtcaggactacctcaagcctcaggatcgttgagcatagcaggttcgaagcaaccagtggccgcggcagctggagccggtaacgacgacggcaatctggcggatgccgatgcggatcttcatgcgcgacttgaaaatctgcgtcgcgagtag

SEQ ID NO: 42 - DNA, 100 bp, Leptocybe invasa
ctcaagcctcaggatcgttgagcatagcaggttcgaagcaaccagtggccgcggcagctggagccggtaacgacgacggcaatctggcggatgccgatgc

SEQ ID NO: 43 - DNA, 588 bp, Leptocybe invasa
ggttatcagattgacaggcaggttagagcaatccaaagagaagaagaaaaagtgaaaaaaacactgaaagaggcagctaaaaaaggtgacaaagatgtctgtaagattttggccaaagaagtgattagagcgcagaaagcttgtaagaagttgcatacctcgaaagcccacctgaattctgtcaccttgcaaatgaaaaatcaattagcaacaattagagttgctggctctgtctcaaaatcaacagaagtcatgcaagctatgcaagcgcttatcaaagttccagaggtcgctgcaacaatgcgagatttgtccaaagaaatgatgaaggctggtattattgaggaaatgatggatgaaactatggattctatggaggattctgaagaagtagaggaagctgctgatgaagaagttgacaagattctgtgggaagttactgctggacaaatgggaagagcaccagacgttgtcacagaaacacctggagcttcaacttcaaaagaagaagaagaaccagctgaagaactgagtgatgacaaggaattagaagaaatgaaaaatagattacaaagtcttcggagttag

SEQ ID NO: 44 - DNA, 100 bp, Leptocybe invasa
tgacaaagatgtctgtaagattttggccaaagaagtgattagagcgcagaaagcttgtaagaagttgcatacctcgaaagcccacctgaattctgtcacc

SEQ ID NO: 45 - DNA, 663 bp, Leptocybe invasa
atgagtttctttagcaaggtcttcggcgggaaaaaggagccgaccgccctaacgaccgccgaggcgatacaaaaactccgagagaccgaggagatgctcatcaagaaacaggacttccttgagaccaagatcacacaggaaatacaaacggccaggaagaacggtaccaagaacaaacgagctgccattcaagcgactgaaaaaaagaaacgatatgaaaagcagctacaacaaatcgatgggacgttgtccacaattgagatgcaaagagaagctctcgaaagtgcaaacacaaatactgctgttcttaccacaatgaagaatgcagctgatgctctcaaagctgcacatcaacacatgaatgtcgatgaagttcatgacatgatggatgatattgctgagcaacaagatgttgcaaaggaaatttcagatgcaatttcaaaccctgttgcatttggacaagatattgatgaagatgaactagagaaggagctagaagaattagaacaagaagagttagacaaagaattgctcggtatacaaaccacagatgaactacctgctgttcctgctacagccttaccagctgcacccgaaaagaaatcaaaaccaaaacaagaagaagatgatgatttaagagaactagagcaatgggcatcgtaa

SEQ ID NO: 46 - DNA, 100 bp, Leptocybe invasa
gccgaccgccctaacgaccgccgaggcgatacaaaaactccgagagaccgaggagatgctcatcaagaaacaggacttccttgagaccaagatcacacag

SEQ ID NO: 47 - DNA, 1800 bp, Ophelimus maskelli
atagcagaatgtgaagatgatgctactcacataatctatccatctgctgatcctctagaagaagaatacgcaaggccgtgttttcgaagagatcgatctgttttacttcactggtattattttccagatagttatgattcgtgggtcaacattgaacttccatgggattttcctgaaactgctcttggaaaccctcctccaaagtcaccatataaagtttctgctacttgggcactagatttagaacaatacaatgaatggatgaatgaggaagattatgaagtagatgaaagtggtcagaaaaaagtacataagtacaggttaagcgttgaagacatgatgacacaatcagcaccaccttctgttaaaaagcagaaaaggaagaggtctccgactccaccaccaaaactaggcaaacgaaaaagtgggcgagctcctgctggtcctcaaggtataaattcttctacgggtccaaagaaatcacgtggaactggagatgaagaagaagacttaactcaaggcatggatgatccacctgcagaacccaggataatggaagtcgtctctacaaatgctaacacaccaaactcagctcaaaacagtactggtcctatcatttccagtagcaaaaaacaggataatgaattgcagccattaaaatctggaaatatggccgatcttgatgaaccagtggatggtgaaaaaagtaactcacaaacgtctcaagaccgagaagaaagggatacgagtaaagaaaggaatgatggtagcaaaagcgatgaaccagaagataatgttacagaacaaactcatcacattgttgtacccagttactctgcgtggtttgattacaattccattcacacaatcgagaaacgagctctgtctgaatttttcaatggcaaaaacaaatcgaaaacaccagaaatttatttggcgtatagaaatttcatgatcgacacttatcgtttgaacccaacagagtacatcacgtcaacagcgtgcaggcgaaatttggctggtgatgtttgtgcgataatgcgcgtacatgctttcctcgaacaatggggtctaattaattatcaggtggatgccgattcaaggccgacgcctatgggtccaccgccaacctcacacttccacgtgctttcagacacaccgtcaggtttagcaccagttaatcctaatcctcctaaaacgccacagccatccgcagcgaaaactcttttggatctcgaaaagaagcctgttattacggatgaaaaagttcctccggtcggacccatggcaaactttggtctcaagatcgatcagtattcgaaaaaaccagccgtactgaaaaataaacaagctgctggtgcaactcgtgattggacggaacaagaaacgttattattactagaggctttagaacttcataaggacgattggaataaggtgtgcgagcacgttggctcgaggacacaagatgagtgcattctgcatttcttaaggctacccattgaggatccataccttgaggagccggagggtctaggcccgttggcatatcagcctattcctttctctaaggctggaaatcctgttatgagtacagtagctttcctggcatcagtggttgatccaagagtagcggcaagtgcggccaaagctgcaatggaagaattcgctgcgataaaggatcaggtgccagcagcacttctggatcaacatttaaggaatgtgcaggctagcgcgaattcggatggtaaattcgatccggcagctggtcttgcacagtcgggtatcgccggaaccggt

SEQ ID NO: 48 - DNA, 100 bp, Ophelimus maskelli
atcgacacttatcgtttgaacccaacagagtacatcacgtcaacagcgtgcaggcgaaatttggctggtgatgtttgtgcgataatgcgcgtacatgctt

SEQ ID NO: 49 - DNA, 1530 bp, Leptocybe invasa
cagtacaatgagtggatgaatgaagaagattatgaagtagatgatagtggacagaaaaaggtacataaatatagactatcagttgaggacttgatggctccaacacctgcatcaggtaaaaaacaaaagagaaaaagtcgcccagtcctcctccaaaacttggaaaaagaaaagtggcagagctcctggaggaactcaaggtgggttgtccggcccaaaaaatcacgaggtggagacgaagaagaagacttgacacaaggaatggaggatcctccgtctgagccgagaataacggaagttgtaaattcgaatacgaatgcatctatctctggacagaatagcagctcaggcatggtgtccagcaaaaaacaggacaatgacatgcagccactcaagtctggaaacatggccgatttggatgaaccagttgatggtgataaaagcaattcgcaaaattcacaagacagagaagaacgtgacacgagcaaggaaagaggcgacggcagcaagagcgatgagcccgaggataacgtgaccgagcagactcatcacattgtgattccgagctactcggcgtggtttgactacaactctattcacatgattgagaagcgagcactatcagagtttttcaacggcaagaacaagtctaagacaccagaaatctacctcgcttacaggaatttcatgatcgacacctatcgcctcaatccgaccgagtacattacttccacagcctgtaggcgaaacttagctggtgatgtatgcgctatcatgcgcgtgcacgcttttctcgaacagtggggtctgatcaattatcaagtggatgccgattcaagaccgacacctatgggacctccacctacttcgcacttccatgttttgtcagatacaccgtctgggttagctccagttaatccaaaccctcccaaaacaccgcagccgtcagcggcaaagacgttacttgatctcgaaaagaaaccgattattgacgagaagattccagctgctggagcgatggccaacttcggcctgaagctcgatcaatacgcgaggaagcctgcggttttgaaaaacaagcaagctgctggtgctactcgcgagtggacggaacaagaaacgctgttattgcttgaagctttggagttacacaaggatgactggaataaggtttgtgagcatgttggttcaagaactcaggatgaatgtatcctacactttttgcgattacccattgaagacccgtaccttgaagaaccagaaggtcttggtccactggcatatcagcccatacctttttctaaggctggtaaccctgtcatgagcaccgttgcatttttggcttcggtagtggatcctagagtagcagctagtgcggcaaaggctgctatggaagagtttgctgccatcaaagaccaggtgcctgctgctttactggatcaacatttgagaaatgttcaggctactgctgctgatggaaaattcgacccagccgccggtcttgcacagtcaggtatcgcaggcacagga

SEQ ID NO: 50 - DNA, 100 bp, Leptocybe invasa
agaggcgacggcagcaagagcgatgagcccgaggataacgtgaccgagcagactcatcacattgtgattccgagctactcggcgtggtttgactacaact

SEQ ID NO: 51 - DNA, 981 bp, Ophelimus maskelli
atgaaaccactgatgttacatgggcacgagcgtgccatcacaaaaatcaaatacaacagagaaggagacttacttttttctgctagtaaagataaacaaccgaatgtttggtattcgttaaacggagaaagacttggaacattcaatggacacaatggttcagtttggtgtattgatgtcaattgggatacaacacgctttttatccggtagtggtgacaacactttaagattatgggattgtgcaacaggaaaagagattagtcaattgtccacaaacagttcagtaagagcttgtgcatttagctattcaggcaatcttgctgtatatgccactgacaaagctcttggacaccaatgtgaaatgtttattattgacattaggtctcctgaaagtgttctttctcaagatgataacgtttgtagaacttcagtcagtggttcaaggatttcatctctcttgtggggagctctcgatgaatctattattactggtcatgaaaatggtgatttaaacatctgggacagtaggactggaaagaaattgagtgatgctcagggtcacaagggtcaaattaatgacatgcagttcaacaaggatggaactatgttcgttacagcctcaaaggaccacactgcaaagttgtttgacagtgaatctcttgttccattgaaaacgtataaaacagaaaggccagttaactctgccacgatatctccaatctttgatcacgtcgtagttggaggtggtcaagacgctatggacgtcaccacgacatcgacgaaacaaggaaaattcgatgctcgttttttccatcttgtctttgaagaagaatttgcacgtttaaaaggtcactttggtccaattaactcgctggcctttcatccaaatggacgcagtctgtctactggaggagaggatggttatattcgtataaatacatttgatcagtcctatttcgattttcattttgagtactaa

SEQ ID NO: 52 - DNA, 100 bp, Ophelimus maskelli
tgacattaggtctcctgaaagtgttctttctcaagatgataacgtttgtagaacttcagtcagtggttcaaggatttcatctctcttgtggggagctgtc

SEQ ID NO: 53 - DNA, 939 bp, Leptocybe invasa
atgaaacctttaatgttgcacgggcacgagcgtgccattacaaaaattaagtataacagagaaggagatttactgttttcagccagtaaagacaaacagcctaatgtatggtactctctcaatggtgaaagattgggtacattcaatggccacaacggttcagtttggtgcattgacgtcaattgggatactacacgcttcctttctggcagtggtgacaacacactgagaatatgggattgtcaaacaggcaaagaaataagccagttgtcaaccaacagttctgtaagagcttgcgctttcagttactctggtaaccttgctgtttatgcaactgacaaagctcttggtcatcagtgtgaaatgttcattattgatattaggactcctgaaagtgtactttctcaggaagataatgtctgcagaactatgatcggtggttccaggatctcatctctcctttggggagctcttgatgaaaccatcataactggtcatgagaatggtgacttgaccatttgggatagtaggactggaaagaaattgagcgacgctcagggacacaaaggtcaaataaatgatatgcagttcaacaaagatgccactatgttttgcactgcctccaaagatcacactgctaaattatttgacagcgaatctctagtttcactaaaaacttacaaaactgagcggcccgttaactcggctacgatttctccaatctttgatcatgtcgttgttggaggtggtcaagatgccatggatgtaacaacgacgtcgacgaaacaaggaaaattcgacgctcgtttttatcatctcgtctttgaagaagaatttgcacgtttaaagggccatttcggccccatcaactcattatccttccacccgaacggcagaagcttagccactggaggagaggacggttacattcgtatcaac

SEQ ID NO: 54 - DNA, 100 bp, Leptocybe invasa
acgtttaaagggccatttcggccccatcaactcattatccttccacccgaacggcagaagcttagccactggaggagaggacggttacattcgtatcaac

SEQ ID NO: 55 - DNA, 706 bp, Artificial Sequence
  source: /note=“Description of Artificial Sequence Synthetic polynucleotide”
atgagcttcttcacgaaggtcttcggcgggaagaaggaggcggctgctccgacgacctcggaggccatacagaaactacgcgagacggaggagatgctcaatcgacacttatcgtttgaacccaacagagtacatcacgtcaacagcgtgcaggcgaaatttggctggtgatgtttgtgcgataatgcgcgtacatgctttgacattaggtctcctgaaagtgttctttctcaagatgataacgtttgtagaacttcagtcagtggttcaaggatttcatctctcttgtggggagctgtcggctcgaacgagccgactaattgtctttaaacgcgcgatataagcgcacaatgctcgagaaacgataaactctatcgctctgtcgcgtgcgtggcatcttcgcgcggacagctccccacaagagagatgaaatccttgaaccactgactgaagttctacaaacgttatcatcttgagaaagaacactttcaggagacctaatgtcaaagcatgtacgcgcattatcgcacaaacatcaccagccaaatttcgcctgcacgctgttgacgtgatgtactctgttgggttcaaacgataagtgtcgattgagcatctcctccgtctcgcgtagtttctgtatggcctccgaggtcgtcggagcagccgcctccttcttcccgccgaagaccttcgtgaagaagctcat

SEQ ID NO: 56 - DNA, 706 bp, Artificial Sequence
  source: /note=“Description of Artificial Sequence Synthetic polynucleotide”
gccgaccgccctaacgaccgccgaggcgatacaaaaactccgagagaccgaggagatgctcatcaagaaacaggacttccttgagaccaagatcacacagagaggcgacggcagcaagagcgatgagcccgaggataacgtgaccgagcagactcatcacattgtgattccgagctactcggcgtggtttgactacaactacgtttaaagggccatttcggccccatcaactcattatccttccacccgaacggcagaagcttagccactggaggagaggacggttacattcgtatcaacggctcgaacgagccgactaattgtctttaaacgcgcgatataagcgcacaatgctcgagaaacgataaactctatcgctctgtcgcgtgcgtggcatcttcgcgcggttgatacgaatgtaaccgtcctctcctccagtggctaagcttctgccgttcgggtggaaggataatgagttgatggggccgaaatggccctttaaacgtagttgtagtcaaaccacgccgagtagctcggaatcacaatgtgatgagtctgctcggtcacgttatcctcgggctcatcgctcttgctgccgtcgcctctctgtgtgatcttggtctcaaggaagtcctgtttcttgatgagcatctcctcggtctctcggagtttttgtatcgcctcggcggtcgttagggcggtcggc

SEQ ID NO: 57 - DNA, 914 bp, Artificial Sequence
  source: /note=“Description of Artificial Sequence Synthetic polynucleotide”
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagatatttttacaacaattaccaacaacaacaaacaacaaacaacattacaattactatttacaattaca

SEQ ID NO: 58 - DNA, 301 bp, Artificial Sequence
  source: /note=“Description of Artificial Sequence Synthetic polynucleotide”
tttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 59 - DNA, 921 bp, Artificial Sequence
  source: /note=“Description of Artificial Sequence Synthetic polynucleotide”
gtgtgtcttgtcttatctggttcgtggtggtgagtttgttacaaaaaaatctattttccctagttgagatgggaattgaactatctgttgttatgtggattttattttcttttttctctttagaaccttatggttgtgtcaagaagtcttgtgtactttagttttatatctctgttttatctcttctattttctttaggatgcttgtgatgatgctgtttttttttgtccctaagcaaaaaaatatcatattatatttggtccttggttcatttttttggtttttttttgtcttcacatataaatattgtttgaatgtcttcaatcttttatttgtatgagacaattatttaagtatcgggtgacaatgcagctattatgtattgtcgattgttatattggcgcccaaaatatatacttagcctaagaatttggtaagtgagtggcttatgttttactccagcaaaaattgtgtgtgtattaccattctgatgcgaaacaagaaaagaatttgatctaagaaaccaagtttattcactagttaaaaaacaaatgacctaatgtaatcgactccacatatcaaaatacgtaaaacaaacattgtatgttgacaaaagggaaaagaaatgatttatttggttaaaaagaaagctggattcaattgcaacagtttagtcgaaatcattttgaaaggcttacaatggattgaatgtgaatattccattaagccgcttctgtctacacagaatgttacgcttggagagcagcaatcattttcacgtttttatctttttaggtggacatgtatattattggttacgcctttggagtttttcgaaatttatttctttcaaatcacaagatgactaaacatcacaatctgtttatcttcctaactagttaaatttttgtccccaccatt

SEQ ID NO: 60 - DNA, 253 bp, Artificial Sequence
  source: /note=“Description of Artificial Sequence Synthetic polynucleotide”
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 61 - DNA, 106 bp, Artificial Sequence
  source: /note=“Description of Artificial Sequence Synthetic polynucleotide”
ggctcgaacgagccgactaattgtctttaaacgcgcgatataagcgcacaatgctcgagaaacgataaactctatcgctctgtcgcgtgcgtggcatcttcgcgcg

SEQ ID NO: 62 - DNA, 300 bp, Artificial Sequence
  source: /note=“Description of Artificial Sequence Synthetic polynucleotide”
atgagcttcttcacgaaggtcttcggcgggaagaaggaggcggctgctccgacgacctcggaggccatacagaaactacgcgagacggaggagatgctcaatcgacacttatcgtttgaacccaacagagtacatcacgtcaacagcgtgcaggcgaaatttggctggtgatgtttgtgcgataatgcgcgtacatgctttgacattaggtctcctgaaagtgttctttctcaagatgataacgtttgtagaacttcagtcagtggttcaaggatttcatctctcttgtggggagctgtc

SEQ ID NO: 63 - DNA, 300 bp, Artificial Sequence
  source: /note=“Description of Artificial Sequence Synthetic polynucleotide”
gccgaccgccctaacgaccgccgaggcgatacaaaaactccgagagaccgaggagatgctcatcaagaaacaggacttccttgagaccaagatcacacagagaggcgacggcagcaagagcgatgagcccgaggataacgtgaccgagcagactcatcacattgtgattccgagctactcggcgtggtttgactacaactacgtttaaagggccatttcggccccatcaactcattatccttccacccgaacggcagaagcttagccactggaggagaggacggttacattcgtatcaac

SEQ ID NO: 64 - DNA, 306 bp, Artificial Sequence
  source: /note=“Description of Artificial Sequence Synthetic polynucleotide”
atgagcttcttcacgaaggtcttcggcgggaagaaggaggcggctgctccgacgacctcggaggccatacagaaactacgcgagacggaggagatgctcaggctcgaacgagccgactaattgtctttaaacgcgcgatataagcgcacaatgctcgagaaacgataaactctatcgctctgtcgcgtgcgtggcatcttcgcgcgtgagcatctcctccgtctcgcgtagtttctgtatggcctccgaggtcgtcggagcagccgcctccttcttcccgccgaagaccttcgtgaagaagctcat

SEQ ID NO: 65 - DNA, 506 bp, Artificial Sequence
  source: /note=“Description of Artificial Sequence Synthetic polynucleotide”
atgagcttcttcacgaaggtcttcggcgggaagaaggaggcggctgctccgacgacctcggaggccatacagaaactacgcgagacggaggagatgctcaaatgtattgctgacattgtttcactcttcataacattaatggataagttacgacttgaaattaaagctatggatcaacttcatccagatcttagagatttggctcgaacgagccgactaattgtctttaaacgcgcgatataagcgcacaatgctcgagaaacgataaactctatcgctctgtcgcgtgcgtggcatcttcgcgcgaaatctctaagatctggatgaagttgatccatagctttaatttcaagtcgtaacttatccattaatgttatgaagagtgaaacaatgtcagcaatacatttgagcatctcctccgtctcgcgtagtttctgtatggcctccgaggtcgtcggagcagccgcctccttcttcccgccgaagaccttcgtgaagaagctcat

SEQ ID NO: 66 - DNA, 200 bp, Artificial Sequence
  source: /note=“Description of Artificial Sequence Synthetic polynucleotide”
atgagcttcttcacgaaggtcttcggcgggaagaaggaggcggctgctccgacgacctcggaggccatacagaaactacgcgagacggaggagatgctcaaatgtattgctgacattgtttcactcttcataacattaatggataagttacgacttgaaattaaagctatggatcaacttcatccagatcttagagattt

SEQ ID NO: 67 - DNA, 306 bp, Artificial Sequence
  source: /note=“Description of Artificial Sequence Synthetic polynucleotide”
gccgaccgccctaacgaccgccgaggcgatacaaaaactccgagagaccgaggagatgctcatcaagaaacaggacttccttgagaccaagatcacacagggctcgaacgagccgactaattgtctttaaacgcgcgatataagcgcacaatgctcgagaaacgataaactctatcgctctgtcgcgtgcgtggcatcttcgcgcgctgtgtgatcttggtctcaaggaagtcctgtttcttgatgagcatctcctcggtctctcggagtttttgtatcgcctcggcggtcgttagggcggtcggc

SEQ ID NO: 68 - DNA, 506 bp, Artificial Sequence
  source: /note=“Description of Artificial Sequence Synthetic polynucleotide”
gccgaccgccctaacgaccgccgaggcgatacaaaaactccgagagaccgaggagatgctcatcaagaaacaggacttccttgagaccaagatcacacaggatcaacttcacccagatctcagagatttaatggataccatgaacagactcagtatactgccaagtgactttgatggaaaacagaaggttgcggagtggcggctcgaacgagccgactaattgtctttaaacgcgcgatataagcgcacaatgctcgagaaacgataaactctatcgctctgtcgcgtgcgtggcatcttcgcgcggccactccgcaaccttctgttttccatcaaagtcacttggcagtatactgagtctgttcatggtatccattaaatctctgagatctgggtgaagttgatcctgtgtgatcttggtctcaaggaagtcctgtttcttgatgagcatctcctcggtctctcggagtttttgtatcgcctcggcggtcgttagggcggtcggc

SEQ ID NO: 69 - DNA, 200 bp, Artificial Sequence
  source: /note=“Description of Artificial Sequence Synthetic polynucleotide”
gccgaccgccctaacgaccgccgaggcgatacaaaaactccgagagaccgaggagatgctcatcaagaaacaggacttccttgagaccaagatcacacaggatcaacttcacccagatctcagagatttaatggataccatgaacagactcagtatactgccaagtgactttgatggaaaacagaaggttgcggagtggc

SEQ ID NO: 70 - DNA, 729 bp, Ophelimus maskelli
atcgatgagagagaacctcctccagaaggatttagtatgatctctcacactgtagatacaatgcaaaaagcgtggcgtaaaagacaactgtgttataaaattagaactagagacttatgcccatgcgcagtatcggatataattatttgcaataggattggtaaaggctcaaaaatggcaccaaatgaatttacttatgctggtgtcatcaatggagtttatatttgttacaaaactgtacccctctctgcaagcccgttatactctgcacagtcatatgctaatatagaattattccaaacgttgtctccaactctgtcaaacggtacaccgcacagagtggcaccagagaggccaccaaagccaaaattcacgccgaaacctacaaatggactgtatccgcaaataagtccagtaaacgcaggaaagactgcagcggatgagaatgaccaagactatgaagtactaagtccaagtgccagaataaggccaacgcgaccagcgccaaggccaccgtctaataaatcgccattggttcattctgtgccagcttatggaacattgacaggttcatcagatctcgacggagttccttttgttctcaatcctcttttgagcatgagtacatcaattatgttaaataaacagtttcctgtgattaaaatccgaactcagaacgagctggataaagagtatttctacgactttcgaactgagagagaaacttga

SEQ ID NO: 71 - DNA, 117 bp, Ophelimus maskelli
caaaaagctttagatcatatggttaaagaaggattagcttggatagatgaacaaaatgaagaacccttgtactggtttccaagtctttttacagcttgcattgcttcaaaaacatag

SEQ ID NO: 72 - DNA, 153 bp, Leptocybe invasa
agtcaagagcaaagcgaagtgatagcccaggactttcttgatcgcaaaattgacgtggagcgttttcttagtacgtacgtcgagtgtcggaagctcggccaggccaggcggaccaaggaggagaaactcacacatcaactcaacgagctaaag

SEQ ID NO: 73 - DNA, 153 bp, Leptocybe invasa
ttttatgaactaacccaaggagaggatactgctgatcaagaatttcatggcttagataacgaagtacttgtgagagctttaagaacattagaagcatctaaaaaggctgaattaataatgtttgatgataatgaaggtgtaaagtttttctaa

SEQ ID NO: 74 - DNA, 225 bp, Leptocybe invasa
caggcaagagttcacagtgattatgaagttgccgaagctgtgtcacaattgataaaagacagaggatcaattacagctacggaattagcacaatcagcaggaatttctgtggttttggcctgtgaaaggctactgatgacagagaaatacggaaaagcctgcagagatgattcgatcgaagcattgagattttaccctaatttattcctggaacaggagtcgtga

(19) United States
(12) Patent Application Publication
(10) Pub. No.: US 2015/0259701 A1
(43) Pub. Date: Sep. 17, 2015

(54) GALL WASP CONTROL AGENTS

(71) Applicant: FUTURAGENE ISRAEL LTD., Rehovot (IL)

(72) Inventor: Dror Avisar, Kocha Yair (IL)

(73) Assignee: Futuragene Israel Ltd., Rehovot (IL)

(21) Appl. No.: 14/432,431

(22) PCT Filed: Oct. 2, 2013
(86) PCT No.: PCT/IB13/02484

§ 371 Date: Mar. 30, 2015

Related U.S. Application Data
(60) Provisional application No. 61/744,771, filed on Oct. 3, 2012.

(51) Int. Cl. C12N 15/82; A01G 1/00; C12N 15/113

(52) U.S. Cl. CPC C12N 15/8286; C12N 15/1137; C12N 15/113; A01G 1/001; C12N 2310/14; C12N 2310/531

(57) ABSTRACT

The present invention relates to the field of doublestranded RNA (dsRNA)-mediated gene silencing in insect species. The present invention is based, in part, on the inventors' sequencing of genes from eucalyptus invasive species gall wasp pests Leptocybe invasa (Li) and Ophelimus maskelli (Om). In certain aspects, the invention provides Li and Om nucleic acids, derivatives thereof and the use of such nucleic acids and derivatives as gall wasp control agents.

### SEQUENCE LISTING

The instant application contains a Sequence Listing which has been submitted in ASCII format via EFS-Web and is hereby incorporated by reference in its entirety. Said ASCII copy, created on Sep. 27, 2013, is named 30407-0006WO1_SL.txt and is 46, 878 bytes in size.

### FIELD OF THE INVENTION

The present invention relates to the field of double stranded RNA (dsRNA)-mediated gene silencing in insect species.

### BACKGROUND

Gall wasp infestations of eucalyptus trees have occurred in both the Northern and Southern hemispheres and pose a threat to commercial eucalyptus farming in China, Australia, Israel and Brazil. Efforts to control gall wasp infection of eucalyptus have included attempts to isolate naturally resistant plants and natural predators. These efforts have met with limited or no success. The protective environment of the gall in which gall wasps develop makes chemical pesticide control of gall wasps difficult.

Even when feasible, chemical pesticide control has disadvantages. Chemical pesticides are potentially detrimental to the environment, are not selective and are potentially harmful to non-target crops and fauna. Chemical pesticides persist in the environment and generally are metabolized slowly, or not at all. Chemical pesticides accumulate in the food chain, particularly in the higher predator species where they can act as mutagens and/or carcinogens to cause irreversible and deleterious genetic modifications. Crop pests, moreover, may develop resistance against chemical insecticides because of repetitive usage of the same insecticide or of insecticides having the same mode of action.

RNA interference or “RNAi” is a process of sequence-specific down-regulation of gene expression (also referred to as “gene silencing” or “RNA-mediated gene silencing”) initiated by double-stranded RNA (dsRNA) that is complementary in sequence to a region of the target gene to be down-regulated. Down-regulation of target genes in multicellular organisms by means of RNA interference (RNAi) has become a well-established technique. U.S. patent application publications US 2009/0285784 A1 and US 2009/0298787 relate to dsRNA as an insect control agent and are hereby incorporated herein by reference in their respective entireties. U.S. Pat. No. 6,506,559, U.S. patent application publication 2003/00150017 A1, International Publications WO 00/01846, WO 01/37654, WO 2005/019408, WO 2005/049841, WO 05/047300 relate to the use of RNAi to protect plants against insects. Each of the foregoing patents and published applications is hereby incorporated by reference in its entirety.

Additional gall wasp control agents and methods of use thereof are disclosed in International application no. PCT/US2012/031413, filed Mar. 30, 2012, which is hereby incorporated by reference in its entirety.

### SUMMARY

The present invention is based, in part, on the inventors' sequencing of genes from eucalyptus invasive species gall wasp pests, Leptocybe invasa (Li) and Ophelimus maskelli (Om). In certain aspects, the invention thus provides Li and Om nucleic acids, derivatives thereof and the use of such nucleic acids and derivatives as gall wasp control agents.

In certain aspects the invention provides isolated nucleic acids that hybridize selectively under high stringency hybridization conditions to a sequence set out in SEQ ID NO: 1-54 and 70-74 and complementary sequences thereof.

In certain aspects the invention provides isolated nucleic acids that are 90-99.99 percent identical to sequences set out in SEQ ID NO: 1-54 and 70-74 and complementary sequences thereof.

In certain aspects the invention provides isolated nucleic acids that include at least 17 contiguous nucleotides of the sequences set out in SEQ ID NO: 1-54 and 70-74.

In certain aspects the invention provides nucleic acids from Li or Om, including the nucleic acids set out above, that are about 80% or less identical to the honey bee ortholog of said nucleic acid.

In certain aspects the invention provides vectors that include nucleic acids from Li or Om, or reverse compliments of such sequences, operably linked to an expression control sequence.

In certain aspects the invention provides host cells transformed with and/or harboring vectors that include nucleic acids from Li or Om, or reverse compliments of such sequences, operably linked to an expression control sequence.

In certain aspects the invention provides plant tissues, for example, leaf tissue and seeds, transformed with and/or harboring vectors that include nucleic acids from Li or Om operably linked to an expression control sequence.

In certain aspects the invention provides isolated small inhibitory ribonucleic acid (siRNA) molecules that inhibit expression of Li or Om nucleic acids.

In certain aspects the invention provides isolated double stranded ribonucleic acid (dsRNA) molecules that include a first strand of nucleotides that is substantially identical to at least 17 contiguous nucleotides of SEQ ID NO: 1-54 and 70-74, and a second strand of nucleotides that is substantially complementary to the first strand of nucleotides.

In certain aspects the invention provides double stranded ribonucleic acid (dsRNA) molecules with a high level of homology (greater than 80%) to mRNA from Li or Om (Li or Om targeting dsRNAs), including the dsRNA molecules set out above, that are about 80% or less identical to the honey bee ortholog of the dsRNA.

In certain aspects the invention provides vectors that include an expression control sequence operatively linked to a nucleotide sequence that is a template for one or both strands of a dsRNA from Li or Om.

In certain aspects the invention provides host cells transformed with and/or harboring vectors that include an expression control sequence operatively linked to a nucleotide sequence that is a template for one or both strands of a dsRNA from Li or Om.

In certain aspects the invention provides plant tissue transformed with and/or harboring vectors that include an expression control sequence operatively linked to a nucleotide sequence that is a template for one or both strands of a dsRNA from Li or Om.

In certain aspects the invention provides isolated small inhibitory ribonucleic acid (siRNA) molecules that inhibit expression of an essential gene of Li or Om.

In certain aspects the invention provides methods of producing a pest resistant plant by expressing a Li or Om dsRNA in the plant or in propagative or reproductive material of the plant.

In certain aspects the invention provides methods of producing pest resistant eucalyptus by expressing a Li or Om dsRNA in the eucalyptus or in propagative or reproductive material of the eucalyptus.

In certain aspects the invention provides methods of producing eucalyptus resistant to gall wasp infection and/or infestation by expressing a Li or Om targeting dsRNA in the eucalyptus or in propagative or reproductive material of the eucalyptus.

In certain aspects the invention provides methods of producing a plant resistant to a plant pathogenic pest by transforming a plant cell with a recombinant DNA construct or combination of constructs that express a dsRNA; regenerating a plant from the transformed plant cell; and growing the transformed plant cell under conditions suitable for the expression of the recombinant DNA construct.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 schematically depicts certain, non-limiting nucleic acids according to the invention. (A) Schematic of silencing construct constructed using sequences from three gall wasp genes. Transgene P1 (Promoter 1) to T1 (Termination sequence 1) encodes a hairpin RNA (hpRNA) for silencing gall wasp, constructed by fusing 100 bp from each of three different gall wasp genes (Gw1, Gw2 and Gw3), by synthesizing the resulting sequence as an inverted repeat, and inserting a loop sequence between the respective sense and inverted repeat sequences. Transgene P2 (Promoter 2) to T2 (termination sequence 2) encodes an mRNA with the respective fused 100 bp sequences from the three gall wasp genes. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

FIG. 2 schematically depicts certain, non-limiting nucleic acids according to the invention. (A) Schematic of Om silencing construct #1 (SEQ ID NO: 55), constructed from sequences from three Om genes in accordance with the general scheme depicted in FIG. 2 (B) Schematic of hpRNA molecule produced by transcription of transgene P1 to T1. (C) Schematic of mRNA produced by transcription of transgene P2 to T2. Definitions: P1—CaMV 35S Promoter (SEQ ID NO: 57); P2—sgFIMV Promoter (SEQ ID NO: 58); T1—AtActin7 Terminator (SEQ ID NO: 59); T2—NOS Terminator (SEQ ID NO: 60); L—loop sequence site (SEQ ID NO: 61); Om 1—SEQ ID NO: 24; Om 2—SEQ ID NO: 48; Om 3—SEQ ID NO: 52.

FIG. 3 schematically depicts certain, non-limiting nucleic acids according to the invention. (A) Schematic of Li silencing construct #2 (SEQ ID NO: 56), constructed from sequences from three Li genes in accordance with the general scheme depicted in FIG. 1 (B) Schematic of hpRNA molecule produced by transcription of transgene P1 to T1. (C) Schematic of mRNA produced by transcription of transgene P2 to T2. Definitions: P1—CaMV 35S Promoter (SEQ ID NO: 57); P2—sgFIMV Promoter (SEQ ID NO: 58); T1—AtActin7 Terminator (SEQ ID NO: 59); T2—Nos Terminator (SEQ ID NO: 60); L—loop sequence site (SEQ ID NO: 61); Li 1—SEQ ID NO: 46; Li 2—SEQ ID NO: 50; Li 3—SEQ ID NO: 54.

FIG. 4 schematically depicts certain, non-limiting nucleic acids according to the invention. (A) Schematic of silencing construct constructed using sequences from a single Gw gene. Transgene P1 to T1 encodes a hairpin RNA (hpRNA) for silencing Gw, constructed from 100 bp of a Gw gene, by synthesizing the sequence as an inverted repeat, and inserting a loop sequence between the respective sense and inverted repeat sequences. Transgene P2 to T2 encodes an mRNA with the 100 bp sequence from the Gw gene. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

FIG. 5 schematically depicts certain, non-limiting nucleic acids according to the invention. (A) Schematic of silencing construct constructed using sequences from two Gw genes. Transgene P1 to T1 encodes a hairpin RNA (hpRNA) for silencing Gw, constructed by fusing 100 bp from each of two different Gw genes, by, synthesizing the resulting sequence as an inverted repeat, and inserting a loop sequence between the respective sense and inverted repeat sequences. Transgene P2 to T2 encodes an mRNA with the respective fused 100 bp sequences from the two Gw genes. mRNA transcribed from transgene P2 to T2 is the template for cytoplasmic enhancement of the silencing signal. (B) Schematic of hpRNA molecule produced by transcription of transgene P1 to T1. (C) Schematic of mRNA produced by transcription of transgene P2 to T2.

Like reference symbols in the various drawings indicate like elements.

### DETAILED DESCRIPTION

The present invention relates to using double stranded RNA (dsRNA)-mediated techniques to control insect infection and infestation of plants. The inventors have conducted transcriptome sequencing of the natural eucalyptus pests, Leptocybe invasa (Li) and Ophelimus maskelli (Om) and mined the respective transcriptomes to identify open reading frames of Li and Om genes that correspond to Li and Om mRNAs. The identification of Li and Om RNAs allows for the design of siRNA and dsRNA that mediate downregulation (silencing) of Li and Om genes. Such siRNA and dsRNAs are thus useful as biological control agents to kill or inhibit the development of Li and Om and inhibit infection of plants by Li and Om.

Accordingly, the present invention describes a nucleic acid based approach for the control of gall wasp pests. The active ingredient is a nucleic acid, for example a double-stranded RNA (dsRNA) or a nucleic acid that can promote or lead to production of a dsRNA, which can be used as an insecticidal formulation. dsRNA can be expressed in a host plant, plant part, plant cell or seed to protect the plant against gall wasps. The sequence of the dsRNA corresponds to part or whole of an essential gall wasp gene and causes downregulation of the insect target gene via RNA interference (RNAi). As a result of the downregulation of mRNA, the dsRNA prevents expression of the target insect protein and causes death, growth arrest or sterility of the insect.

The methods of the invention find practical application in any area of technology where it is desirable to inhibit viability, growth, development or reproduction of gall wasps, or to decrease pathogenicity or infectivity of the insect. The methods of the invention further find practical application where it is desirable to specifically down-regulate expression of one or more target genes in a gall wasp insect. Particularly useful practical applications include, but are not limited to, protecting plants against gall wasp pest infestation.

siRNA control of insect growth, for preventing insect infestation of a cell or a plant susceptible to insect infection, is effected by contacting insects with a dsRNA produced by annealed complementary strands, one of which has a nucleotide sequence which is complementary to at least part of the nucleotide sequence of an insect target gene. dsRNA is expressed in plant tissue that is ingested by the insect and then taken up by the insect through the gut, and thereby controls growth or prevents infestation. See Huvenne et al., 2010, J Insect Physiol 56: 227-35.

Gall wasp target genes for siRNA-mediated intervention include are preferably non-redundant, vital genes. Vital target genes may be any gene that when inhibited interferes with growth or survival or pathogenicity or infectivity of the insect. Such vital target genes are essential for viability, growth, development or reproduction of the insect, or any gene that is involved with pathogenicity or infectivity of the insect, such that specific inhibition of the target gene leads to a lethal phenotype or decreases or stops insect infestation. Down regulation of such vital target genes, whose activity cannot be complemented by other related genes, results in significant damage to the pest larvae and provides an efficient pest control system for sessile gall wasp pests. The target gene may be any of the target genes herein described, for instance a target gene that is essential for the viability, growth, development or reproduction of the pest. Examples of target genes include, for example, genes that are involved in protein synthesis and/or metabolism and/or RNA synthesis and metabolism and/or cellular processes. A slight knockdown of these target genes will have an effect on many other genes and processes ultimately leading to a lethal effect on the target pest. Such a down-regulated target gene will result in the death of the insect, or the reproduction or growth of the insect being stopped or delayed. Such target genes are vital for the viability of the insect and are referred to as vital genes.

Proper sequestration of transmembrane signaling receptors is crucial for cell function. Endosomal cargo sorting utilizes the biogenesis of multivesicular bodies to compartmentalize transmembrane receptors and hence, regulate signal transduction. This is due to the presence of active signal transduction via the cytoplasmic domain of the receptor even after internalization into intraluminal vesicles intracellularly. Any signal transduction is subsequently halted upon removal of the signaling domain in the intraluminal vesicle during multivesicular body biogenesis. Once the receptor is in the multivesicular body, the vesicle fuses to a lysosome where it is fully degraded. (17, 18, 19) The initial sorting of cargo into vesicles is regulated by monoubiquitylation, by the Hrs-STAM complex and the endosomal sorting complexes responsible for transport: ESCRT (Endosomal Sorting Complex Required for Transport) ESCRT-I, -II, -III. Mutations in these components result in the improper deliver of cargo to the lysosome. Evidence of the role of the ESCRT protein family in endosomal sorting and sequestration has been shown in yeast, Drosophila and in humans. In yeast, mutations in these specific sorting complexes leads to exclusion of cargo from the yeast lysosome, the vacuole, resulting in a phenotype in which there is an absence of intraluminal vesicle-containing multivesicular bodies. Metazoan ESCRT activity has an impact on developmental processes in Drosophila. (17, 18) Genes in the western corn rootworm Diabrotica virgifera virgifera LeConte have been identified whose RNA interference resulted in statistically significant larval stunting and mortality (17-20). Orthologs of these ESCRT protein family members in Drosophila include Dmel, Vps23, Vps28, Vps37/mod(r), and Vps37b which comprise the ESCRT I complex; Vps22/Isn, Vps25, and Vps36 which make up ESCRT II; and Vps2, Vps20, Vps24, and Snf7/shrub which comprise the ESCRT complex. The presence of dsRNA of certain protein orthologs in corn rootworm have been shown to decrease levels of their respective RNA. Drosophila harboring ESCRT complex mutations have pronounced proliferation, loss of cell polarity, apoptosis activation and other developmental functions. (17, 18). In Caenorhabditis elegans specifically, mutations in all the ESCRT proteins present an accumulation of abnormal endosomes and autophagosomes (19). ESCRT mutants of yeast display an amassing of hydrolases and endocytosed receptors in abnormally enlarged endosomes.

Potential target genes may be identified based on homologies to genes in other insect species. Published genome-wide RNAi mediated gene interference libraries (15, 16) may be used to identify genes that are lethal to other organisms when RNAi based on these genes is expressed and incorporated into target pest organisms by ingestion or any other means. Thus genes identified as being RNAi-lethal in Drosophila or western corn rootworm Diabrotica virgifera virgifera LeConte may be used to screen for orthologs in hymenoptera species. Such hymenoptera orthologs may further be used to screen gall wasp species for potential targets.

Li and Om are sessile pests. Accordingly, Li and Om vital target genes cannot be predicted solely on the basis of genes that were shown to be vital genes in a non-sessile pest. Sessile pests, for example, cannot migrate to an alternative feed source. In the case of Li and Om, developing pests are confined to the gall and during an 80-120 day period feed on the same source. This mode of development results in the possibility that slow but continuous uptake of dsRNA can have a cumulative effect that would not be effective in a non-sessile pest.

Examples of target genes include, without limitation Multivesticular body subunit 12B-like (Dmel); NADH dehydrogenase [ubiquinone] iron sulfur protein 7 (Vps23); Vacuolar Protein Sorting-Associated Protein 28 homolog (Vps28); Vacuolar protein sorting associated protein 37A like (Vps37/mod-r); Vacuolar protein sorting associated protein 37B like (Vps37b); Vacuolar sorting protein SNF8 like (Vps22/Isn); Vacuolar protein sorting associated protein 25 like (Vps 25); Vacuolar protein sorting associated protein 36 (Vps36); Charged Multivesicular Body Protein 2a like (Vps2); Charged multivesicular body protein 6 like (Vps20); Charged multivesicular body protein 3 like (Vps24); and Charged Multivesicular Body Protein 4b like (Snf7/shrub), SWI/SNF complex subunit SMARCC2 (MOR) and eukaryotic translation initiation factor 3 subunit I-like protein (TIF). Nucleotide sequences of gall wasp target genes include, for example, the sequences set out in SEQ ID NO: 1-54 the complements of such sequences, and sequences that selectively hybridize to such sequences and complements under high stringency hybridization conditions.

Nucleotide sequences useful for dsRNA-mediated downregulation of gall wasp target genes include, for example, (i) sequences set out in SEQ ID NO: 1-54 and 70-74 and the complements of such sequences; (ii) sequences which are at least 70%, 75%, 80%, 85%, 90%, 95%, 96%, 97%, 98%, 99% or 99.9% identical to a sequence set out in SEQ ID NO: 1-54 and the complements of such sequences; (iii) sequences comprising at least 17 contiguous nucleotides of SEQ ID NO: 1-54 and 70-74 and the complements of such sequences; and (iv) sequences that selectively hybridize to such sequences and complements under high stringency hybridization conditions.

An “isolated” nucleic acid as used herein is a nucleic that has been identified and separated and/or recovered from a component of its natural environment.

“Controlling pests” as used herein means killing pests, or preventing pests to develop, or to grow or preventing pests to infect or infest. Controlling pests as used herein also encompasses controlling pest progeny (development of eggs). Controlling pests as used herein also encompasses inhibiting viability, growth, development or reproduction of the pest, or to decrease pathogenicity or infectivity of the pest. The compounds and/or compositions described herein, may be used to keep an organism healthy and may be used curatively, preventively or systematically to control pests or to avoid pest growth or development or infection or infestation.

Particular pests envisaged for control by methods described herein are plant pathogenic insect pests. “Controlling insects” as used herein thus encompasses controlling insect progeny (such as development of eggs). Controlling insects as used herein also encompasses inhibiting viability, growth, development or reproduction of the insect, or decreasing pathogenicity or infectivity of the insect. As used herein, controlling insects may refer to inhibiting a biological activity in an insect, resulting in one or more of the following attributes: reduction in feeding by the insect, reduction in viability of the insect, death of the insect, inhibition of differentiation and development of the insect, absence of or reduced capacity for sexual reproduction by the insect.

The compounds and/or compositions described herein, may be used to keep an organism healthy and may be used curatively, preventively or systematically to control an insect or to avoid insect growth or development or infection or infestation. Thus, the invention may allow previously susceptible organisms to develop resistance against infestation by the insect organism.

The term “complementary to at least part of” refers to a nucleotide sequence that is fully complementary to the nucleotide sequence of the target over more than ten nucleotides, for instance over at least 15, 16, 17, 18, 19, 20, 21, 22, 23, 24 or more contiguous nucleotides. Notwithstanding the above, “complementary to at least part” of may also include complementary sequences that are greater than 80% complementary to a nucleotide sequence of a target sequence over a length of more than 20 nucleotides, for instance over at least 20, 21, 22, 23, 24 or more contiguous nucleotides [13, 14].

In certain aspects, the invention provides a method for down-regulating expression of a target gene in an insect, comprising contacting the insect with a dsRNA, wherein the dsRNA comprises annealed complementary strands, one of which has a nucleotide sequence that is complementary to at least part of the nucleotide sequence of the insect target gene to be down-regulated, whereby the dsRNA is taken up into the insect and thereby down-regulates expression of the insect target gene.

The term “insect” encompasses insects of all types and at all stages of development, including egg, larval or nymphal, pupal and adult stages.

As used herein, the term “plant” encompasses any plant material that it is desired to treat to prevent or reduce insect growth and/or insect infestation. This includes, inter alia, whole plants, seedlings, propagation or reproductive material such as seeds, cuttings, grafts, explants, etc., and also plant cell and tissue cultures. The plant material should express, or have the capability to express, the RNA molecule comprising at least one nucleotide sequence that is the RNA complement of or that represents the RNA equivalent of at least part of the nucleotide sequence of the sense strand of at least one target gene of the pest organism, such that the RNA molecule is taken up by a pest upon plant-pest interaction, said RNA molecule being capable of inhibiting the target gene or down-regulating expression of the target gene by RNA interference.

The terms “down-regulation of gene expression” and “inhibition of gene expression” are used interchangeably and refer to a measurable or observable reduction in gene expression or a complete abolition of detectable gene expression, at the level of protein product and/or mRNA product from the target gene. The down-regulation effect of the dsRNA on gene expression may be calculated as being at least 30%, 40%, 50%, 60%, preferably 70%, 80% or even more preferably 90% or 95% when compared with normal gene expression. Depending on the nature of the target gene, down-regulation or inhibition of gene expression in cells of an insect can be confirmed by phenotypic analysis of the cell or the whole insect or by measurement of mRNA or protein expression using molecular techniques such as RNA solution hybridization, PCR, nuclease protection, Northern hybridization, reverse transcription, gene expression monitoring with a microarray, antibody binding, enzyme-linked immunosorbent assay (ELISA), Western blotting, radioimmunoassay (RIA), other immunoassays, or fluorescence-activated cell analysis (FACS).

Down-regulation of an essential gene leads to growth inhibition. Depending on the assay used, the growth inhibition can be quantified as being greater than about 5%, 10%, more preferably about 20%, 25%, 33%, 50%, 60%, 75%, 80%, most preferably about 90%, 95%, or about 99% as compared to a pest organism that has been treated with control dsRNA.

The “target gene” may be essentially any gene that is desirable to be inhibited because it interferes with growth or pathogenicity or infectivity of the insect. For instance, if the method of the invention is to be used to prevent insect growth and/or infestation then it is preferred to select a target gene which is essential for viability, growth, development or reproduction of the insect, or any gene that is involved with pathogenicity or infectivity of the insect, such that specific inhibition of the target gene leads to a lethal phenotype or decreases or stops insect infestation.

According to one non-limiting embodiment, the target gene is such that when its expression is down-regulated or inhibited using the method of the invention, the insect is killed, or the reproduction or growth of the insect is stopped or retarded. This type of target gene is considered to be essential for the viability of the insect and is referred to as essential genes. Therefore, the present invention encompasses a method as described herein, wherein the target gene is an essential gene.

Without being bound by theory, the target gene is such that when it is down-regulated the infestation or infection by the insect, the damage caused by the insect, and/or the ability of the insect to infest or infect host organisms and/or cause such damage, is reduced. The terms “infest” and “infect” or “infestation” and “infection” are generally used interchangeably throughout. This type of target gene is considered to be involved in the pathogenicity or infectivity of the insect. Therefore, the present invention extends to methods as described herein, wherein the target gene is involved in the pathogenicity or infectivity of the insect. The advantage of choosing the latter type of target gene is that the insect is blocked to infect further plants or plant parts and is inhibited to form further generations.

In dsRNA-mediated methods of controlling growth or infestation of a specific insect in or on a host cell or host organism, it is preferred that the dsRNA does not share any significant homology with any host gene, or at least not with any essential gene of the host. In this context, it is preferred that the dsRNA shows less than 30%, more preferably less that 20%, more preferably less than 10%, and even more preferably less than 5% nucleic acid sequence identity with any gene of the host cell. Percent sequence identity should be calculated across the full length of the dsRNA region. If genomic sequence data is available for the host organism one may cross-check sequence identity with the dsRNA using standard bioinformatics tools. In one embodiment, there is no sequence identity between the dsRNA and a host sequences over 21 contiguous nucleotides, meaning that in this context, it is preferred that 21 contiguous base pairs of the dsRNA do not occur in the coding sequences (CDS) of the host organism. In another embodiment, there is less than about 10% or less than about 12.5% sequence identity over 24 contiguous nucleotides of the dsRNA with any nucleotide sequence from a host species.

dsRNA comprises annealed complementary strands, one of which has a nucleotide sequence which corresponds to a target nucleotide sequence of the target gene to be down-regulated. The other strand of the dsRNA is able to base-pair with the first strand.

The expression “target region” or “target nucleotide sequence” of the target insect gene may be any suitable region or nucleotide sequence of the gene. The target region should comprise at least 17, at least 18 or at least 19 consecutive nucleotides of the target gene, more preferably at least 20 or at least 21 nucleotide and still more preferably at least 22, 23 or 24 nucleotides of the target gene.

It is preferred that (at least part of) the dsRNA will share 100% sequence identity with the target region of the insect target gene. However, it will be appreciated that 100% sequence identity over the whole length of the double stranded region is not essential for functional RNA inhibition. RNA sequences with insertions, deletions, and single point mutations relative to the target sequence have also been found to be effective for RNA inhibition.

The terms “corresponding to” or “complementary to” are used herein interchangeably, and when these terms are used to refer to sequence correspondence between the dsRNA and the target region of the target gene, they are to be interpreted accordingly, i.e., as not absolutely requiring 100% sequence identity. However, the percent sequence identity between the dsRNA and the target region will generally be at least 80% or 85% identical, preferably at least 90%, 95%, 96%, or more preferably at least 97%, 98% and still more preferably at least 99%. Two nucleic acid strands are “substantially complementary” when at least 85% of their bases pair.

The term “complementary” as used herein relates to all of DNA-DNA complementarity, RNA-RNA complementarity and to DNA-RNA complementarity. In analogy herewith, the term “RNA equivalent” substantially means that in the DNA sequence(s), the base “T” may be replaced by the corresponding base “U” normally present in ribonucleic acids.

Although dsRNA contains a sequence which corresponds to the target region of the target gene, it is not essential for the whole of the dsRNA to correspond to the sequence of the target region. For example, the dsRNA may contain short non-target regions flanking the target-specific sequence, provided that such sequences do not affect performance of the dsRNA in RNA inhibition to a material extent.

The dsRNA may contain one or more substitute bases in order to optimize performance in RNAi. It will be apparent to one of ordinary skill in the art how to vary each of the bases of the dsRNA in turn and test the activity of the resulting dsRNAs (e.g., in a suitable in vitro test system) in order to optimize the performance of a given dsRNA.

The dsRNA may further contain DNA bases, non-natural bases or non-natural backbone linkages or modifications of the sugar-phosphate backbone, for example to enhance stability during storage or enhance resistance to degradation by nucleases.

Interfering RNAs (siRNAs) of about 21 bp are useful for effective gene silencing. Increasing the length of dsRNA preferably to at least about 80-100 bp may increase the efficiency by which dsRNA is taken up by pest organisms. Such longer fragments may be more effective in gene silencing, possibly due to a more efficient uptake of these long dsRNA by the invertebrate.

RNA duplexes consisting of either 27-mer blunt or short hairpin (sh) RNAs with 29 bp stems and 2-nt 3′ overhangs may also be used as siRNAs. Thus, molecules based upon the targets identified above and being either 27-mer blunt or short hairpin (sh) RNA's with 29-bp stems and 2-nt 3′ overhangs are also included within the scope of the invention.

Therefore, in one embodiment, the dsRNA fragment (or region) will itself preferably be at least 17 bp in length, preferably 18 or 19 bp in length, more preferably at least 20 bp, more preferably at least 21 bp, or at least 22 bp, or at least 23 bp, or at least 24 bp, 25 bp, 26 bp or at least 27 bp in length. The expressions “double-stranded RNA fragment” or “double-stranded RNA region” refer to a small entity of the dsRNA corresponding with (part of) the target gene.

More generally, the double stranded RNA is preferably between about 17-1500 bp, even more preferably between about 80-1000 bp and most preferably between about 17-27 bp or between about 80-250 bp; such as double stranded RNA regions of about 17 bp, 18 bp, 19 bp, 20 bp, 21 bp, 22 bp, 23 bp, 24 bp, 25 bp, 27 bp, 50 bp, 80 bp, 100 bp, 150 bp, 200 bp, 250 bp, 300 bp, 350 bp, 400 bp, 450 bp, 500 bp, 550 bp, 600 bp, 650 bp, 700 bp, 900 bp, 100 bp, 1100 bp, 1200 bp, 1300 bp, 1400 bp or 1500 bp.

The upper limit on the length of the dsRNA may be dependent on i) the requirement for the dsRNA to be taken up by the insect and ii) the requirement for the dsRNA to be processed within the cell into fragments that direct RNAi. The chosen length may also be influenced by the method of synthesis of the RNA and the mode of delivery of the RNA to the cell. Preferably the dsRNA to be used in the methods of the invention will be less than 10,000 bp in length, more preferably 1000 bp or less, more preferably 500 bp or less, more preferably 300 bp or less, more preferably 100 bp or less. For any given target gene and insect, the optimum length of the dsRNA for effective inhibition may be determined by experiment.

The dsRNA may be fully or partially double-stranded. Partially dsRNAs may include short single-stranded overhangs at one or both ends of the double-stranded portion, provided that the RNA is still capable of being taken up by insects and directing RNAi. The dsRNA may also contain internal non-complementary regions.

The methods of the invention encompass the simultaneous or sequential provision of two or more different dsRNAs or RNA constructs to the same insect, so as to achieve down-regulation or inhibition of multiple target genes or to achieve a more potent inhibition of a single target gene.

Alternatively, multiple targets are hit by the provision of one dsRNA that hits multiple target sequences, and a single target is more efficiently inhibited by the presence of more than one copy of the double stranded RNA fragment corresponding to the target gene. Thus, in certain aspects, a dsRNA construct comprises multiple dsRNA regions, at least one strand of each dsRNA region comprising a nucleotide sequence that is complementary to at least part of a target nucleotide sequence of an insect target gene. The dsRNA regions in the RNA construct may be complementary to the same or to different target genes and/or the dsRNA regions may be complementary to targets from the same or from different insect species.

The terms “hit”, “hits” and “hitting” are alternative wordings to indicate that at least one of the strands of the dsRNA is complementary to, and as such may bind to, the target gene or nucleotide sequence.

In one embodiment, the double stranded RNA region comprises multiple copies of the nucleotide sequence that is complementary to the target gene. Alternatively, the dsRNA hits more than one target sequence of the same target gene. The invention thus encompasses isolated double stranded RNA constructs comprising at least two copies of said nucleotide sequence complementary to at least part of a nucleotide sequence of an insect target.

The term “multiple” as used herein means at least two, at least three, at least four, at least five, at least six, etc.

The expressions “a further target gene” or “at least one other target gene” mean for instance a second, a third or a fourth, etc. target gene.

dsRNA that hits more than one of the above-mentioned targets, or a combination of different dsRNA against different of the above mentioned targets are developed and used in the methods of the present invention.

dsRNA regions (or fragments) in the double stranded RNA may be combined as follows: a) when multiple dsRNA regions targeting a single target gene are combined, they may be combined in the original order (i.e., the order in which the regions appear in the target gene) in the RNA construct; b) alternatively, the original order of the fragments may be ignored so that they are scrambled and combined randomly or deliberately in any order into the double stranded RNA construct; c) alternatively, one single fragment may be repeated several times, for example from 1 to 10 times, e.g., 1, 2, 3, 4, 5, 6, 7, 8, 9 or 10 times, in the ds RNA construct, or d) the dsRNA regions (targeting a single or different target genes) may be combined in the sense or antisense orientation.

Multiple dsRNA regions targeting a single or different weak gene(s) may be combined to obtain a stronger RNAi effect. “Insect specific” genes or sequences, e.g., gall wasp specific, particularly Li or Om specific genes and sequences, encompass genes that have no substantial homologous counterpart in non-insect organisms as can be determined by bioinformatics homology searches, for example by BLAST searches. The choice of a specific target gene results in a species specific RNAi effect, with no effect or no substantial (adverse) effect in non-target organisms. “Conserved genes” encompass genes that are conserved (at the amino acid level) between the target organism and non-target organism(s). To reduce possible effects on non-target species, such effective but conserved genes are analyzed and target sequences from the variable regions of these conserved genes are chosen to be targeted by the dsRNA regions in the RNA construct. Conservation is assessed at the level of the nucleic acid sequence. Such variable regions thus encompass the least conserved sections, at the level of the nucleic acid sequence, of the conserved target gene(s). The RNA constructs according to the present invention target multiple genes from different biological pathways, resulting in a broad cellular RNAi effect and more efficient insect control. In certain embodiments dsRNAs are constructed from sequences, e.g., Li and Om transcriptome sequences, that are equal to or less than 80% identical to the sequence of a honey bee ortholog. In other embodiments dsRNAs are constructed from sequences, e.g., Li and Om ESCRT family protein sequences, that are equal to or less than 80% identical to the sequence of a western corn worm Diabrotica virgifera virgifera LeConte ortholog, for example and without limitation, the western corn worm Diabrotica virgifera virgifera LeConte ortholog of the Li and Om sequences.

In certain aspects, dsRNA constructs are constructed with gene sequences that affect different classes of cellular functions. Examples of such classes of cellular function include, without limitation, (i) protein synthesis and metabolism, (ii) RNA synthesis and metabolism, and (iii) cellular processes, including without limitation multivesticular body biogenesis and endosomal cargo sorting. In certain embodiments, dsRNA constructs comprise sequences from each of the aforementioned claims, i.e., three classes. In certain embodiments, dsRNA constructs comprise sequences from two of the aforementioned classes, e.g., protein synthesis and metabolism and RNA synthesis and metabolism; protein synthesis and cellular processes; or RNA synthesis and metabolism and cellular processes.

dsRNA regions comprise at least one strand that is complementary to at least part or a portion of the nucleotide sequence of any of the target genes herein described. However, provided one of the double stranded RNA regions comprises at least one strand that is complementary to a portion of the nucleotide sequence of any one of the target genes herein described, the other double stranded RNA regions may comprise at least one strand that is complementary to a portion of any other insect target gene (including known target genes).

In some constructs, dsRNAs may comprise additional sequences and optionally a linker. Additional sequences may include, for example, (i) a sequence facilitating large-scale production of the dsRNA construct; (ii) a sequence effecting an increase or decrease in the stability of the dsRNA; (iii) a sequence allowing the binding of proteins or other molecules to facilitate uptake of the RNA construct by insects; (iv) a sequence which is an aptamer that binds to a receptor or to a molecule on the surface or in the cytoplasm of an insect to facilitate uptake, endocytosis and/or transcytosis by the insect; or (v) additional sequences to catalyze processing of dsRNA regions. In one embodiment, the linker is a conditionally self-cleaving RNA sequence, preferably a pH sensitive linker or a hydrophobic sensitive linker.

Multiple dsRNA regions of the dsRNA construct may be connected directly or by one or more linkers. A linker may be present at a site in the RNA construct, separating dsRNA regions from another region of interest. Multiple dsRNA regions of dsRNA constructs may be connected without linkers.

When present, linkers may be used to disconnect smaller dsRNA regions in the pest organism. Advantageously, in this situation the linker sequence may promote division of a long dsRNA into smaller dsRNA regions under particular circumstances, resulting in the release of separate dsRNA regions under these circumstances and leading to more efficient gene silencing by these smaller dsRNA regions. Examples of suitable conditionally self-cleaving linkers are RNA sequences that are self-cleaving at high pH conditions. Suitable examples of such RNA sequences are described by Borda et al. (Nucleic Acids Res. 2003 May 15; 31(10):2595-600), which document is incorporated herein by reference. This sequence originates from the catalytic core of the hammerhead ribozyme HH16.

Linkers may also be located at a site in the dsRNA construct, separating the dsRNA regions from another, e.g., an additional, sequence of interest, which preferably provides some additional function to the RNA construct.

dsRNA constructs may include aptamers to facilitate uptake of the dsRNA by the insect. The aptamer is designed to bind a substance which is taken up by the insect. Such substances may be from an insect or plant origin. One specific example of an aptamer, is an aptamer that binds to a transmembrane protein, for example a transmembrane protein of an insect. Alternatively, the aptamer may bind a (plant) metabolite or nutrient which is taken up by the insect.

Linkers may undergo self-cleaving in the endosome. This may be advantageous when the constructs of the present invention are taken up by the insect via endocytosis or transcytosis, and are therefore compartmentalized in the endosomes of the insect species. The endosomes may have a low pH environment, leading to cleavage of the linker.

Linkers that are self-cleaving in hydrophobic conditions are particularly useful in dsRNA constructs when used to be transferred from one cell to another via the transit in a cell wall, for example when crossing the cell wall of an insect pest organism.

An intron may be used as a linker. An “intron” as used herein may be any non-coding RNA sequence of a messenger RNA.

A non-complementary RNA sequence, ranging from about 1 base pair to about 10,000 base pairs, may also be used as a linker.

Without wishing to be bound by any particular theory or mechanism, it is thought that long dsRNAs are taken up by the insect from their immediate environment. dsRNAs taken up into the gut and transferred to the gut epithelial cells are then processed within the cell into short dsRNAs, called small interfering RNAs (siRNAs), by the action of an endogenous endonuclease. The resulting siRNAs then mediate RNAi via formation of a multi-component RNase complex termed the RISC or RNA interfering silencing complex.

In order to achieve down-regulation of a target gene within an insect cell the dsRNA added to the exterior of the cell wall may be any dsRNA or dsRNA construct that can be taken up into the cell and then processed within the cell into siRNAs, which then mediate RNAi, or the RNA added to the exterior of the cell could itself be an siRNA that can be taken up into the cell and thereby direct RNAi.

siRNAs are generally short dsRNAs having a length in the range of from 19 to 25 base pairs, or from 20 to 24 base pairs. In preferred embodiments siRNAs having 19, 20, 21, 22, 23, 24 or 25 base pairs, and in particular 21 or 22 base pairs, corresponding to the target gene to be down-regulated may be used. However, the invention is not intended to be limited to the use of such siRNAs.

siRNAs may include single-stranded overhangs at one or both ends, flanking the double-stranded portion. The siRNA may contain 3′ overhanging nucleotides, preferably two 3′ overhanging thymidines (dTdT) or uridines (UU). 3′ TT or UU overhangs may be included in the siRNA if the sequence of the target gene immediately upstream of the sequence included in double-stranded part of the dsRNA is AA. This allows the TT or UU overhang in the siRNA to hybridize to the target gene. Although a 3′ TT or UU overhang may also be included at the other end of the siRNA it is not essential for the target sequence downstream of the sequence included in double-stranded part of the siRNA to have AA. In this context, siRNAs which are RNA/DNA chimeras are also contemplated. These chimeras include, for example, the siRNAs comprising a dsRNA with 3′ overhangs of DNA bases (e.g., dTdT), as discussed above, and also dsRNAs which are polynucleotides in which one or more of the RNA bases or ribonucleotides, or even all of the ribonucleotides on an entire strand, are replaced with DNA bases or deoxyribonucleotides.

dsRNA may be formed from two separate (sense and antisense) RNA strands that are annealed together by (non-covalent) base pairing. Alternatively, the dsRNA may have a foldback stem-loop or hairpin structure, wherein the two annealed strands of the dsRNA are covalently linked. In this embodiment the sense and antisense stands of the dsRNA are formed from different regions of single polynucleotide molecule that is partially self-complementary. RNAs having this structure are convenient if the dsRNA is to be synthesized by expression in vivo, for example in a host cell or organism, or by in vitro transcription. The precise nature and sequence of the “loop” linking the two RNA strands is generally not material to the invention, except that it should not impair the ability of the double-stranded part of the molecule to mediate RNAi. The features of “hairpin” or “stem-loop” RNAs for use in RNAi are generally known in the art (see for example WO 99/53050, the contents of which are incorporated herein by reference). In other embodiments of the invention, the loop structure may comprise linker sequences or additional sequences as described above.

In certain aspects, the Li and Om sequences disclosed herein and the complements of such sequences may also be used to inhibit expression of Li or Om nucleic acids via expression of antisense RNA or overexpression of sense RNA, using methods well known in the art. See, e.g., Frizzi et al., Plant Biotech J, (2010) 8:655-677; Brodersen et al., Trends in Genetics, (2008) 22:268-280; and U.S. Pat. No. 5,759,829. Using expression elements, vectors and methods described herein, antisense RNAs or sense RNAs for Li and Om target genes are expressed in eucalyptus plants. Upon ingestion by Om or Li pests, the antisense or sense RNAs inhibit expression of the target genes to control pest infestation.

Target nucleotide sequences for design the dsRNA constructs are preferably at least 17, preferably at least 18, 19, 20 or 21, more preferably at least 22, 23 or 24 nucleotides in length. Non-limiting examples of preferred target nucleotide sequences are given in the examples.

Target sequences may include sequences that are homologous to sequences disclosed herein. Homologues of target genes can be found using methods well known to those of ordinary skill in the art. Preferred homologues are genes comprising a sequence which is at least about 85% or 87.5%, still more preferably about 90%, still more preferably at least about 95% and most preferably at least about 99% or 99.9% identical to a sequence disclosed herein, or the complement thereof. Methods for determining sequence identity are routine in the art and include use of the Blast software and EMBOSS software (The European Molecular Biology Open Software Suite (2000), Rice, P. Longden, I. and Bleasby, A. Trends in Genetics 16, (6) pp 276-277). The term “identity” as used herein refers to the relationship between sequences at the nucleotide level. The expression “% identical” is determined by comparing optimally aligned sequences, e.g., two or more, over a comparison window wherein the portion of the sequence in the comparison window may comprise insertions or deletions as compared to the reference sequence for optimal alignment of the sequences. The reference sequence does not comprise insertions or deletions. The reference window is chosen from between at least 10 contiguous nucleotides to about 50, about 100 or to about 150 nucleotides, preferably between about 50 and 150 nucleotides. “percent identity” is then calculated by determining the number of nucleotides that are identical between the sequences in the window, dividing the number of identical nucleotides by the number of nucleotides in the window and multiplying by 100.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ, can be approximated from the equation of Meinkoth and Wahl, (1984) Anal. Biochem., 138:267-84: Tₘ=81.5° C.+16.6 (log M)+0.41 (% GC)-0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ, is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ, can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used.

An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, N.Y. (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

dsRNA may be expressed by (e.g., transcribed within) a host cell or host organism. The host cell or organism may or may not be a host cell or organism susceptible or vulnerable to infestation by an insect. If the host cell or organism is a host cell or organism susceptible or vulnerable to infestation by an insect, RNAi-mediated gene silencing of one or more target genes in the insect may be used as a mechanism to control growth of the insect in or on the host organism and/or to prevent or reduce insect infestation of the host organism. Expression of the dsRNA within cells of the host organism may thus confer resistance to a particular insect or to a class of insects. In case the dsRNA hits more than one insect target gene, expression of the dsRNA within cells of the host organism may confer resistance to more than one insect or more than one class of insects.

In a preferred embodiment the host organism is a plant and the insect is a plant pathogenic insect. In this embodiment the insect is contacted with the dsRNA by expressing the dsRNA in a plant, plant tissue or plant cell that is infested with or susceptible to infestation with, or ingestion by, the plant pathogenic insect. A preferred plant host organism is eucalyptus. Examples of eucalyptus include, without limitation, the following species: E. botryoides, E. bridgesiana, E. camaldulensis, E. cinerea, E. globule, E. grandis, E. gunii, E. nicholii, E. pulverulenta, E. robusta, E. rudis, E. saligna, E. Tereticornis, E. Urophilla, E. viminalis and a cross hybrids of any of the preceding species especially Eucalyptus grandis and Eucalyptus urophylla. A preferred plant pathogenic insect is a gall wasp, e.g., Li or Om.

The term “plant” encompasses any plant material that it is desired to treat to prevent or reduce insect growth and/or insect infestation. This includes, inter alia, whole plants, seedlings, propagation or reproductive material such as seeds, cuttings, grafts, explants, etc. and also plant cell and tissue cultures. The plant material should express, or have the capability to express, dsRNA corresponding to one or more target genes of the insect.

In certain aspects the invention provides a plant, preferably a transgenic plant, or propagation or reproductive material for a (transgenic) plant, or a plant cell culture expressing or capable of expressing at least one dsRNA, wherein the dsRNA comprises annealed complementary strands, one of which has a nucleotide sequence which is complementary to at least part of a target nucleotide sequence of a target gene of an insect, such that the dsRNA is taken up by an insect upon plant-insect interaction, said double stranded RNA being capable of inhibiting the target gene or down-regulating expression of the target gene by RNA interference. The target gene may be any of the target genes herein described, for instance a target gene that is essential for the viability, growth, development or reproduction of the insect.

A plant may be provided in a form that is actively expressing (transcribing) a dsRNA in one or more cells, cell types or tissues. Alternatively, a plant may be “capable of expressing”, meaning that it is transformed with a transgene which encodes the desired dsRNA but that the transgene is not active in the plant when (and in the form in which) the plant is supplied. A recombinant DNA construct comprising a nucleotide sequence encoding a dsRNA or dsRNA construct may be thus be operably linked to at least one regulatory sequence. Preferably, the regulatory sequence is selected from the group comprising constitutive promoters or tissue specific promoters as described below.

A target gene may be any target gene herein described. Preferably a regulatory element is a regulatory element that is active in a plant cell. More preferably, the regulatory element is originating from a plant. The term “regulatory sequence” is to be taken in a broad context and refers to a regulatory nucleic acid capable of effecting expression of the sequences to which it is operably linked.

Encompassed by the aforementioned term are promoters and nucleic acids or synthetic fusion molecules or derivatives thereof which activate or enhance transcription of a nucleic acid, so called activators or enhancers. The term “operably linked” as used herein refers to a functional linkage between the promoter sequence and the gene of interest, such that the promoter sequence is able to initiate transcription of the gene of interest.

By way of example, the transgene nucleotide sequence encoding the dsRNA could be placed under the control of an inducible or growth or developmental stage-specific promoter which permits transcription of the dsRNA to be turned on, by the addition of the inducer for an inducible promoter or when the particular stage of growth or development is reached.

Alternatively, the transgene encoding the dsRNA is placed under the control of a strong constitutive promoter such as any selected from the group comprising the CaMV35S promoter, doubled CaMV35S promoter, ubiquitin promoter, actin promoter, rubisco promoter, GOS2 promoter, Figwort mosaic virus (FMV) 34S promoter, cassaya vein mosaic virus (CsVMV) promoter (Verdaguer B. et al, Plant Mol. Biol. 1998 37(6):1055-67).

Alternatively, the transgene encoding the dsRNA is placed under the control of a tissue specific promoter such as any selected from the group comprising root specific promoters of genes encoding PsMTA Class III chitinase, photosynthetic tissue-specific promoters such as promoters of cab1 and cab2, rbcS, gapA, gapB and ST-LS1 proteins, JAS promoters, chalcone synthase promoter and promoter of RJ39 from strawberry.

A transgene encoding the dsRNA may also be placed under the control of an insect-induced promoter, for instance the potato proteinase inhibitor II (PinII) promoter (Duan X et al, Nat. Biotechnol. 1996, 14(4):494-8)); or a wounding-induced promoter, for instance the jasmonates and ethylene induced promoters, PDF1.2 promoter (Manners J M et al., Plant Mol. Biol. 1998, 38(6):1071-80); or under a defense related promoter, for instance the salicylic acid induced promoters and plant-pathogenesis related protein (PR protein) promoters (PR1 promoter (Cornelissen B J et al., Nucleic Acids Res. 1987, 15(17):6799-811; COMT promoter (Toquin V et al, Plant Mol. Biol. 2003, 52(3):495-509).

When using the methods described herein for developing transgenic plants resistant against insects, it may be beneficial to place the nucleic acid encoding the dsRNA under the control of a tissue-specific promoter. In order to improve the transfer of the dsRNA from the plant cell to the pest, the plants could preferably express the dsRNA in a plant part that is first accessed or damaged by the plant pest. In case of plant pathogenic insects, preferred tissues to express the dsRNA are the leaves, stems, roots, and seeds. Therefore, in the methods disclosed herein, a plant tissue-preferred promoter may be used, such as a leaf-specific promoter, a stem-specific promoter, a phloem-specific promoter, a xylem-specific promoter, a root-specific promoter, or a seed-specific promoter (sucrose transporter gene AtSUC promoter (Baud S et al., Plant J. 2005, 43(6):824-36), wheat high molecular weight glutenin gene promoter (Robert L S et al., Plant Cell. 1989, 1(6):569-78.)).

Suitable examples of a root specific promoter are PsMTA (Fordam-Skelton, A. P., et al., 1997 Plant Molecular Biology 34: 659-668.) and the Class III Chitinase promoter. Examples of leaf- and stem-specific or photosynthetic tissue-specific promoters that are also photoactivated are promoters of two chlorophyll binding proteins (cab1 and cab2) from sugar beet (Stahl D. J., et al., 2004 BMC Biotechnology 2004 4:31), ribulose-bisphosphate carboxylase (Rubisco), encoded by rbcS (Nomura M. et al., 2000 Plant Mol. Biol. 44: 99-106), A (gapA) and B (gapB) subunits of chloroplast glyceraldehyde-3-phosphate dehydrogenase (Conley T. R. et al. 1994 Mol. Cell. Biol. 19: 2525-33; Kwon H. B. et al. 1994 Plant Physiol. 105: 357-67), promoter of the Solanum tuberosum gene encoding the leaf and stem specific (ST-LS1) protein (Zaidi M. A. et al., 2005 Transgenic Res. 14:289-98), stem-regulated, defense-inducible genes, such as JAS promoters (patent publication no. 20050034192/US-A1). An example of a flower-specific promoter is for instance, the chalcone synthase promoter (Faktor 0. et al. 1996 Plant Mol. Biol. 32: 849) and an example of a fruit-specific promoter is for instance RJ39 from strawberry (WO 98 31812).

Other promoters useful for the expression of dsRNA are used and include, but are not limited to, promoters from an RNA Poll, an RNA Poll, an RNA PolIII, T7 RNA polymerase or SP6 RNA polymerase. These promoters are typically used for in vitro-production of dsRNA, which dsRNA is then included in an anti-insecticidal agent, for example, in an anti-insecticidal liquid, spray or powder.

The dsRNA or RNA constructs described herein may be generated by the steps of (i) contacting an isolated nucleic acid or a recombinant DNA construct with cell-free components; or (ii) introducing (e.g., by transformation, transfection or injection) an isolated nucleic acid or a recombinant DNA construct into a cell, under conditions that allow transcription of the nucleic acid or recombinant DNA construct to produce the dsRNA or RNA construct.

Optionally, one or more transcription termination sequences may also be incorporated in the recombinant construct. The term “transcription termination sequence” encompasses a control sequence at the end of a transcriptional unit, which signals 3′ processing and poly-adenylation of a primary transcript and termination of transcription. Additional regulatory elements, such as transcriptional or translational enhancers, may be incorporated in the expression construct.

Recombinant constructs may further include an origin of replication which is required for maintenance and/or replication in a specific cell type. One example is when an expression construct is required to be maintained in a bacterial cell as an episomal genetic element (e.g., plasmid or cosmid molecule) in a cell. Preferred origins of replication include, but are not limited to, fl-ori and colE1 ori.

Recombinant construct may optionally include a selectable marker gene. As used herein, the term “selectable marker gene” includes any gene, which confers a phenotype on a cell in which it is expressed to facilitate the identification and/or selection of cells, which are transfected or transformed, with an expression construct of the invention. Examples of suitable selectable markers include resistance genes against ampicillin (Amp^{r}), tetracycline (Tc^{r}), kanamycin (Kan^{r}), phosphinothricin, and chloramphenicol (CAT) gene. Other suitable marker genes provide a metabolic trait, for example manA. Visual marker genes may also be used and include for example beta-glucuronidase (GUS), luciferase and Green Fluorescent Protein (GFP).

Plants that have been stably transformed with a transgene encoding the dsRNA may be supplied as seed, reproductive material, propagation material or cell culture material which does not actively express the dsRNA but has the capability to do so. The plant may be provided in a form wherein it is actively expressing (transcribing) the RNA molecule in one or more cells, cell types or tissues. Alternatively, the plant may be “capable of expressing”, meaning that it is transformed with a transgene which encodes the desired RNA molecule but that the transgene is not active in the plant when (and in the form in which) the plant is supplied. Many vectors are available for this purpose, and selection of the appropriate vector will depend mainly on the size of the nucleic acid to be inserted into the vector and the particular host cell to be transformed with the vector.

General techniques for expression of exogenous dsRNA in plants for the purposes of RNAi are known in the art (see Baulcombe D, 2004, Nature. 431(7006):356-63. RNA silencing in plants, the contents of which are incorporated herein by reference). More particularly, methods for expression of dsRNA in plants for the purposes of down-regulating gene expression in plant pests such as nematodes or insects are also known in the art. Similar methods can be applied in an analogous manner in order to express dsRNA in plants for the purposes of down-regulating expression of a target gene in a plant pathogenic insect. In order to achieve this effect it is necessary only for the plant to express (transcribe) the dsRNA in a part of the plant which will come into direct contact with the insect, such that the dsRNA can be taken up by the insect. Depending on the nature of the insect and its relationship with the host plant, expression of the dsRNA could occur within a cell or tissue of a plant within which the insect is also present during its life cycle, or the RNA may be secreted into a space between cells, such as the apoplast, that is occupied by the insect during its life cycle. Furthermore, the dsRNA may be located in the plant cell, for example in the cytosol, or in the plant cell organelles such as a chloroplast, mitochondrion, vacuole or endoplastic reticulum.

During development, gall wasp larvae are exposed to the extracellular environment and to intracellular contents, due to ingestion (e.g., ingestion of apoplasts) or cell lysis. Accordingly, gall wasp larvae may be exposed to dsRNA that is either present in cells in the gall or that is secreted by cells in or around the gall.

Alternatively, the dsRNA may be secreted by the plant cell and by the plant to the exterior of the plant. As such, the dsRNA may form a protective layer on the surface of the plant.

In a further aspect, the invention also provides combinations of methods and compositions for preventing or protecting plants from pest infestation. For instance, one means provides using the plant transgenic approach combining methods using expression of dsRNA molecules and methods using expression of such Bt insecticidal proteins.

In a further embodiment, the invention relates to a composition for controlling insect growth and/or preventing or reducing insect infestation, comprising at least a plant part, plant cell, plant tissue or seed comprising at least one dsRNA, wherein said dsRNA comprises annealed complementary strands, one of which has a nucleotide sequence which is complementary to at least part of a nucleotide sequence of an insect target gene. Optionally, the composition further comprises at least one suitable carrier, excipient or diluent. The target gene may be any target gene described herein. Preferably the insect target gene is essential for the viability, growth, development or reproduction of the insect.

Whenever the term “a” is used within the context of “a target gene”, this means “at least one” target gene. The same applies for “a” target organism meaning “at least one” target organism, and “a” RNA molecule or host cell meaning “at least one” RNA molecule or host cell.

According to one embodiment, the methods of the invention rely on uptake by the insect of dsRNA present outside of the insect (e.g., by feeding) and does not require expression of dsRNA within cells of the insect. In addition, the present invention also encompasses methods as described above wherein the insect is contacted with a composition comprising the dsRNA. Gall wasp larvae typically do not feed outside the gall and it is thus preferable that gall wasp larvae are exposed to dsRNA via transgenic plant material.

The invention further provides a method for down-regulating expression of at least one target gene in a target organism (which is capable of ingesting a plant, plant part, plant cell or seeds) comprising feeding a plant, plant part, plant cell or seed to the target organism which plant, plant part, plant cell or seed expresses dsRNA.

In a more preferred aspect, the invention provides a method for down-regulating expression of at least one target gene in a target organism (which is capable of ingesting a host cell, or extracts thereof) comprising feeding a host plant, plant part, plant cell or seed to the target organism which host plant, plant part, plant cell or seed expresses a dsRNA molecule comprising a nucleotide sequence complementary to or representing the RNA equivalent of at least part of the nucleotide sequence of the at least one target gene, whereby the ingestion of the host cell, host plant, plant part, plant cell or seed by the target organism causes and/or leads to down-regulation of expression of the at least one target gene.

The invention provides for use of a plant, plant part, plant cell or seed as defined herein for down regulation of expression of an insect target gene. In more detailed terms, the invention provides for use of a host cell as defined herein and/or an RNA molecule comprising a nucleotide sequence that is the RNA complement of or that represents the RNA equivalent of at least part of the nucleotide sequence of a target gene from a target organism, as produced by transcription of a nucleic acid molecule in a plant, plant part, plant cell or seed, for instance in the manufacture of a commodity product, for down regulation of expression of a target gene.

According to one embodiment, the methods of the invention rely on a genetically modified organism (GMO) approach wherein the dsRNA is expressed by a cell or an organism infested with or susceptible to infestation by insects. Preferably, said cell is a plant cell or said organism is a plant.

For siRNA mediated downregulation of insect genes, dsRNA is introduced and/or expressed in an insect cell, either directly or indirectly. dsRNA can be added to an insect diet artificially or produced by a transgenic source of food such as bacteria and plants [2,8]. Transgenic plants transcribing inverted repeat RNAs comprised of insect gene specific sequences, can process it to dsRNA and later into siRNA (small interfering RNA that are the first product in the silencing pathway). Insects digesting such transgenic plants are affected by the plant synthesized dsRNA and siRNA [5]. This insect control method can be utilized to protect plants efficiently against specific pests [2,8]. It is not required, however, that dsRNA be processed to siRNA in plant material. dsRNA may be ingested by the insect pest and processed to siRNA for the first time within the insect cell.

Numerous methods for introducing foreign genes into plants are known and can be used to insert an NT polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. O. L. Gamborg & G. C. Phillips. Springer-Verlag Berlin Heidelberg N.Y., 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren & Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255; and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993; and Simpson, et al., (1986) Plant Mol. Biol. 6:403-15m all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application No. 604 662 A1 discloses a method for transforming monocots using Agrobacterium. European Application No. 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. Pat. Nos. 913,913 and 913,914, both filed Oct. 1, 1986, as referenced in U.S. Pat. No. 5,262,306, issued Nov. 16, 1993, the entire disclosures therein incorporated herein by reference.

Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731; and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

Following stable transformation, plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant.

Transformed plant may be regenerated by micropropagation which provides a rapid, consistent reproduction of the transformed plants. Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

In certain aspects the invention provides methods of producing a plant resistant to a plant pathogenic pest by transforming a plant cell with a recombinant DNA construct or combination of constructs that express a dsRNA; regenerating a plant from the transformed plant cell; and growing the transformed plant cell under conditions suitable for the expression said recombinant DNA construct.

The methods of the invention are applicable to gall wasp species, e.g., Leptocybe invasa (Li) and Ophelimus maskelli (Om) that are susceptible to gene silencing by RNA interference and that are capable of internalizing dsRNA from their immediate environment. The invention is applicable to the insect at any stage in its development. Because insects have a non-living exoskeleton, they cannot grow at a uniform rate and rather grow in stages by periodically shedding their exoskeleton. This process is referred to as molting or ecdysis. The stages between molts are referred to as “instars” and these stages may be targeted according to the invention. Also, insect eggs or live young may also be targeted according to the present invention. All stages in the developmental cycle, which includes metamorphosis in the pterygotes, may be targeted according to the present invention. Thus, individual stages such as larvae, pupae, nymph etc. stages of development may all be targeted.

Li and Om are pests for eucalyptus. The nucleic acids, dsRNAs and methods described herein are thus useful for treating or inhibiting Li and Om infection and infestation of eucalyptus.

In a preferred aspect, the invention provides RNAi mediated pest control to generate transgenic eucalyptus resistant to Gall wasps. Two eucalyptus gall wasp invasive species Leptocybe invasa (Li) and Ophelimus maskelli (Om) have recently spread out of Australia into plantations worldwide[6, 9]. Females can reproduce sexually or parthenogenetically and a full life cycle takes about 130 days. Eggs laid by these pests under the surface of tree veins and leaves induce the formation of galls in the target tissues that serve as a host shelter and food stock for the developing larvae. The exact compounds or signals that induce gall formation have not been elucidated. Thus secretions of compounds from the egg and larva but not from the mother are associated with gall wasp formation. Meristematic or omnipotent cells in young leaves are induced by either chemical, mechanical, viral or DNA manipulations which trigger their proliferation. The gall development parallels larvae development, thus the size and age of the gall can be correlated with the larvae developmental stage. Stone et al., 2002, “The population biology of oak gall wasps (Hymenoptera: Cynipidae)”. Annu. Rev. Entomol. 47:633-68. Larval maturation induces further development of the galls until pupal stage. When the adult gall wasps emerge they are able to fly away and lay hundreds of eggs, infecting both the same tree as well as close and remote trees during a period of 3-6 days before they die. Massive gall wasp attacks cause growth arrest, leaf defoliation and death resulting in potentially massive forest yield losses. The sessile developing larvae, which, feed on transgenic plant tissue producing RNAi active against vital genes in the larvae, are affected by a consistent and relatively long term uptake of the si/dsRNA molecules tailor designed to silent specific vital gall wasp genes. Ultimately the silencing effect leads to larval mortality in the early growth stage thus protecting the host and minimizing the size of the gall wasp population, protecting the host plant from the damage.

The larvae stage of gall wasps in non-transgenic trees can be as long as 130 days and thus the lethal effect of dsRNA can accumulate during the entire potential larvae growth phase. Once the larvae are dead, the development of the galls is arrested and the adult population is reduced, subsequently preventing infection on the same, neighboring or distal trees.

Transgenic resistant trees may be characterized by one or more of the following results on a “Gall Wasp Developmental Scale”:
1. No galls are developed.
2. Small galls (indicator of low larval mass measurements) are developed as compared to WT [maximum length, diameter, area and/or volume are measured].
3. Low larval mass measurements compared to WT: total larval weight reduced compared to WT
4. More galls are hosting dead larvae compared to WT.
5. More galls are hosting dead pupas compared to WT.
6. No adults are emerging from the gall or galls compared to WT.
7. Developmentally impaired adults lacking the ability to reproduce or spread compared to the adults grown on wild type trees compared to WT.

### EXAMPLES

### Example 1

### Gall Wasp Transcriptome Sequencing

Gall wasp infected leaves were collected from infected E. camaldulensis from Emek Izrael, Israel. Gall wasp larvae were removed from galls found on the leaves and or petioles of the trees by cutting and opening the galls using a sharp knife under a binocular microscope. A mixture of larval from various larval developmental stages were used. Batches of 100 larva was placed in a microtube on ice. The tube was then sealed and immediately frozen in liquid nitrogen and kept at −80° C. until further treatment. Total RNA was isolated using MasterPure RNA purification kit and protocol (MRC85102-Epicentere Biotechnologies). Total RNA volume was 50 μl. The total RNA was then treated with DNAse to remove any residual DNA remaining, followed by isolation of poly A mRNA (MicroPoly(A) Purist, Small scale mRNA Purification kit, AM1919 Ambion). mRNA final volume was 20 μl. The purified mRNA was kept at −80° C. until 454 Sequencing was performed. 454 Sequencing was carried out according to standard protocols to provide transcriptomes of the target pests. Sequences were assembled and results annotated on the basis of sequence alignment with known published hymenopteran transcriptomes using the Roche software package and annotated using the Blast2Go program, available at http://www.blast2go.org/.

### Example 2

### Identification of Li and Om Target Genes and Sequences

As a general approach to identify target genes, BLAST (NCBI) comparisons using 141 genes identified as being lethal when expressed as RNAi in Drosophila (15, 16) were used to identify 127 orthologous sequences in Nasonia vitiripines (Nv). The identified Nv sequences were further used to screen Om and Li transcriptome libraries, prepared as in Example 1, for lethal genes. Genes that function in the ESCRT pathway were identified in refs. 18, 19 and 20. BLAST (NCBI) comparisons were used to identify orthologous sequences from Nasonia vitripennis. Comparisons using the identified Nasonia vitripennis sequences were further used to screen the Gw 454 transcriptome library for potential target genes. Potential Li and Om target genes were limited to Gw 454 transcriptome sequences that included at least 350 bp in a continuous open reading frame or were at least 50% of the full predicted gene length.

Potential Gw targets were further screened to identify sequences that share limited homology to honey bee, Apis mellifera (Am) sequences. Comparisons were made using a publicly available NCBI B12Seq analysis program (available at http://blast.ncbi.nlm.nih.gov/Blast.cgi?PAGE_TYPE=BlastSearch&PROG_DEF=blas tn&BLAST_PROG_DEF=megaBlast&SHOW_DEFAULTS=on&BLAST_SPEC=bla st2seq&LINK_LOC=align2seq) to identify 100 bp sequences from each Gw target that shared limited (i.e., less than 80%) identity to corresponding Am genes (or, when not possible to identify a 100 bp sequence with less than 80% identity to identify, a shorter fragment of such sequences).

The respective Om target genes and the sequences with limited homology to Am sequences that were identified are set out in SEQ ID NO: 1-24, 47, 48, 51 and 52. Table 1 sets out the SEQ ID NOs for the respective Om target genes and sequences with limited homology identified therein.

TABLE 1
Om Target Sequences and Fragments With Limited Identity
to honey bee (Apis mellifera) sequences

| | | Om Se- |
|---|---|---|
| | | quence <80% |
| | | identical to |
| Dm gene symbol/function | Om Target | Am Sequence |
| (A. mellifera accession no.) | Gene | (% identity) |
| Dmel/ESCRT Pathway | SEQ ID NO: 1 + | SEQ ID NO: 2 |
| (NM_001112672.2) | SEQ ID NO: 70 | (57) |
| Vps23/NADH-ubiquinone | SEQ ID NO: 3 | SEQ ID NO: 4 |
| oxidoreductase, 20 Kd subunit | | (46) |
| (XM_392437.4) | | |
| Vps28/Vacuolar protein sorting 28 | SEQ ID NO: 5 | SEQ ID NO: 6 |
| (XM_392314.4) | | (78) |
| Vps37/mod(r)/ESCRT Pathway | SEQ ID NO: 7 | SEQ ID NO: 8 |
| (XM_001122159.2) | | (54) |
| Vps37b/ESCRT Pathway | SEQ ID NO: 9 | SEQ ID NO: 10 |
| (XM_001120612.2) | | (66) |
| Vps22/lsn | SEQ ID NO: 11 + | SEQ ID NO: 12 |
| Regulation of Notch signaling | SEQ ID NO: 71 | (73) |
| pathway | | |
| (XM_003251158.1) | | |
| Vps25/vesicle-mediated transport | SEQ ID NO: 13 | SEQ ID NO: 14 |
| vacuolar protein sorting 25 | | (69) |
| (XM_395839.4) | | |
| Vps36/vacuolar protein sorting | SEQ ID NO: 15 | SEQ ID NO: 16 |
| (XM_395542.3) | | (66) |
| Vps2/protein transport | SEQ ID NO: 17 | SEQ ID NO: 18 |
| (XM_625161.3) | | (76) |
| Vps20/vacuolar protein sorting 20 | SEQ ID NO: 19 | SEQ ID NO: 20 |
| (XM_394359.3) | | (60) |
| Vps24/Charged multivesicular | SEQ ID NO: 21 | SEQ ID NO: 22 |
| body protein 3 | | (49) |
| (XM_394085.4) | | |
| Snf7/shrub | SEQ ID NO: 23 | SEQ ID NO: 24 |
| ESCRT-III pathway | | (71) |
| (XM_395324.4) | | |
| Mor SWI/SNF complex subunit | SEQ ID NO: 47 | SEQ ID NO: 48 |
| SMARCC2/transcription | | (75) |
| coactivator activity chromatin | | |
| remodeling | | |
| (XM_393008) | | |
| TIF Eukaryotic translation | SEQ ID NO: 51 | SEQ ID NO: 52 |
| initiation factor 3 subunit | | (61) |
| I-like/Eukaryotic translation | | |
| initiation factor 3 subunit | | |
| I-like (XM_392780) | | |

The respective Li target genes and the sequences with limited homology to Am sequences that were identified are set out in SEQ ID NO: 25-46, 49, 50, 53 and 54. Table 2 sets out the SEQ ID NOs for the respective Li target genes and sequences with limited homology identified therein.

TABLE 2
Li Target Sequences and Fragments With Limited Identity
to honey bee (Apis mellifera) sequences

| | | Li Se- |
|---|---|---|
| | | quence <80% |
| | | identical to |
| Dm gene symbol/function | | Am Sequence |
| (A. mellifera accession no.) | Li Target Gene | (% identity) |
| Dmel/ESCRT Pathway | SEQ ID NO: 25 | SEQ ID NO: 26 |
| (NM_001112672.2) | | (53) |
| Vps23/NADH-ubiquinone | SEQ ID NO: 27 | SEQ ID NO: 28 |
| oxidoreductase, 20 Kd subunit | | (46) |
| (XM_392437.4) | | |
| Vps28/Vacuolar protein sorting 28 | SEQ ID NO: 29 | SEQ ID NO: 30 |
| (XM_392314.4) | | (78) |
| Vps37/mod(r)/ESCRT Pathway | SEQ ID NO: 31 + | SEQ ID NO: 32 |
| (XM_001122159.2) | SEQ ID NO: 72 | (67) |
| Vps37b/ESCRT Pathway | SEQ ID NO: 33 | SEQ ID NO: 34 |
| (XM_001120612.2) | | (60) |
| Vps22/lsn | SEQ ID NO: 35 | SEQ ID NO: 36 |
| Regulation of Notch signaling | | (66) |
| pathway | | |
| (XM_003251158.1) | | |
| Vps25/vesicle-mediated transport | SEQ ID NO: 37 + | SEQ ID NO: 38 |
| vacuolar protein sorting 25 | SEQ ID NO: 73 | (68) |
| (XM_395839.4) | | |
| Vps36/vacuolar protein sorting | SEQ ID NO: 39 + | SEQ ID NO: 40 |
| (XM_395542.3) | SEQ ID NO: 74 | (57) |
| Vps2/protein transport | SEQ ID NO: 41 | SEQ ID NO: 42 |
| (XM_625161.3) | | (67) |
| Vps24/Charged multivesicular | SEQ ID NO: 43 | SEQ ID NO: 44 |
| body protein 3 | | (48) |
| (XM_394085.4) | | |
| Snf7/shrub | SEQ ID NO: 45 | SEQ ID NO: 46 |
| ESCRT-III pathway | | (66) |
| (XM_395324.4) | | |
| Mor SWI/SNF complex subunit | SEQ ID NO: 49 | SEQ ID NO: 50 |
| SMARCC2/transcription | | (72) |
| coactivator activity chromatin | | |
| remodeling | | |
| (XM_393008) | | |
| TIF Eukaryotic translation | SEQ ID NO: 53 | SEQ ID NO: 54 |
| initiation factor 3 subunit | | (70) |
| I-like/Translation initiation | | |
| (XM_392780) | | |

### Example 3

### Preparation of dsRNA Triple Gene Silencing Constructs

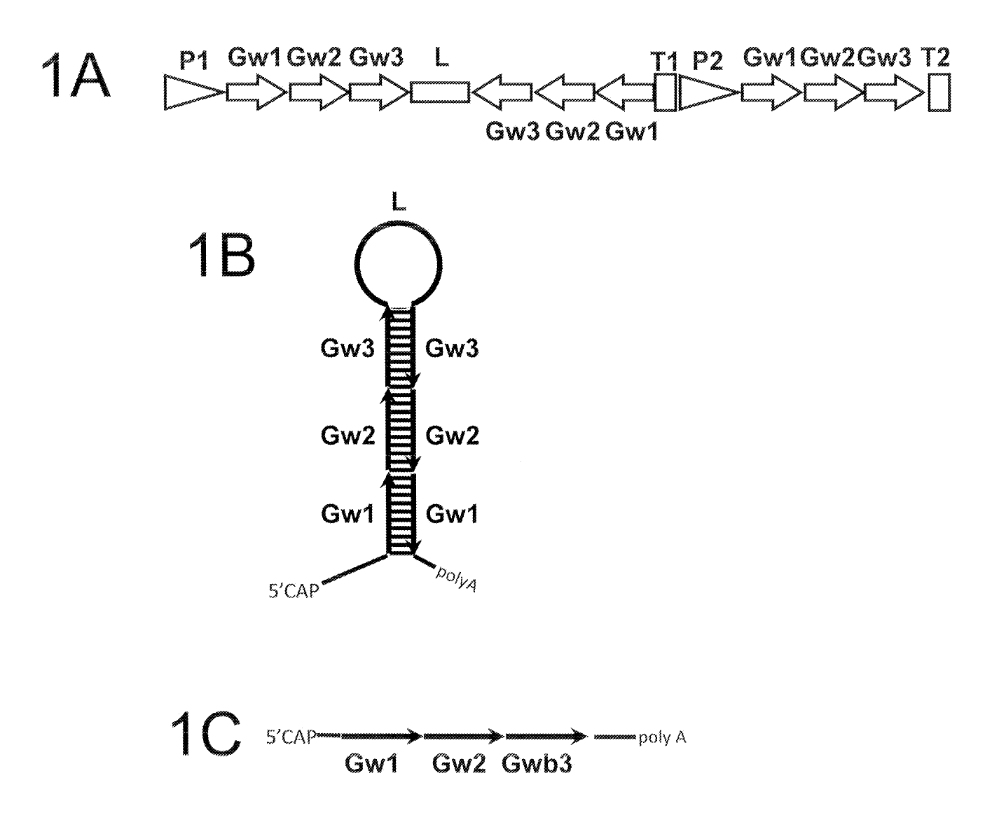

A schematic of the structure of dsRNA triple gene silencing constructs comprising segments from three Gw genes is shown in FIG. 1. Silencing constructs contain two transgenes. A first transgene comprises fragments from each of three Gw genes which are fused and synthesized in inverted repeats, separated by a loop sequence. See FIG. 1A. Transcription of this transgene (initiated at promoter P1 and terminated at T1) produces a hairpin RNA (hpRNA), containing a dsRNA section, formed by annealing of the inverted-repeat sequences of the three Gw genes, and a loop region. See FIG. 1B. A second transgene contains three fused Gw genes, oriented to be transcribed to yield a sense strand with the three gene fragments. See FIGS. 1A and 1C.

The following sequences are used to construct three silencing constructs.

### Om Silencing Construct #1

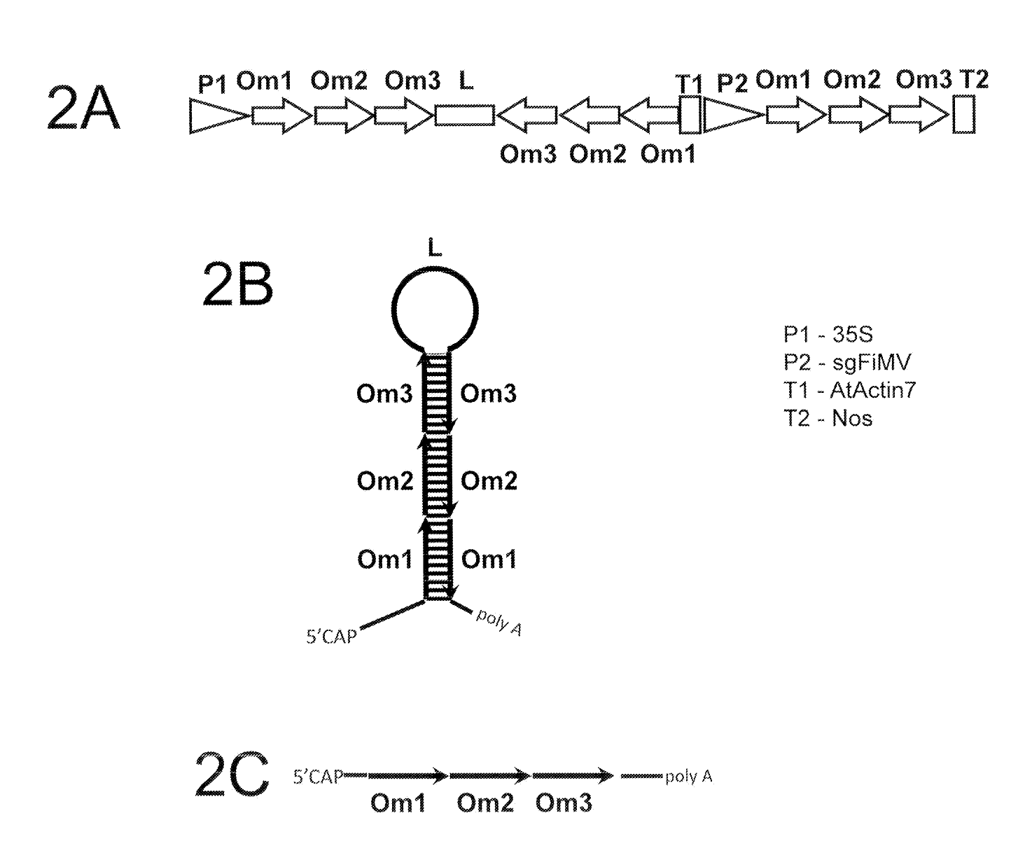

Om Silencing Construct #1 is shown schematically in FIG. 2. Respective 100 bp fragments of each of the Om Snf/shrub gene (SEQ ID NO: 23), Om MOR gene (SEQ ID NO: 47) and, Om TIF gene (SEQ ID NO: 51) are fused and synthesized in inverted repeats separated by 106 bp of a loop sequence (Loop 1; SEQ ID NO: 61). Transcription initiation is driven by the 35S CaMV promoter (SEQ ID NO: 57). Transcription termination is provided by the AtActin7 Terminator (SEQ ID NO: 59). The select 100 bp of SEQ ID NO: 23, 47 and 51 (respectively, SEQ ID NO: 24, SEQ ID NO: 48 and SEQ ID NO: 52) is synthesized in sense orientation between sgFIMV Promoter (SEQ ID NO: 58) to NOS Terminator (SEQ ID NO: 60).

Transcription of construct 1 would yield two mRNAs: (1) A hairpin RNA (hpRNA) with a stem formed by the reverse complementary sequences of the three Om 100 bp sequences, to silence the corresponding Om genes (see FIG. 2B); and (2) sense mRNA of the three, fused Om genes (see FIG. 2C).

The hpRNA formed upon transcription of the hpRNA-forming transgene of Construct #1 has the sequence set out in SEQ ID NO: 55

The respective hpRNA sequences correspond to the following elements:

Nucleotides 1-100 and 607-706: Respective sense and reverse complement sequences of SEQ ID NO: 24, corresponding to nucleotides 1-100 of SEQ ID NO: 23;

Nucleotides 101-200 and 507-606: Respective sense and reverse complement sequences of SEQ ID NO: 48, corresponding to nucleotides 213-312 of SEQ ID NO: 47;

Nucleotides 201-300 and 407-506: Respective sense and reverse complement sequences of SEQ ID NO: 53, corresponding to nucleotides 840-939 of SEQ ID NO: 51; and

Nucleotides 301-406: 106 bp Loop fragment (SEQ ID NO: 61) based on Partial Leptocibe invasa Chitin Synthase intron.

The sense mRNA transcribed from construct 1 comprises the sequence set out in SEQ ID NO: 62)

### Li Silencing Construct #2

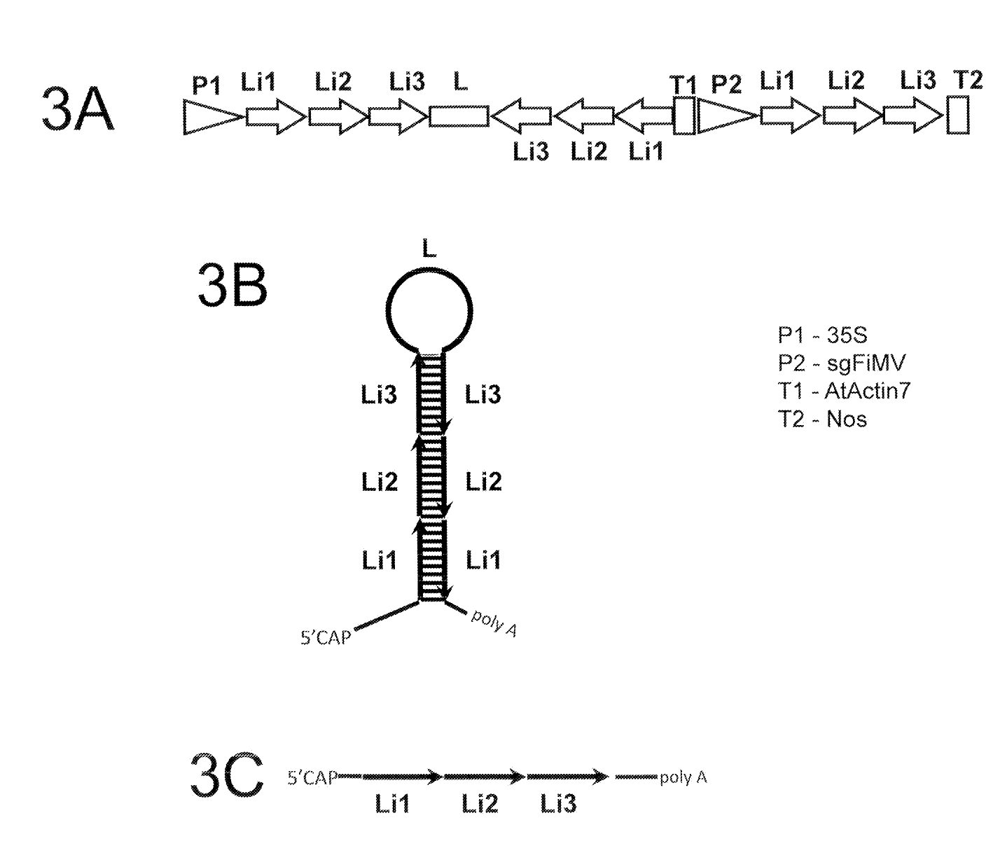

Li Silencing Construct #2 is shown schematically in FIG. 3. Respective 100 bp fragments of each of the Li Snf/shrub gene (SEQ ID NO: 45), Li MOR gene (SEQ ID NO: 49) and, Li TIF gene (SEQ ID NO: 53) is fused and synthesized in inverted repeats separated by 106 bp of a loop sequence (Loop 1; SEQ ID NO: 61). Transcription initiation is driven by the 35S CaMV promoter (SEQ ID NO: 57). Transcription termination is provided by the AtActin7 Terminator (SEQ ID NO: 59). The select 100 bp of SEQ ID NO: 45, 49 and 53 (respectively, SEQ ID NO: 46, SEQ ID NO: 50 and SEQ ID NO: 54) are synthesized in sense orientation between sgFIMV Promoter (SEQ ID NO: 58) to NOS Terminator (SEQ ID NO: 60).

Transcription of construct 2 would yield two mRNAs: (1) A hairpin RNA (hpRNA) with a stem formed by the reverse complementary sequences of the three Li 100 bp sequences, to silence the corresponding Li genes (see FIG. 3B); and (2) sense mRNA of the three, fused Li genes (see FIG. 3C).

The hpRNA formed upon transcription of the hpRNA-forming transgene of Construct #2 has the sequence set out in SEQ ID NO: 56.

The respective hpRNA sequences correspond to the following elements:

Nucleotides 1-100 and 607-706: Respective sense and reverse complement sequences of SEQ ID NO: 46, corresponding to nucleotides 39-138 of SEQ ID NO: 45;

Nucleotides 101-200 and 507-606: Respective sense and reverse complement sequences of SEQ ID NO: 50, corresponding to nucleotides 492-591 of SEQ ID NO: 49;

Nucleotides 201-300 and 407-506: Respective sense and reverse complement sequences of SEQ ID NO: 54, corresponding to nucleotides 840-939 of SEQ ID NO: 53; and

Nucleotides 301-406: 106 bp Loop fragment (SEQ ID NO: 61) based on Partial Leptocibe invasa Chitin Synthase intron.

The sense mRNA transcribed from construct 2 comprises the sequence set out in SEQ ID NO: 63.

### Single-Gene and Double-Gene Control Sequences

Single gene control sequences are generated using a combination of sequences comprising a first sequence of 100 bp sense-100 bp (approximate) loop-100 bp antisense, where “100 bp sense” and “100 bp antisense” refer to complementary sequences from a target gene, and a second 100-bp sense amplifying sequence.

### Example 4

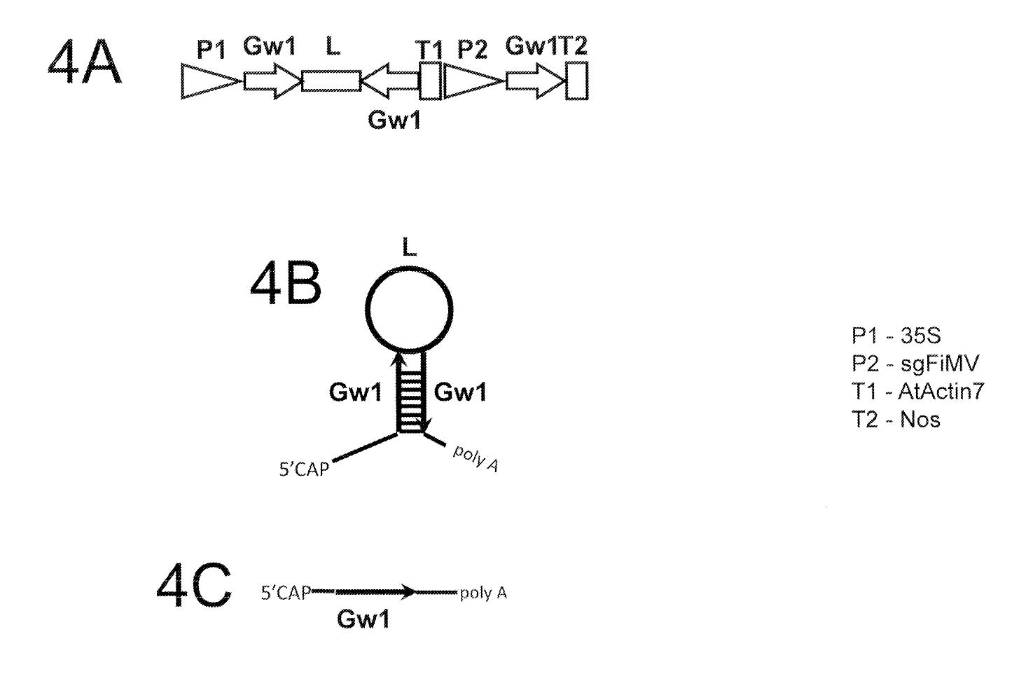

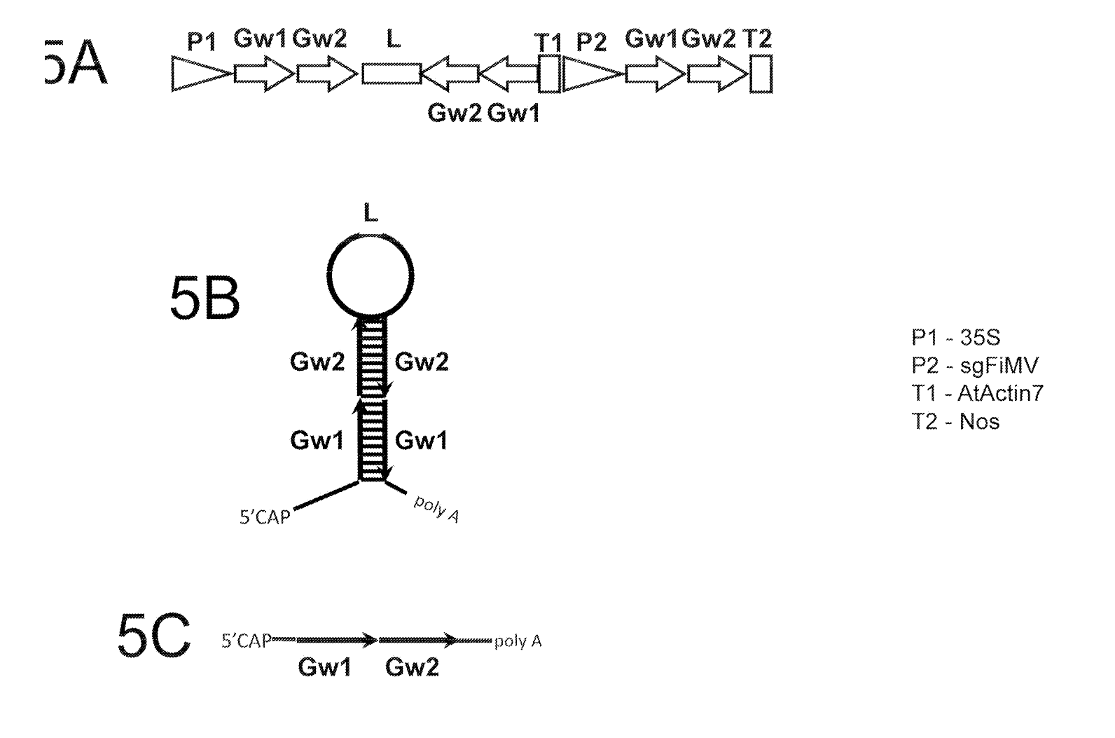

Schematic representations of silencing constructs comprising segments from one and two Gw genes are shown in FIG. 4 and FIG. 5, respectively. Silencing constructs contain two transgenes. A first transgene comprises fragments from each of one (see FIG. 4) or two (FIG. 5) Gw genes which are fused (in the case of constructs containing two Gw genes) and synthesized in inverted repeats, separated by a loop sequence. See FIGS. 4A and 5A. Transcription of this transgene (initiated at promoter P1 and terminated at T1) produces a hairpin RNA, containing a dsRNA section, formed by annealing of the inverted-repeat sequences of the respective Gw genes, and a loop region. See FIGS. 4B and 5B. A second transgene contains the Gw gene(s), oriented to be transcribed to yield a sense strand. See FIGS. 4C and 5C.

### Om Silencing Construct #3 (Om Single Gene Construct)

Single gene control sequences are generated using a combination of sequences comprising a first sequence of 100 bp sense-100 bp (approximate) loop-100 bp antisense, where “100 bp sense” and “100 bp antisense” refer to complementary sequences from a target gene, and a second 100-bp sense amplifying sequence.

To construct silencing Om construct #3, 100 bp fragments the Om Snf 7/shrub gene (SEQ ID NO: 23) are fused and synthesized in inverted repeats separated by 106 bp of a loop sequence (Loop 1; SEQ ID NO: 61). Transcription initiation is driven by the 35S CaMV promoter (SEQ ID NO: 57). Transcription termination is provided by the AtActin7 Terminator (SEQ ID NO: 59). The select 100 bp of Om SEQ ID NO: 23 (i.e., SEQ ID NO: 24) are synthesized in sense orientation between sgFIMV Promoter (SEQ ID NO: 58) to NOS Terminator (SEQ ID NO: 60).

Transcription of construct 3 would yield two mRNAs: (1) A hairpin RNA (hpRNA) with a stem formed by the reverse complementary sequences of the Om 100 bp sequences, to silence the corresponding Om gene (see FIG. 4B); and (2) sense mRNA of the Om gene (see FIG. 4C).

The hpRNA formed upon transcription of the hpRNA-forming transgene of Construct #3 has the sequence set out in SEQ ID NO: 64.

The respective hpRNA sequences correspond to the following elements:

Nucleotides 1-100 and 207-306: Respective sense and reverse complement sequences of SEQ ID NO: 24, corresponding to nucleotides 1-100 of SEQ ID NO: 23;

Nucleotides 101-206: 106 bp Loop fragment (SEQ ID NO: 61) based on Partial Leptocibe invasa Chitin Synthase intron.

The sense mRNA transcribed from construct 3 comprises the Om Snf 7/shrub sequence set out in SEQ ID NO: 24.

### Om Silencing Construct #4 (Om Double Gene Construct)

To construct Om silencing construct #4, 100 bp fragments the Om Snf 7/shrub gene (SEQ ID NO: 23) and Om Vps 28 gene (SEQ ID NO: 5) are fused and synthesized in inverted repeats separated by 106 bp of a loop sequence (Loop 1; SEQ ID NO: 61). Transcription initiation is driven by the 35S CaMV promoter (SEQ ID NO: 57). Transcription termination is provided by the AtActin7 Terminator (SEQ ID NO: 59). The select 100 bp of SEQ ID NO: 23 and 5 (respectively SEQ ID NO: 24 and SEQ ID NO: 6) are synthesized in sense orientation between sgFIMV Promoter (SEQ ID NO: 58) to NOS Terminator (SEQ ID NO: 60).

Transcription of Om construct 4 would yield two mRNAs: (1) A hairpin RNA (hpRNA) with a stem formed by the reverse complementary sequences of the Om 100 bp sequences, to silence the corresponding Om genes (see FIG. 5B); and (2) sense mRNA of the Om gene (see FIG. 5C).

The hpRNA formed upon transcription of the hpRNA-forming transgene of Construct #4 is set out in SEQ ID NO: 65.

The respective hpRNA sequences correspond to the following elements: Nucleotides 1-100 and 407-506: Respective sense and reverse complement sequences of SEQ ID NO: 24, corresponding to nucleotides 1-100 of SEQ ID NO: 23; Nucleotides 101-200 and 307-406: Respective sense and reverse complement sequences of SEQ ID NO: 6, corresponding to nucleotides 350-449 of SEQ ID NO: 5; Nucleotides 201-306: 106 bp Loop fragment (SEQ ID NO: 61) based on Partial Leptocibe invasa Chitin Synthase intron.

The sense mRNA transcribed from construct 5 comprises the sequence set out in SEQ ID NO: 66.

Li Silencing Construct #5 (Li Single Gene Construct) To construct silencing construct 5, 100 bp fragments the Li Snf 7/shrub gene (SEQ ID NO: 45) are fused and synthesized in inverted repeats separated by 106 bp of a loop sequence (Loop 1; SEQ ID NO: 61). Transcription initiation is driven by the 35S CaMV promoter (SEQ ID NO: 57). Transcription termination is provided by the AtActin7 Terminator (SEQ ID NO: 59). The select 100 bp of Li SEQ ID NO: 45 (i.e., SEQ ID NO: 46) is synthesized in sense orientation between sgFIMV Promoter (SEQ ID NO: 58) to NOS Terminator (SEQ ID NO: 60).

Transcription of construct 3 would yield two mRNAs: (1) A hairpin RNA (hpRNA) with a stem formed by the reverse complementary sequences of the Li 100 bp sequences, to silence the corresponding Li gene (see FIG. 4B); and (2) sense mRNA of the Li gene (see FIG. 4C).

The hpRNA formed upon transcription of the hpRNA-forming transgene of Construct #5 has the sequence set out in SEQ ID NO: 67.

The respective hpRNA sequences correspond to the following elements: Nucleotides 1-100 and 207-306: Respective sense and reverse complement sequences of SEQ ID NO: 46, corresponding to nucleotides 39-138- of SEQ ID NO: 45; Nucleotides 101-206: 106 bp Loop fragment (SEQ ID NO: 61) based on Partial Leptocibe invasa Chitin Synthase intron.

The sense mRNA transcribed from construct 3 comprises the L9 Snf 7/shrub sequence set out in SEQ ID NO: 45.

### Li Silencing Construct #6 (Li Double Gene Construct)

To construct Li silencing construct #6, 100 bp fragments the Li Snf 7/shrub gene (SEQ ID NO: 45) and Li Vps 28 gene (SEQ ID NO: 29) are fused and synthesized in inverted repeats separated by 106 bp of a loop sequence (Loop 1; SEQ ID NO: 61).

Transcription initiation is driven by the 35S CaMV promoter (SEQ ID NO: 57). Transcription termination was provided by the AtActin7 Terminator (SEQ ID NO: 59). The select 100 bp of SEQ ID NO: 45 and 29 (respectively SEQ ID NO: 46 and SEQ ID NO: 30) are synthesized in sense orientation between sgFIMV Promoter (SEQ ID NO: 58) to NOS Terminator (SEQ ID NO: 60).

Transcription of Li construct 6 would yield two mRNAs: (1) A hairpin RNA (hpRNA) with a stem formed by the reverse complementary sequences of the Li 100 bp sequences, to silence the corresponding Li genes (see FIG. 5B); and (2) sense mRNA of the Li sense (see FIG. 5C).

The hpRNA formed upon transcription of the hpRNA-forming transgene of Construct #6 is set out in SEQ ID NO: 68.

The respective hpRNA sequences correspond to the following elements:

Nucleotides 1-100 and 407-506: Respective sense and reverse complement sequences of SEQ ID NO: 46, corresponding to nucleotides 39-138 of SEQ ID NO: 45;

Nucleotides 101-200 and 307-406: Respective sense and reverse complement sequences of SEQ ID NO: 30, corresponding to nucleotides 412-511 of SEQ ID NO: 29;

Nucleotides 201-306: 106 bp Loop fragment (SEQ ID NO: 61) based on Partial Leptocibe invasa Chitin Synthase intron.

The sense mRNA transcribed from construct 6 comprises the sequence set out in SEQ ID NO: 66.

### Example 5

### Expression of RNAi Constructs in Eucalyptus

dsRNA constructs #1 and #2 are transformed into eucalyptus using a protocol essentially described in Prakash et al., In Vitro Cell Dev Biol.-Plant, 2009, 45:429-434. Briefly, shoots of Eucalyptus are propagated in vitro on Murashige and Skoog (MS) basal salt medium consisting of 3% (w/v) sucrose and 0.8% (w/v) agar. All in vitro plant materials are incubated at 25±2° C. under a 16-h photoperiod with cool white fluorescent lamps with an intensity of 30 μEm⁻² s⁻¹. A. tumefaciens strain LBA 4404 harboring a binary vector pBI121 containing nptII gene is used for transformation. Bacterial culture collected at late log phase are pelleted and resuspended in MS basal salt medium. Leaves from in vitro material are collected and used as explants for transformation experiments.

Explants are precultured on the MS regeneration medium supplemented with 0.5 mg/1 BAP and 0.1 mg/l NAA for 2 d. Precultured leaf explants are gently shaken in the bacterial suspension for 10 min and blotted dry on a sterile filter paper. Explants are then cocultivated in medium under the preculture conditions for 2 d. Following cocultivation, explants are washed in MS liquid medium, blotted dry on a sterile filter paper, and transferred to MS regeneration medium containing 0.5 mg/1 BAP and 0.1 mg/l NAA supplemented with 40 mg/1 kanamycin and 300 mg/1 cefotaxime. After 4-5 weeks of culture, regeneration is observed and explants are transferred to liquid elongation medium (MS medium supplemented with 0.5 mg/1 BAP, 40 mg/1 kanamycin, and 300 mg/1 cefotaxime) on paper bridges. The elongated shoots (1.5-2 cm) are propagated on MS medium with 0.1 mg/1 BAP. Leaf segments from regenerated and elongated shoots are analyzed by PCR and western blot. Positive shoots are multiplied to 10 copies on MS medium containing 0.04 mg/L BAP. A few leaves are excised from the shoots and analyzed by RT-PCR.

Expression of dsRNAs is measured using RT-PCR. Total RNA from 50 mg fresh transgenic plant tissue is purified using EPICENTRE MasterPure™ Plant RNA Purification Kit (Cat. #MPRO9010) followed by DNAse treatment with Ambion TURBO DNA-Free™ Dnase (Cat. #AM1907). 1 μl of total RNA from each sample is analyzed by RT PCR. RT PCR is performed using Invitrogen SuperScript III One-Step RT-PCR System with Platinum Taq DNA Polymerase kit (Cat. #12574-018). As a control, the Platinum Taq DNA Polymerase kit (Cat. #12574-018 and #10966-018) is used to recognize traces of DNA contaminations. No fragment amplification is expected for this control.

### Example 6

### Bioassay of Li and Om dsRNA Constructs

To prepare a gall tissue homogenate, galls are opened and all larvae are removed. Larvae-free galls and surrounding leaf area are then homogenized in liquid nitrogen with mortar and pestle until a fine powder homogenate is achieved. An agar-based wasp larvae artificial feed is prepared by dissolving agar (50 mg) at 100° C. in buffer, cooling to 45° C. and adding 5 g of gall tissue homogenate and bringing total volume to 10 ml. Aliquots of artificial feed (10 μl) are placed in a sealable tube and allowed to cool to room temperature. Gall wasp larva are isolated from galls with live larvae from which adult gall wasps have not yet emerged, by cutting gall lids from plant tissue surface to expose the gall interior and collecting larvae using a sharp-tipped rod by gently contacting the larvae. Individual larvae are placed in each tube of artificial feed. Tubes are humidified by placing a drop of water in each tube, and tubes are sealed and incubated at 25° C.

Artificial feed is prepared from gall tissue prepared from eucalyptus plants that are transformed with silencing constructs and control plants (i.e., wt untransformed plants or plants transformed with vector alone, without insertion of Li or Om nucleic acids or without nucleic acids that could form siRNAs). Effects of Li and Om dsRNA are determined by examining effects on development of gall wasp larvae grown on artificial feed prepared from transformed eucalyptus tissue, compared to larvae grown on artificial feed prepared from wt and control transformed eucalyptus tissue.

### Example 7

### Test of Protective Effect of Li and Om dsRNA Constructs

Eucalyptus plants are transformed with the Li and Om silencing constructs described herein and transgenic lines are established. Controls lines are established by transforming plants with vector alone, without insertion of Li or Om nucleic or without nucleic acids that could form siRNAs.

Transgenic, wt, and control eucalyptus plants are grown in insect proof cages in the greenhouse together with adult gall wasps. The insect proof cages keep the inoculums in while preventing outside pests from entering the cage. Following wasp inoculation, the appearance of galls in the veins and in the leaves is evaluated. Plants are examined to determine number of galls, gall size (maximum length), number of vital larvae in galls and the number of emerging matured gall wasps. Five independent transformation events of transgenic eucalyptus plants transcribing dsRNA are tested. Ten lines of each transformation event are inoculated with adult gall wasps in 3 independent repeats. Number of galls, gall size, vital larvae per 10 galls and emerging adults (by the exit hole) are recorded 1, 2, 3 and 4 months after inoculation.

Exemplary prophetic result: Transgenic plants transcribing dsRNA targeting gall wasp genes exhibit fewer galls of smaller size, compared to controls and galls do not develop further. No vital larvae are detected in the small galls and no adult wasps emerge. Transgenic plants lines are resistant to gall wasp infection, compared to control and wt plants that are infected with fully developed galls.

A number of embodiments of the invention have been described. Nevertheless, it will be understood that various modifications may be made without departing from the spirit and scope of the invention. Accordingly, other embodiments are within the scope of the following claims.

All patents, patent publications and non-patent literature referenced in the specification are hereby incorporated herein by reference in their entireties.

### REFERENCES

1. Baum J A, et al., Control of coleopteran insect pests through RNA interference, (2007), Nat Biotechnol. 25:1322-6.
2. Frizzi A, et al., Tapping RNA silencing pathways for plant biotechnology, (2010), Plant Biotechnol 8:655-77.
3. Gordon K H, et al., RNAi for insect-proof plants, (2007), Nat Biotechnol 25:1231-2.
4. Huvenne H, et al., Mechanisms of dsRNA uptake in insects and potential of RNAi for pest control: a review, (2010), J Insect Physiol 56:227-35.
5. Mao Y B, et al., Silencing a cotton bollworm P450 monooxygenase gene by plant-mediated RNAi impairs larval tolerance of gossypol, (2007), Nat Biotechnol 25:1307-13.
6. Mendel Z, et al., The taxonomy and natural history of Leptocybe invasa (Hymenoptera: Eulophidae) gen & sp. nov., an invasive gall inducer on Eucalyptus, (2004), Australian J Entomol, 43:101-13.
7. Nunes F M, et al., A non-invasive method for silencing gene transcription in honeybees maintained under natural conditions., (2009), Insect Biochem Mol Biol 39:157-60.
8. Price D R, et al., RNAi-mediated crop protection against insects, (2008), Trends Biotechnol 26:393-400.
9. Protasov A, et al., Biology, revised taxonomy and impact on host plants of Ophelimus maskelli, an invasive gall inducer on Eucalyptus spp. In the Mediterranean area, (2007), Phytoparasitica 35:50-76.
10. Tinoco M L, et al., In vivo trans-specific gene silencing in fungal cells by in planta expression of a double-stranded RNA, (2010), BMC Biol 8:27.
11. Hannon, G. J., RNA interference, (2002), Nature 418:244-251
12. Baulcombe, D., RNA silencing in plants, (2004), Nature 431:356-363.
13. Pei Y, et al., On the art of identifying effective and specific siRNAs, (2006), Nature Methods 3(9):670-676.

14. Cullen, B R., Enhancing and confirming the specificity of RNAi experiments, (2006), Nature Methods 3(9):677-681.

15. Chen et. al, New Genes in Drosophila Quickly become essential, Science (2010), 330:1682-5.
16. Dietzl et. al., A Genome Wide Transgenic RNAi Library for Conditional Gene Inactivation in Drosophila. Nature (2007), 448:151-7.
17. Baum et al., Control of Coleoptern Insects Pests Through RNA Interference. Nature Biotechnology (2007) 25: 1322-26.
18. Vaccari et al., Comparative Analysis of ESCRT-I, ESCRT-II and ESCRT-III Function in Drosophila by Efficient Isolation of ESCRT Mutants. Journal of Cell Science (2009) 122: 2413-23.
19. Djeddi et al., Induction of Autophagy in ESCRT Mutants is an Adaptive Response for Cell Survival in C. Elegans. Journal of Cell Science. (2011) 125: 685-694.
20. Michelet et al. Developmental and Cellular Functions of the ESCRT Machinery in Pluricelular Organisms. Biol. Cell (2010) 102, 191-202.

SEQUENCES
SEQ ID NO: 1 + SEQ ID NO: 70
Organism: Om
Dmel
ESCRT Pathway (P)
A mellifera NM_001112672.2
SEQ ID NO: 1
ATGTCTAAGAAGGATAGCAACAAAATGCTGGTTCACCAGTTATCATCCGT
ATTACCAGACGATCGACCAATCACAGCAATAAATATTATCGAGGATTTGG
ATAAGTGTCCACCTAATTTCACTGTTGTATCAAGGACCTATGACTTAGAT
TCAGATGCAGACTTATGGCGAGAAAGTGGACTATTTATCAAAAAGAAATC
CAGATACATATGTTTC [gap]
SEQ ID NO: 70
ATCGATGAGAGAGAACCTCCTCCAGAAGGATTTAGTATGATCTCTCACAC
TGTAGATACAATGCAAAAAGCGTGGCGTAAAAGACAACTGTGTTATAAAA
TTAGAACTAGAGACTTATGCCCATGCGCAGTATCGGATATAATTATTTGC
AATAGGATTGGTAAAGGCTCAAAAATGGCACCAAATGAATTTACTTATGC
TGGTGTCATCAATGGAGTTTATATTTGTTACAAAACTGTACCCCTCTCTG
CAAGCCCGTTATACTCTGCACAGTCATATGCTAATATAGAATTATTCCAA
ACGTTGTCTCCAACTCTGTCAAACGGTACACCGCACAGAGTGGCACCAGA
GAGGCCACCAAAGCCAAAATTCACGCCGAAACCTACAAATGGACTGTATC
CGCAAATAAGTCCAGTAAACGCAGGAAAGACTGCAGCGGATGAGAATGAC
CAAGACTATGAAGTACTAAGTCCAAGTGCCAGAATAAGGCCAACGCGACC
AGCGCCAAGGCCACCGTCTAATAAATCGCCATTGGTTCATTCTGTGCCAG
CTTATGGAACATTGACAGGTTCATCAGATCTCGACGGAGTTCCTTTTGTT
CTCAATCCTCTTTTGAGCATGAGTACATCAATTATGTTAAATAAACAGTT
TCCTGTGATTAAAATCCGAACTCAGAACGAGCTGGATAAAGAGTATTTCT
ACGACTTTCGAACTGAGAGAGAAACTTGA
SEQ ID NO: 2
Organism: Om
SEQ ID NO: 1 nucleotides 682-781 (57)
GTACTAAGTCCAAGTGCCAGAATAAGGCCAACGCGACCAGCGCCAAGGCC
ACCGTCTAATAAATCGCCATTGGTTCATTCTGTGCCAGCTTATGGAACAT
SEQ ID NO: 3
Organism: Om
Vps23
NADH-ubiquinone oxidoreductase, 20 Kd
subunit (F)
A. mellifera XM_392437.4
ATGTTGCGTTCCCTTTTAGCAAACCATACCTTTCAACAAGGTCTCAATTT
GGTGGCACGTAACAATGCTGCTATCTTGCGATGTCAGAATCCACTCTTGG
AGCATGCTAAAACAATGGCAACACAATCGGTAAATCAAAGTTCTGGAAGT
TCAGCTCCAGCACCAGCAAAAAAAGAACCTTACAGTCCATTCCAAAACAC
AAGTAACATGGTTGAATATGCCTTAGCTAGACTAGACGATCTGTTAAATT
GGGGAAGAAAAGGTTCAATGTGGCCACTGACGTTCGGGCTGGCATGTTGT
GCTGTGGAAATGATGCACATTGCTGCTCCACGATACGATATGGACAGATT
TGGTGTAGTTTTCCGTGCTTCTCCTAGGCATGCTGATGTTATCATAGTAG
CTGGTACAGTGACAAATAAAATGGCACCGGCGTTGAGAAAAGTATACGAT
CAGATGCCTGATCCCAAATGGGTCATCTCAATGGGTAGTTGTGCAAATGG
TGGAGGCTATTATCACTACAGTTACTCAGTTGTAAGGGGTTGCGATAGAA
TTATACCTGTGGACATTTATGTACCTGGCTGTCCTCCAACAGCTGAAGCA
TTACTTTATGGAGTTTTACAGCTGCAGAAAAAGGTGAAACGTATGGAGAC
TGCCCAAGTGTGGTACAGAAAATAG
SEQ ID NO: 4
Organism: Om
SEQ ID NO: 3 nucleotides 010-109 (46)
TCCCTTTTAGCAAACCATACCTTTCAACAAGGTCTCAATTTGGTGGCACG
TAACAATGCTGCTATCTTGCGATGTCAGAATCCACTCTTGGAGCATGCTA
SEQ ID NO: 5
Organism: Om
Vps28 Vacuolar protein sorting 28 (F)
A. mellifera XM_392314.4
ATGTCTATTGCACAAGATCGTCCTGAACTTTTTGAAGAAGTAAAGCTGTA
CAAAAATGCTAGGGAACGTGAAAAATATGATAACCAGGCCGATTTATATG
CTGTTGTTAATACTTTACAGCATTTAGAGAAAGCTTATATCAGAGACTGT
GTTACGCCCAAAGAATATACAGCTGCATGCAGCAAATTATTAGTTCAATA
TAGAGCTGCTTTTAAACAGGTCCAGAGCGATCAATTTCCAACAATTGATG
CTTTTGCCAGAGCATTTAGACTTGATTGTCCAGCAGCACTTGAACGAATT
AAAGAAGATAGACCTATCACCATTAAAGATGACAAAGGCAATACTTCTAA
ATGTATTGCTGACATTGTTTCACTCTTCATAACATTAATGGATAAGTTAC
GACTTGAAATTAAAGCTATGGATCAACTTCATCCAGATCTTAGAGATTTG
ATGGACACTATGAACAGGCTTAGTATATTGCCAAGCGATTTTGATGGAAA
GCAAAAAGTTGCAGAATGGCTTCAGACCCTAAATAATATGTCAGCATCTG
ATGAACTATCGGATACTCAAGTTAGACAACTGATATTCGATTTAGAAACA
TCTTACAATGCTTTTAATAGAGTTTTACATAACTCGTAA
SEQ ID NO: 6
Organism: Om
SEQ ID NO: 5 nucleotides 350:449 (78)
AATGTATTGCTGACATTGTTTCACTCTTCATAACATTAATGGATAAGTTA
CGACTTGAAATTAAAGCTATGGATCAACTTCATCCAGATCTTAGAGATTT
SEQ ID NO: 7
Organism: Om
Vps37/mod(r)
ESCRT Pathway (P)
A. mellifera XM_001122159.2
ATGTTATCGCGAATATTCCTTGGGGAAAATGAAAATGCCGCAGTCAAGAG
AAAACGTCAGATCGACACGCTGAAAATTTTCAACGACAATGTTCTGGAGC
TGCAAGAAGATGTCGATTATCAAGTGCACTTTGATGCAGGAGGCAAAAGA
ATGGCTATTCAGGTCTCGCTATCTCCAGACTTTCCACTGGAAAAGCCAGT
ACTTAGAGTATCACCTCCGATAAAACATAAATGGTGCAATGAACACAGTG
AAATCACTAGTGCTCCAGGATTATTAAATTTTACAGTACACAGTGATCTG
GGTCGTGTTGTTCAAGCCATTATACGAGAATTTAGTAAAAATCCACCACA
GTTGTTAGAAGAAAGTTCTCCAGTATCAAATCTAGCGCTTGGAGATTTAG
CTGGAAGAACGTCACCATCTTCTTACATGTGGAAACCATGTGATCTTGCC
TCAGGATCTTATAACTCATACTACAATCCTTCATTCCAGCAGTTTTCTTC
GTCAAATGCTACACCTAGCATTTATAACTATAATTACACAAATGCTAGCA
ATACATATTTGGGCAATTCGCTAAGCTCCGTTCAGTTTAATACAACAAAT
GCTCAATCACAGTACTCATCGAGTCCTAGCGCAAATGCTTACACAAATTT
ACATTATGCTAATGCCAACATGGGACCATCGATGCAGAATCAAGTGCAAT
ATAAAAGTAATAAAGTAACC
SEQ ID NO: 8
Organism: Om
SEQ ID NO: 7 nucleotides 361-460 (54)
GAAAGTTCTCCAGTATCAAATCTAGCGCTTGGAGATTTAGCTGGAAGAAC
GTCACCATCTTCTTACATGTGGAAACCATGTGATCTTGCCTCAGGATCTT
SEQ ID NO: 9
Organism: Om
Vps37b
ESCRT Pathway (P)
A. mellifera XM_001120612.2
ATGTTTAAACCACCACAAGAACCAAATATAGCTGCTGCTATTGCACCAAT
TTCAGATTTAAGTACGGATGCATTGAAAGAGTTGTTGAATGATGATGAAA
AGTTTGAAGAGATTATCAAAGATAATCAACAGTTACTAGAGTTAGAATCA
GAAAAAGAAGAGATTATGGTACGGAATCGATCGCTGGCAGAATTTAATTT
ATCAAAAGAACCAGAATTGGAGGAATGTAAAAGGAAAATACAATCACTTA
GCGAAGAAGGCAATAATCTTTGCGCTAGTGTGCAAGCCAAACTTGAACAA
ATAACAAATAAAGCTGGAACTATGACAGTTGATACT
SEQ ID NO: 10
Organism: Om
SEQ ID NO: 9 nucleotides 95-194 (66)
ATGAAAAGTTTGAAGAGATTATCAAAGATAATCAACAGTTACTAGAGTTA
GAATCAGAAAAAGAAGAGATTATGGTACGGAATCGATCGCTGGCAGAATT
SEQ ID NO: 11 + SEQ ID NO: 71
Organism: Om
Vps22/lsn
Regulation of Notch signaling pathway (P)
A. mellifera XM_003251158.1
SEQ ID NO: 11
ATGAGGAGAAAACCTGGAGTTGGAGCAATTCATAAACAAAAGTATGAACA
AGAAAAATATAAAGATAAAGGAACGGAGCTTCAAGAAAATCAATTCGAGC
AGATGACGAAGCAACTGGAAACTTTTCGAATAAATTTAGAAGACTTTGCT
TCAAAACACAAGAATGAAATAAAGAAAAATGCGCACTTCAGGAAGCAATT
TACAGATATGTGTGCCTCAATTGGTGTAGATCCATTAGCATCAGGAAAAG
GATTTTGGTCTGTTCTTGGTATAGGAGACTTTTATTATGAAATTGCAGTA
CAAATTGTAGAAGTTTGCTTGGCGACAAACTATAAAAATGGAGGTTTGAT
ATCTTTAGACGAATTA [gap]
SEQ ID NO: 71
CAAAAAGCTTTAGATCATATGGTTAAAGAAGGATTAGCTTGGATAGATGA
ACAAAATGAAGAACCCTTGTACTGGTTTCCAAGTCTTTTTACAGCTTGCA
TTGCTTCAAAAACATAG
SEQ ID NO: 12
Organism: Om
SEQ ID NO: 11 nucleotides 110-209 (73)
AGCAACTGGAAACTTTTCGAATAAATTTAGAAGACTTTGCTTCAAAACAC
AAGAATGAAATAAAGAAAAATGCGCACTTCAGGAAGCAATTTACAGATAT
SEQ ID NO: 13
Organism: Om
Vps25
vesicle-mediated transport vacuolar
protein sorting 25 (P)
A. mellifera XM_395839.4
ATGACTGAAATTGACTGGCCCTGGCAATATAGTTTCCACCTTTTTTTCAC
ACTTCAACCCCATACAGAAACAAGAGTAAAGCAGATAGAGGCATGGAAAA
CTTTAGTTTTGAATTACTTCAGGATAGCTAAACAAGCGATTCTTGATGTT
CGAGAAATCCATAGCACTCCATTGTTTAACAACACTTCTATTGACAGAAA
GCTACCACTAGAAGTCGTATCTATAATAGTAGAAGAATTAGCAAAATCAG
GTAATGCAATACCTTTGGATAAATTGAAACAACGATGGATTATATCGTGG
CACACATTAGAAGAATGGGCTGATACTATATACTCGTGGGCACAAGCAAA
TGGATTTATTGGGTCTGTTTGTACACTTTATGAACTAACACAAGGAGAGA
ATACAGTGGATGAAGAGTTTTATGGTTTGGACAACGAATTACTTATAAGA
TCTCTAAGGACACTGGAAGCTTCTAAAAAGGCAGAACTAATTATGTTTGA
C
SEQ ID NO: 14
Organism: Om
SEQ ID NO: 13 nucleotides 140-239 (69)
TTCTTGATGTTCGAGAAATCCATAGCACTCCATTGTTTAACAACACTTCT
ATTGACAGAAAGCTACCACTAGAAGTCGTATCTATAATAGTAGAAGAATT
SEQ ID NO: 15
Organism: Om
Vps36
vacuolar protein sorting (P)
A. mellifera XM_395542.3
TTTAAATCTTATCTCATGAGTCTTGGTATTGATGATCCAGTTACTAGAGA
GGCCTATAAAAGTAGTAACGAATATTTCAAACAACTTGCCAGGCAGTTAG
CTGAAATTTTGGATGAACCAATTAGGGAGGTAGGTGGAATGATGGCTTTA
ACTGATGTATACTGTCGCGTCAATAGAGCAAGAGGTTTGGAGCTTCTCTC
ACCTGAAGATTTACTTCATGCGTGTAGACAATTAGCTCCATTAAATCTGC
CAATAGTATTAAGGATCTTTGACAGCGGTGTTATGGTTCTACAATCTAGA
GCACATAACGATTATGAAATTGCTGAAGCTGTAGCTCAATTGATCAAGAG
CAGAGGCTCGATAACAGCAACGGAACTGGCACAATCTGAAGGTATATCAA
TGGTTTTAGCTTGCGAAAGGCTTCTAATGACTGAAAAATAC
SEQ ID NO: 16
Organism: Om
SEQ ID NO: 15 nucleotides 293-392 (66)
AATCTAGAGCACATAACGATTATGAAATTGCTGAAGCTGTAGCTCAATTG
ATCAAGAGCAGAGGCTCGATAACAGCAACGGAACTGGCACAATCTGAAGG
SEQ ID NO: 17
Organism: Om
Vps2
protein transport (P)
A. mellifera XM_625161.3
ATGGCTAAAGATGGACAAATGGATGCGGTTAAAATTATGGCAAAAGATCT
TGTGAGGACTAGGCGCTATGTTAAGAAATTCATGTTAATGAAAGCGAATA
TTCAAGCTGTGTCCCTCAAAATACAAACTCTTAAATCTCAAAACACAATG
GCTCAAGCAATGAAAGGTGTCACAAAAGCTATGCAAAATATGAACAAGCA
ACTCAACTTACCTCAAATCCAAAAGATTTTGCAAGAGTTTGAGAAACAAT
CCGAAGTCATGGACATGAAAGAAGAAATAATGAACGATGCGATTGATGAT
GCAATGGAAGATGAGGGTGATGAAGAGGAAAGTGACGCAATTGTATCCCA
AGTTCTGGACGAACTAGGCCTCCAACTCACCGACCAATTGTCCGGTCTAC
CTCAAGCCTCAGGGTCTCTGAGTATAGCAGGCTCCAAGCAACCAGTA
SEQ ID NO: 18
Organism: Om
SEQ ID NO: 17 nucleotides 332-431 (76)
GTGACGCAATTGTATCCCAAGTTCTGGACGAACTAGGCCTCCAACTCACC
GACCAATTGTCCGGTCTACCTCAAGCCTCAGGGTCTCTGAGTATAGCAGG
SEQ ID NO: 19
Organism: Om
Vps20 vacuolar protein sorting 20 (P)
A. mellifera XM_394359.3
TTAGAGAATTTAGAGCGAATGGTACATGACTTGGAGTTTGCACAAGTTGA
AGCTAAAGTTATCGATGGCCTTAAAGTCGGCAATGATGCCCTCAAAAAGT
TACATGCAATATTATCAATTGATGAAATTGAAAAAGTCATGGATGAAACC
AGAGAAGGTGTAGAGAAACAACAAGAAATTGATGCACTTTTGTCTGGAGC
ACTGAGTGATGAAGATGAAAATGATGTTGAAACTGAACTTAATGCTCTTA
TCTCGCAAGAAGCCGATGTAATAACCACGCCTGAAGTACCAGAAGATGTT
CCAATGCCTGAC
SEQ ID NO: 20
Organism: Om
SEQ ID NO: 19 nucleotides 213-312 (60)
AGATGAAAATGATGTTGAAACTGAACTTAATGCTCTTATCTCGCAAGAAG
CCGATGTAATAACCACGCCTGAAGTACCAGAAGATGTTCCAATGCCTGAC
SEQ ID NO: 21
Organism: Om
Vps24
Charged multivesicular body protein 3 (P)
A. mellifera XM_394085.4
ATCCAGAGAGAAGAAGAAAAAGTGAAAAGATCATTAAAAGATGCAGCCAA
AAAAGGTGATAAAGATGTATGTAAAGTATTGGCAAAAGAAGTTATTCGAG
CCAGAACTGCATGTAATAAACTGCATACATCGAAAGCACATCTTAATTCA
GTTACCTTACAAATGAAAAATCAGCTGGCAACAATAAGAGTAGCTGGATC
TCTTTCAAAATCAACAGAAGTTATGCAAGCAATGCAAGCTCTCGTAAAAG
TTCCAGAAGTTGCAGCAACCATGAGAGAACTTTCAAAAGAAATGATGAAA
GCTGGGATAATTGAAGAAATGATGGATGAAACCTTGGATTCTATTGAAGA
TTCTGAAGAATTGGAAGATGCAGCAGATGAAGAAGTTGATAAAATTTTAT
GGGAAGTCACTGCTGGACAGATGGGAAGAGCGCCTGATGTTGTCACTGAT
ACACCTGGAGCATCTACGTCTAAAGAAGAAGAAGTTGAAGAAGTCACGGA
TGACAAAGAATTAGAAGAAATGAAAAATAGGTTACAAAGCCTTCGCAGCT
AG
SEQ ID NO: 22
Organism: Om
SEQ ID NO: 21 nucleotides 015-114 (49)
AGAAAAAGTGAAAAGATCATTAAAAGATGCAGCCAAAAAAGGTGATAAAG
ATGTATGTAAAGTATTGGCAAAAGAAGTTATTCGAGCCAGAACTGCATGT
SEQ ID NO: 23
Organism: Om
Snf7/shrub
ESCRT-III pathway (F)
A. mellifera XM_395324.4
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
TCAAGAAGCAAGACTTCCTCGAGGGCAAGATCGAGCAGGAAATTCAGCAG
GCTAGGAAATACGGAACTAAAAACAAACGAGCTGCTATCCAAGCATTAAA
AAAGAAGAAACGCTATGAGAAACAACTGCAACAAATCGATGGCACATTAT
CCACAATTGAGATGCAGAGAGAGGCACTCGAAAGCGCCAACACGAATACT
GCTGTTCTGACTACGATGAAGAATGCAGCAGATGCTTTGAAAGCTGCTCA
TCAACACATGGATGTTGATCAAGTCCATGATATGATGGATGACATTGCTG
AACAACAAGATGTGGCAAAGGAAATTTCAGATGCTATCTCTAATCCAGTA
GCATTCGGCCAAGACATAGATGAAGATGAACTTGAGAAGGAACTAGAGGA
ATTAGAACAAGAAGAATTAGATAAAGAATTGTTGGGTATAAAGACTACAG
ACGAATTACCGTCAGTTCCTGCTACATCATTGCCAGCTGTACCAGAAAAG
AAAACAAGAGCAAAAGCAGAAGATGATGATGATTTGAGAGAACTGGAACA
ATGGGCATCGTAA
SEQ ID NO: 24
Organism: Om
SEQ ID NO: 23 nucleotides 001-100 (71)
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
Li Sequences
SEQUENCE LISTING
SEQ ID NO: 25
Organism: Li
Dmel
ESCRT Pathway (F)
A mellifera NM_001112672.2
ATGTCGAAGAAGGATGGTAACAAAATGCTGGTTCACCAGTTATCGTCTGT
TCTACCGGATGATAGGCCTATCACAGCAATAAATATCATAGAAGACTTGG
ACAAGTGTCCACCAAACTTCACTGTGGTATCAAGAACATATGATTTGGAT
TCAGATGCAGACTTGTGGCGAGAGAGTGGGCTGTTTATTAAGAAAAAGTC
AAGATACATATGTTTTTCAAAGACTGAAGGATTGCCACATTCTGTTATAG
AAAAAATTTCAATTCTTGATGAGAGAGAACCACCTCCAGAGGGATTCAGT
ATGATTTCTCATACTGTAGACTCAATGCAAAAAGCATGGCGTAAAAGGCA
ACTGTGTTACAAAATTAGAAATAAGGACTTGTGCACATGTGCAGTATCGG
ACATAATTATCTGTAGTAGGCTTGGTAAAGGTTCCAAACTGGCTCCAGAT
GAATTTACCTACGCTGGTGTCATAAACGGTGTTTACCTTTGCTATAAAAC
AGTTCCACTACTAGCGGGTTCCCACACAGTACAGCCATATGCGAACATAG
AATTATTCAAAGCGGCCTCGCCAACGCTGTCGAACGGCGGCAGCACGCCA
CACAGGCCAGCACCGGAACGCCCGCCAAAGCCAAAGTTCGGGGCCAAGCC
GACGAATGGACTGTATCCGCAAATAAGCCCGTCTAACGCGGGAAAGTCGG
AGGAAAGCCAAGACCAAGACTACGAGATATTGAGCCCAAGCGCGAGAATA
AGGCCAACGCGACCAGCGCCAAGGCCGCCATCCAACAAATCACCTCTCGT
TAATTCGGTGCCATCGTACGGAACACTGCCTGGCTCGTCGGATCTCGATG
GAGTTCCTTTTATTCTTAATCCGCTTCTCACTAGTGGCACGTCGCTTTCG
TTAAATAAGCAATTCCCTGTCATTAAAATCCGAACTCAGAAAGAACTGGA
TAAAGAGTATTCCTACGATTTTCGAACGGAGCGAGAAACTTGA
SEQ ID NO: 26
Organism: Li
SEQ ID NO: 25 nucleotides 556-655 (53)
TTCAAAGCGGCCTCGCCAACGCTGTCGAACGGCGGCAGCACGCCACACAG
GCCAGCACCGGAACGCCCGCCAAAGCCAAAGTTCGGGGCCAAGCCGACG
A
SEQ ID NO: 27
Organism: Li
Vps23
NADH-ubiquinone oxidoreductase,
20 Kd subunit (F)
A. mellifera XM_392437.4
ATGTTGCGTTCCCTTTTGACTAATCATACTTTCCAGCAAGGCTTGAATTT
AGTGGCGAGAAATAATGTCACTATTTTGGGAACTCAGAGTCCGCTGCTGG
AGCAGACAAAAAATTTAGCAACACAACCAGTTGAAAGCTCAGCACCAGCA
GCAACTCCAGCAAAAGAACCTTACAGCCCCTTTCAGAATAAGAGTAGCAT
GGTTGAATATGCACTGGCTAGACTTGACGATTTGTTGAATTGGGGCAGAA
AAGGTTCTATTTGGCCATTGACATTTGGTTTGGCTTGCTGTGCTGTGGAA
ATGATGCACATTGCTGCGCCACGTTATGATATGGACAGGTTTGGTGTTGT
GTTTCGTGCCTCGCCTAGGCATGCTGATGTAATTATTGTAGCTGGTACAG
TGACAAATAAGATGGCTCCTGCATTGAGGAAAGTTTACGATCAAATGCCA
GACCCCAAATGGGTGATTTCTATGGGCAGCTGTGCCAATGGTGGAGGCTA
CTATCATTATAGTTACTCTGTTGTAAGGGGCTGCGACAGGATCATACCTG
TGGACATTTATGTACCTGGCTGTCCTCCCACCGCAGAGGCACTCCTTTAT
GGGATTTTGCAGTTGCAGAAAAAAGTTAAGCGAATGGAGACTGCACAAGT
GTGGTATAGAAAATAA
SEQ ID NO: 28
Organism: Li
SEQ ID NO: 27 nucleotides 010-109 (46)
TCCCTTTTGACTAATCATACTTTCCAGCAAGGCTTGAATTTAGTGGCGAG
AAATAATGTCACTATTTTGGGAACTCAGAGTCCGCTGCTGGAGCAGACAA
SEQ ID NO: 29
Organism: Li
Vps28 Vacuolar protein sorting 28 (P)
A. mellifera XM_392314.4
ACTCAAGATCGTCCGGAACTTTATGAAGAAGTAAAATTATACAAGAATGC
CAGAGAACGAGAAAAGTATGAAAATCAAGCTGATTTGTATGCTGTTGTCA
ACACTTTGCAACATTTAGAGAAAGCTTACATCAGAGATTGTGTCACACCC
AAAGAGTACACTGCGGCGTGCAGCAAGTTGTTAGTTCAATACAGAGCAGC
TTTCAAACAGGTTCAAAGTGATCAGTTTCCAACAATTGATGCATTTGCCA
GAGCCTTCAAACTTGACTGTCCAGCAGCTCTTGAGAGAATCAAAGAGGAC
AGGCCAATTACAATTAAAGATGACAAGGGCAATACATCGAAGTGCATTGC
TGACATAGTTTCACTCTTCATTACCATCATGGATAAATTACGCTTGGAAA
TCAAAGCTATGGATCAACTTCACCCAGATCTCAGAGATTTAATGGATACC
ATGAACAGACTCAGTATACTGCCAAGTGACTTTGATGGAAAACAGAAGGT
TGCGGAGTGGCTTCAGACTTTGAATAATATGTCTGCTTCGGACGAACTGT
CAGATACGCAAGTTAGACAATTAATTTTTGATTTAGAAACGTCTTACAAT
GCATTTAACAAGGTTCTTCACAATTCTTAA
SEQ ID NO: 30
Organism: Li
SEQ ID NO: 29 nucleotides 412-511 (78)
GATCAACTTCACCCAGATCTCAGAGATTTAATGGATACCATGAACAGACT
CAGTATACTGCCAAGTGACTTTGATGGAAAACAGAAGGTTGCGGAGTGGC
SEQ ID NO: 31 + SEQ ID NO: 72
Organism: Li
Vps37/mod(r)
ESCRT Pathway (P)
A. mellifera XM_001122159.2
SEQ ID NO: 31
GAAAATGAAAATGTCGCCGTCAAGAGAAAACGACAGATTGACACGCTGAA
AATCTTTAATGATAATGTCTTGGAACTGCAAGAAGATGTTGAATATCAAG
TGCTGTTCAATGCTGGAGAAAGATGTATGGCAATTATGGTCTCATTGTCT
CCAGACTTTCCACTGGAAAAACCAGTGCTCCGAGTTTCACCATCAATAAA
GCATAAATGGTGTAATGAACACAGTGAAATTACTAGTGCACCAGGATTAT
TAAATTTTACGGTTCATAGTGACCTTGGGCGTGTTGTGCAAGCTATTATT
AGGGAGTTCAGTAAAAATCCACCACAACTGTTGGAGGAAAATTCTGCACT
CACAGATGTAGCTGGTAGAGTATCACCGTCGTACACATGGAAACCACACG
AGCTTGCT [gap]
SEQ ID NO: 72
AGTCAAGAGCAAAGCGAAGTGATAGCCCAGGACTTTCTTGATCGCAAAAT
TGACGTGGAGCGTTTTCTTAGTACGTACGTCGAGTGTCGGAAGCTCGGCC
AGGCCAGGCGGACCAAGGAGGAGAAACTCACACATCAACTCAACGAGCTA
AAG
SEQ ID NO: 32
Organism: Li
SEQ ID NO: 31 nucleotides 465-564 (67)
CGTGGAGCGTTTTCTTAGTACGTACGTCGAGTGTCGGAAGCTCGGCCAGG
CCAGGCGGACCAAGGAGGAGAAACTCACACATCAACTCAACGAGCTAAAG
SEQ ID NO: 33
Organism: Li
Vps37b
ESCRT Pathway (P)
A. mellifera XM_001120612.2
ATGTACAAGACATTTCAAGAGCCTGATATTCCAGCTGCTATAGGTCCAGT
TTCAAACCTTAGCACGAACGAATTAAAAGATCTACTGAATGACGAGGATA
AGTTTGAAGAAATCATCAAAACTAATCAACAGTTACTGGAGTTTGAGTCG
GAGAAAGAAGAAATAATGGTGAGGAATCGATCTTTAGCAGAATTCAATTT
ATCAAAAGAACCTGAACTAGAAGAAGCTAAAAATTTCATAAGGTCCCTCA
GCGAAGAAGGCAATCAATTGTGTTCCAGTGTTCAAGCCAAACTTGAACAA
ATAAAAAATAATGCTGGTGCAATGTCAACTGAAACT
SEQ ID NO: 34
Organism: Li
SEQ ID NO: 33 nucleotides 90-189 (60)
TGACGAGGATAAGTTTGAAGAAATCATCAAAACTAATCAACAGTTACTGG
AGTTTGAGTCGGAGAAAGAAGAAATAATGGTGAGGAATCGATCTTTAGCA
SEQ ID NO: 35
Organism: Li
Vps22/lsn
Regulation of Notch signaling pathway (F)
A. mellifera XM_003251158.1
ATGAGGAGAAAACCAGGAGTTGGAGCAATCCAAAAACAAAAATATGAACA
AGAACGGTATAGAGACAAAGGAACGGAGCTTCAAGAGAATCAATTCGAGC
AGATGACTAAGCAAATGGAAACTTTTAGAATAAATTTGGAAGAGTTTGCC
TCGAAGCATAAGAATGAAATAAAGAAAAATGCACAATTCAGGCGTCAGTT
TACAGAAATGTGTGCTTCTATAGGTGTAGATCCATTAGCATCTGGCAAAG
GTTTTTGGTCTGTATTAGGTATTGGAGATTTCTACTATGAACTTGGAATT
CAAATAGTTGAAGTATGTCTTGCAACTAATTACAAAAATGGAGGCTTGAT
ATCGCTGGATGAACTAAGAGAACGATTAATCCAAGCAAGAGGACGTCGCA
AAGAACATCAAGAGATAACAAACGAGGATTTACTTGCTTCAGCTAAAAAA
CTTAAAATATTAGGCAATGGATTCTCTGTGGTACCCATCAGTAAAGGCAA
ATACTTAGTTCAGTCTATACCTGGAGAGTTGAGCATGGATCACACTGCAG
TCTTGCAGCAAACAAATAACAACGGCAATGCCTTTATTTCAAAATCATTG
CTACAATCAGAATTAAAATGGGAGAGTGAAAGGGCTCAAAAAGCTCTTGA
TCATATGGTCAAAGAGGGATTAGCTTGGATTGATAATCAAAATGAAGGAG
AACCATTGTACTGGTTTCCAAGTTTGTTCACTGCTTGCATTGCTTCAAAA
ACGTAA
SEQ ID NO: 36
Organism: Li
SEQ ID NO: 35 nucleotides 520-619 (66)
CCTGGAGAGTTGAGCATGGATCACACTGCAGTCTTGCAGCAAACAAATAA
CAACGGCAATGCCTTTATTTCAAAATCATTGCTACAATCAGAATTAAAAT
SEQ ID NO: 37 + SEQ ID NO: 73
Organism: Li
Vps25
vesicle-mediated transport vacuolar
protein sorting 25 (P)
A. mellifera XM_395839.4
SEQ ID NO: 37
ATGACTGAAATTGATTGGCCCTGGCAATACAGTTTTCCTCCTTTTTTCAC
ACTTCAGCCGCATGCTGAAACAAGGGCAAAACAAATTGACGCATGGAAGA
GTTTAATTCTGGATTATTTTCGCACAACAAAGCAAGCAATTCTTGATGTT
CGTGAAGTTCACAGCAGCCCATTATTCAACAATAGTTCCATTGATCGAAA
ACTACCAGTAGAAGTTGTATCACTGATTCTTGAGGAGCTGTCAAAGTCAG
GTAATGCTACCCCACTAGACAAATCAAAGCAACGATGGGTTGTTTCATGG
CATACACTTGACGAATGGGCTGACATATTGTACTGTTGGGCACAA
[gap]
SEQ ID NO: 73
TTTTATGAACTAACCCAAGGAGAGGATACTGCTGATCAAGAATTTCATGG
CTTAGATAACGAAGTACTTGTGAGAGCTTTAAGAACATTAGAAGCATCTA
AAAAGGCTGAATTAATAATGTTTGATGATAATGAAGGTGTAAAGTTTTTC
TAA
SEQ ID NO: 38
Organism: Li
SEQ ID NO: 37 nucleotides 213-312 (68)
AGTTGTATCACTGATTCTTGAGGAGCTGTCAAAGTCAGGTAATGCTACCC
CACTAGACAAATCAAAGCAACGATGGGTTGTTTCATGGCATACACTTGAC
SEQ ID NO: 39 + SEQ ID NO: 74
Organism: Li
Vps36
vacuolar protein sorting (P)
A. mellifera XM_395542.3
SEQ ID NO: 39
ATGAACAGGTTCGAATACGCGGAGCCTCAGTTGTTGCCAAATGAAATTCA
CGTCAGACGCGATCGAGGCATCAGACTCTACGATGGAGACGTCAAGAGTC
CTTTCGAGGGTGGAGAGGTGACACTGACAAGCCACAGAATAATTTATAAA
ACGCAAGATGGTCTCACATTTGCCTTAAGACTTAGCTTAGTTGTATTTTT
TGAGGAGGAAAATCCAGGAGCTTTGTTTTTTACTCGCAGCAAGAAAGTAG
TGTTGCATTTAGCTGAGCCACCCAGTGATAAACTTACTGGACCAATTGAT
CATAGCCACTATAATTATGTTAAGTTGTCATTTAAAGAGGGACTGGATCC
TAATTTTGTTGCACACCTAAGTGATACAGTTATCAAAAAACTTTGGTAGA
TAGCGCCTATAAATGTTTCGTCAATACTCACAAGCTCTCCAAATGTGCAG
GGGAGTTCAAGTGCAAAACCACTACCTCAAATTAAACCCAGAACTGGTAT
TATAGGAATAGAAAGAAGTATACAAGAGCAACAGAAAGCCACAGACGAAA
GCATTACCGTTGCCTTTCAGGACTTGAAAAAGCTTATGGTCATGGCTAAG
GATATGGTTACAATATCCAAGACCATATCTGCAAAAATAAGGGAACGACA
AGGAGACATCACAGAGGATGAAACTGTTCGGTTCAAGTCATATTTGATGA
GTTTGGGTATCGATGATCCTGTGACCAGAGATGCCTACAGAACTGAGAAT
GAATATTTTAAACAGCTTGCTCGACAGGTAGCTGAAGTACTAGAAGAGCC
TGTAAAGGAAGTGGGTGGAATGATG [gap]
SEQ ID NO: 74
CAGGCAAGAGTTCACAGTGATTATGAAGTTGCCGAAGCTGTGTCACAATT
GATAAAAGACAGAGGATCAATTACAGCTACGGAATTAGCACAATCAGCAG
GAATTTCTGTGGTTTTGGCCTGTGAAAGGCTACTGATGACAGAGAAATAC
GGAAAAGCCTGCAGAGATGATTCGATCGAAGCATTGAGATTTTACCCTAA
TTTATTCCTGGAACAGGAGTCGTGA
SEQ ID NO: 40
Organism: Li
SEQ ID NO: 39 nucleotides 410-509 (57)
TAAATGTTTCGTCAATACTCACAAGCTCTCCAAATGTGCAGGGGAGTTCA
AGTGCAAAACCACTACCTCAAATTAAACCCAGAACTGGTATTATAGGAAT
SEQ ID NO: 41
Organism: Li
Vps2
protein transport (F)
A. mellifera XM_625161.3
ATGGAGTGGCTTTTTGGAAAACGCGTAACTCCTGAGGAGATGCTCAGGAA
GAATCAAAGAGCTTTGAACAAGGCAATGAGAGATCTAGACAGAGAAAGGA
TGAGAATGGAACAACAGGAAAAGAAAATCATAGCTGATATAAAGAAAATG
GCAAAAGATGGACAAATGGATGCTGTTAAAATCATGGCTAAAGATCTTGT
CAGGACAAGACGCTACGTTAAAAAGTTTATGTTAATGAAAGCAAATATTC
AAGCAGTATCTCTGAAAATTCAGACTTTACGCTCTCAAAACACAATGGCT
CAGGCCATGAAAGGAGTCACAAAGGCTATGCAAAATATGAACAAACAATT
AAATTTGCCACAAATTCAAAAGATATTGCAAGAGTTTGAAAAACAATCTG
AGATTATGGATATGAAAGAAGAAATAATGAATGATGCAATAGATGATGCC
ATGGAAGATGAGGGTGATGAAGAAGAAAGCGATGCGATTGTATCACAAGT
TCTGGATGAACTTGGCCTTCAATTAAACGACCAGTTGTCAGGACTACCTC
AAGCCTCAGGATCGTTGAGCATAGCAGGTTCGAAGCAACCAGTGGCCGCG
GCAGCTGGAGCCGGTAACGACGACGGCAATCTGGCGGATGCCGATGCGGA
TCTTCATGCGCGACTTGAAAATCTGCGTCGCGAGTAG
SEQ ID NO: 42
Organism: Li
SEQ ID NO: 41 nucleotides 548-649 (67)
CTCAAGCCTCAGGATCGTTGAGCATAGCAGGTTCGAAGCAACCAGTGGCC
GCGGCAGCTGGAGCCGGTAACGACGACGGCAATCTGGCGGATGCCGATGC
SEQ ID NO: 43
Organism: Li
Vps24
Charged multivesicular body protein 3 (P)
A. mellifera XM_394085.4
GGTTATCAGATTGACAGGCAGGTTAGAGCAATCCAAAGAGAAGAAGAAAA
AGTGAAAAAAACACTGAAAGAGGCAGCTAAAAAAGGTGACAAAGATGTC
TGTAAGATTTTGGCCAAAGAAGTGATTAGAGCGCAGAAAGCTTGTAAGAA
GTTGCATACCTCGAAAGCCCACCTGAATTCTGTCACCTTGCAAATGAAAA
ATCAATTAGCAACAATTAGAGTTGCTGGCTCTGTCTCAAAATCAACAGAA
GTCATGCAAGCTATGCAAGCGCTTATCAAAGTTCCAGAGGTCGCTGCAAC
AATGCGAGATTTGTCCAAAGAAATGATGAAGGCTGGTATTATTGAGGAAA
TGATGGATGAAACTATGGATTCTATGGAGGATTCTGAAGAAGTAGAGGAA
GCTGCTGATGAAGAAGTTGACAAGATTCTGTGGGAAGTTACTGCTGGACA
AATGGGAAGAGCACCAGACGTTGTCACAGAAACACCTGGAGCTTCAACTT
CAAAAGAAGAAGAAGAACCAGCTGAAGAACTGAGTGATGACAAGGAATTA
GAAGAAATGAAAAATAGATTACAAAGTCTTCGGAGTTAG
SEQ ID NO: 44
Organism: Li
SEQ ID NO: 43 nucleotides 87-186 (48)
TGACAAAGATGTCTGTAAGATTTTGGCCAAAGAAGTGATTAGAGCGCAGA
AAGCTTGTAAGAAGTTGCATACCTCGAAAGCCCACCTGAATTCTGTCACC
SEQ ID NO: 45
Organism: Li
Snf7/shrub
ESCRT-III pathway (F)
A. mellifera XM_395324.4
ATGAGTTTCTTTAGCAAGGTCTTCGGCGGGAAAAAGGAGCCGACCGCCCT
AACGACCGCCGAGGCGATACAAAAACTCCGAGAGACCGAGGAGATGCTCA
TCAAGAAACAGGACTTCCTTGAGACCAAGATCACACAGGAAATACAAACG
GCCAGGAAGAACGGTACCAAGAACAAACGAGCTGCCATTCAAGCGACTG
AAAAAAAGAAACGATATGAAAAGCAGCTACAACAAATCGATGGGACGTTG
TCCACAATTGAGATGCAAAGAGAAGCTCTCGAAAGTGCAAACACAAATAC
TGCTGTTCTTACCACAATGAAGAATGCAGCTGATGCTCTCAAAGCTGCAC
ATCAACACATGAATGTCGATGAAGTTCATGACATGATGGATGATATTGCT
GAGCAACAAGATGTTGCAAAGGAAATTTCAGATGCAATTTCAAACCCTGT
TGCATTTGGACAAGATATTGATGAAGATGAACTAGAGAAGGAGCTAGAAG
AATTAGAACAAGAAGAGTTAGACAAAGAATTGCTCGGTATACAAACCACA
GATGAACTACCTGCTGTTCCTGCTACAGCCTTACCAGCTGCACCCGAAAA
GAAATCAAAACCAAAACAAGAAGAAGATGATGATTTAAGAGAACTAGAGC
AATGGGCATCGTAA
SEQ ID NO: 46
Organism: Li
SEQ ID NO: 45 nucleotides 39-138 (66)
GCCGACCGCCCTAACGACCGCCGAGGCGATACAAAAACTCCGAGAGACCG
AGGAGATGCTCATCAAGAAACAGGACTTCCTTGAGACCAAGATCACACAG
SEQ ID NO: 47 [PCT SEQ ID NO: 103]
Organism: Om
Mor transcription coactivator
activity chromatin remodeling
A. mellifera XM_393008
ATAGCAGAATGTGAAGATGATGCTACTCACATAATCTATCCATCTGCTGA
TCCTCTAGAAGAAGAATACGCAAGGCCGTGTTTTCGAAGAGATCGATCTG
TTTTACTTCACTGGTATTATTTTCCAGATAGTTATGATTCGTGGGTCAAC
ATTGAACTTCCATGGGATTTTCCTGAAACTGCTCTTGGAAACCCTCCTCC
AAAGTCACCATATAAAGTTTCTGCTACTTGGGCACTAGATTTAGAACAAT
ACAATGAATGGATGAATGAGGAAGATTATGAAGTAGATGAAAGTGGTCAG
AAAAAAGTACATAAGTACAGGTTAAGCGTTGAAGACATGATGACACAATC
AGCACCACCTTCTGTTAAAAAGCAGAAAAGGAAGAGGTCTCCGACTCCAC
CACCAAAACTAGGCAAACGAAAAAGTGGGCGAGCTCCTGCTGGTCCTCAA
GGTATAAATTCTTCTACGGGTCCAAAGAAATCACGTGGAACTGGAGATGA
AGAAGAAGACTTAACTCAAGGCATGGATGATCCACCTGCAGAACCCAGGA
TAATGGAAGTCGTCTCTACAAATGCTAACACACCAAACTCAGCTCAAAAC
AGTACTGGTCCTATCATTTCCAGTAGCAAAAAACAGGATAATGAATTGCA
GCCATTAAAATCTGGAAATATGGCCGATCTTGATGAACCAGTGGATGGTG
AAAAAAGTAACTCACAAACGTCTCAAGACCGAGAAGAAAGGGATACGAGT
AAAGAAAGGAATGATGGTAGCAAAAGCGATGAACCAGAAGATAATGTTAC
AGAACAAACTCATCACATTGTTGTACCCAGTTACTCTGCGTGGTTTGATT
ACAATTCCATTCACACAATCGAGAAACGAGCTCTGTCTGAATTTTTCAAT
GGCAAAAACAAATCGAAAACACCAGAAATTTATTTGGCGTATAGAAATTT
CATGATCGACACTTATCGTTTGAACCCAACAGAGTACATCACGTCAACAG
CGTGCAGGCGAAATTTGGCTGGTGATGTTTGTGCGATAATGCGCGTACAT
GCTTTCCTCGAACAATGGGGTCTAATTAATTATCAGGTGGATGCCGATTC
AAGGCCGACGCCTATGGGTCCACCGCCAACCTCACACTTCCACGTGCTTT
CAGACACACCGTCAGGTTTAGCACCAGTTAATCCTAATCCTCCTAAAACG
CCACAGCCATCCGCAGCGAAAACTCTTTTGGATCTCGAAAAGAAGCCTGT
TATTACGGATGAAAAAGTTCCTCCGGTCGGACCCATGGCAAACTTTGGTC
TCAAGATCGATCAGTATTCGAAAAAACCAGCCGTACTGAAAAATAAACAA
GCTGCTGGTGCAACTCGTGATTGGACGGAACAAGAAACGTTATTATTACT
AGAGGCTTTAGAACTTCATAAGGACGATTGGAATAAGGTGTGCGAGCACG
TTGGCTCGAGGACACAAGATGAGTGCATTCTGCATTTCTTAAGGCTACCC
ATTGAGGATCCATACCTTGAGGAGCCGGAGGGTCTAGGCCCGTTGGCATA
TCAGCCTATTCCTTTCTCTAAGGCTGGAAATCCTGTTATGAGTACAGTAG
CTTTCCTGGCATCAGTGGTTGATCCAAGAGTAGCGGCAAGTGCGGCCAAA
GCTGCAATGGAAGAATTCGCTGCGATAAAGGATCAGGTGCCAGCAGCACT
TCTGGATCAACATTTAAGGAATGTGCAGGCTAGCGCGAATTCGGATGGTA
AATTCGATCCGGCAGCTGGTCTTGCACAGTCGGGTATCGCCGGAACCGGT
SEQ ID NO: 48 [PCT SEQ ID NO: 130]
Organism: Om
SEQ ID NO: 47 nucleotides 213-312 (75)
ATCGACACTTATCGTTTGAACCCAACAGAGTACATCACGTCAACAGCGTG
CAGGCGAAATTTGGCTGGTGATGTTTGTGCGATAATGCGCGTACATGCTT
SEQ ID NO: 49 [PCT SEQ ID NO: 104]
Organism: Li
Mor transcription coactivator
activity chromatin remodeling
A. mellifera XM_393008
CAGTACAATGAGTGGATGAATGAAGAAGATTATGAAGTAGATGATAGTGG
ACAGAAAAAGGTACATAAATATAGACTATCAGTTGAGGACTTGATGGCTC
CAACACCTGCATCAGGTAAAAAACAAAAGAGAAAAAGTCGCCCAGTCCTC
CTCCAAAACTTGGAAAAAGAAAAGTGGCAGAGCTCCTGGAGGAACTCAAG
GTGGGTTGTCCGGCCCAAAAAATCACGAGGTGGAGACGAAGAAGAAGACT
TGACACAAGGAATGGAGGATCCTCCGTCTGAGCCGAGAATAACGGAAGTT
GTAAATTCGAATACGAATGCATCTATCTCTGGACAGAATAGCAGCTCAGG
CATGGTGTCCAGCAAAAAACAGGACAATGACATGCAGCCACTCAAGTCTG
GAAACATGGCCGATTTGGATGAACCAGTTGATGGTGATAAAAGCAATTCG
CAAAATTCACAAGACAGAGAAGAACGTGACACGAGCAAGGAAAGAGGCGA
CGGCAGCAAGAGCGATGAGCCCGAGGATAACGTGACCGAGCAGACTCATC
ACATTGTGATTCCGAGCTACTCGGCGTGGTTTGACTACAACTCTATTCAC
ATGATTGAGAAGCGAGCACTATCAGAGTTTTTCAACGGCAAGAACAAGTC
TAAGACACCAGAAATCTACCTCGCTTACAGGAATTTCATGATCGACACCT
ATCGCCTCAATCCGACCGAGTACATTACTTCCACAGCCTGTAGGCGAAAC
TTAGCTGGTGATGTATGCGCTATCATGCGCGTGCACGCTTTTCTCGAACA
GTGGGGTCTGATCAATTATCAAGTGGATGCCGATTCAAGACCGACACCTA
TGGGACCTCCACCTACTTCGCACTTCCATGTTTTGTCAGATACACCGTCT
GGGTTAGCTCCAGTTAATCCAAACCCTCCCAAAACACCGCAGCCGTCAGC
GGCAAAGACGTTACTTGATCTCGAAAAGAAACCGATTATTGACGAGAAGA
TTCCAGCTGCTGGAGCGATGGCCAACTTCGGCCTGAAGCTCGATCAATAC
GCGAGGAAGCCTGCGGTTTTGAAAAACAAGCAAGCTGCTGGTGCTACTCG
CGAGTGGACGGAACAAGAAACGCTGTTATTGCTTGAAGCTTTGGAGTTAC
ACAAGGATGACTGGAATAAGGTTTGTGAGCATGTTGGTTCAAGAACTCAG
GATGAATGTATCCTACACTTTTTGCGATTACCCATTGAAGACCCGTACCT
TGAAGAACCAGAAGGTCTTGGTCCACTGGCATATCAGCCCATACCTTTTT
CTAAGGCTGGTAACCCTGTCATGAGCACCGTTGCATTTTTGGCTTCGGTA
GTGGATCCTAGAGTAGCAGCTAGTGCGGCAAAGGCTGCTATGGAAGAGTT
TGCTGCCATCAAAGACCAGGTGCCTGCTGCTTTACTGGATCAACATTTGA
GAAATGTTCAGGCTACTGCTGCTGATGGAAAATTCGACCCAGCCGCCGGT
CTTGCACAGTCAGGTATCGCAGGCACAGGA
SEQ ID NO: 50 [PCT SEQ ID NO: 127]
Organism: Li
SEQ ID NO: 49 nucleotides 492-591 (72)
AGAGGCGACGGCAGCAAGAGCGATGAGCCCGAGGATAACGTGACCGAGC
AGACTCATCACATTGTGATTCCGAGCTACTCGGCGTGGTTTGACTACAAC
T
SEQ ID NO: 51 [PCT SEQ ID NO: 120]
Organism: Om
TIF
Eukaryotic translation initiation
factor 3 subunit I-like (TIF)
translational initiation
A. mellifera XM_392780
ATGAAACCACTGATGTTACATGGGCACGAGCGTGCCATCACAAAAATCAA
ATACAACAGAGAAGGAGACTTACTTTTTTCTGCTAGTAAAGATAAACAAC
CGAATGTTTGGTATTCGTTAAACGGAGAAAGACTTGGAACATTCAATGGA
CACAATGGTTCAGTTTGGTGTATTGATGTCAATTGGGATACAACACGCTT
TTTATCCGGTAGTGGTGACAACACTTTAAGATTATGGGATTGTGCAACAG
GAAAAGAGATTAGTCAATTGTCCACAAACAGTTCAGTAAGAGCTTGTGCA
TTTAGCTATTCAGGCAATCTTGCTGTATATGCCACTGACAAAGCTCTTGG
ACACCAATGTGAAATGTTTATTATTGACATTAGGTCTCCTGAAAGTGTTC
TTTCTCAAGATGATAACGTTTGTAGAACTTCAGTCAGTGGTTCAAGGATT
TCATCTCTCTTGTGGGGAGCTCTCGATGAATCTATTATTACTGGTCATGA
AAATGGTGATTTAAACATCTGGGACAGTAGGACTGGAAAGAAATTGAGTG
ATGCTCAGGGTCACAAGGGTCAAATTAATGACATGCAGTTCAACAAGGAT
GGAACTATGTTCGTTACAGCCTCAAAGGACCACACTGCAAAGTTGTTTGA
CAGTGAATCTCTTGTTCCATTGAAAACGTATAAAACAGAAAGGCCAGTTA
ACTCTGCCACGATATCTCCAATCTTTGATCACGTCGTAGTTGGAGGTGGT
CAAGACGCTATGGACGTCACCACGACATCGACGAAACAAGGAAAATTCGA
TGCTCGTTTTTTCCATCTTGTCTTTGAAGAAGAATTTGCACGTTTAAAAG
GTCACTTTGGTCCAATTAACTCGCTGGCCTTTCATCCAAATGGACGCAGT
CTGTCTACTGGAGGAGAGGATGGTTATATTCGTATAAATACATTTGATCA
GTCCTATTTCGATTTTCATTTTGAGTACTAA
SEQ ID NO: 52 [PCT SEQ ID NO: 131]
Organism: Om
SEQ ID NO: 51 nucleotides 375-474
with C472G change to eliminate SacI
site in target sequence (61)
TGACATTAGGTCTCCTGAAAGTGTTCTTTCTCAAGATGATAACGTTTGTA
GAACTTCAGTCAGTGGTTCAAGGATTTCATCTCTCTTGTGGGGAGCTGTC
SEQ ID NO: 53 [PCT SEQ ID NO: 121]
Organism: Li
TIF
Eukaryotic translation initiation
factor 3 subunit I-like (TIF)
translational initiation
A. mellifera XM_392780
ATGAAACCTTTAATGTTGCACGGGCACGAGCGTGCCATTACAAAAATTAA
GTATAACAGAGAAGGAGATTTACTGTTTTCAGCCAGTAAAGACAAACAGC
CTAATGTATGGTACTCTCTCAATGGTGAAAGATTGGGTACATTCAATGGC
CACAACGGTTCAGTTTGGTGCATTGACGTCAATTGGGATACTACACGCTT
CCTTTCTGGCAGTGGTGACAACACACTGAGAATATGGGATTGTCAAACAG
GCAAAGAAATAAGCCAGTTGTCAACCAACAGTTCTGTAAGAGCTTGCGCT
TTCAGTTACTCTGGTAACCTTGCTGTTTATGCAACTGACAAAGCTCTTGG
TCATCAGTGTGAAATGTTCATTATTGATATTAGGACTCCTGAAAGTGTAC
TTTCTCAGGAAGATAATGTCTGCAGAACTATGATCGGTGGTTCCAGGATC
TCATCTCTCCTTTGGGGAGCTCTTGATGAAACCATCATAACTGGTCATGA
GAATGGTGACTTGACCATTTGGGATAGTAGGACTGGAAAGAAATTGAGCG
ACGCTCAGGGACACAAAGGTCAAATAAATGATATGCAGTTCAACAAAGAT
GCCACTATGTTTTGCACTGCCTCCAAAGATCACACTGCTAAATTATTTGA
CAGCGAATCTCTAGTTTCACTAAAAACTTACAAAACTGAGCGGCCCGTTA
ACTCGGCTACGATTTCTCCAATCTTTGATCATGTCGTTGTTGGAGGTGGT
CAAGATGCCATGGATGTAACAACGACGTCGACGAAACAAGGAAAATTCGA
CGCTCGTTTTTATCATCTCGTCTTTGAAGAAGAATTTGCACGTTTAAAGG
GCCATTTCGGCCCCATCAACTCATTATCCTTCCACCCGAACGGCAGAAGC
TTAGCCACTGGAGGAGAGGACGGTTACATTCGTATCAAC
SEQ ID NO: 54 [PCT SEQ ID NO: 128]
Organism: Li
SEQ ID NO: 53 nucleotides 840-939 (70)
ACGTTTAAAGGGCCATTTCGGCCCCATCAACTCATTATCCTTCCACCCGA
ACGGCAGAAGCTTAGCCACTGGAGGAGAGGACGGTTACATTCGTATCAAC
SEQ ID NO: 55
Silencing Construction #1,
Om Triple Gene Construct
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
ATCGACACTTATCGTTTGAACCCAACAGAGTACATCACGTCAACAGCGTG
CAGGCGAAATTTGGCTGGTGATGTTTGTGCGATAATGCGCGTACATGCTT
TGACATTAGGTCTCCTGAAAGTGTTCTTTCTCAAGATGATAACGTTTGTA
GAACTTCAGTCAGTGGTTCAAGGATTTCATCTCTCTTGTGGGGAGCTGTC
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCGGACAGCTCCCCACAAGAGAGATGAAATCCTTGAACCACTGACTG
AAGTTCTACAAACGTTATCATCTTGAGAAAGAACACTTTCAGGAGACCTA
ATGTCAAAGCATGTACGCGCATTATCGCACAAACATCACCAGCCAAATTT
CGCCTGCACGCTGTTGACGTGATGTACTCTGTTGGGTTCAAACGATAAGT
GTCGATTGAGCATCTCCTCCGTCTCGCGTAGTTTCTGTATGGCCTCCGAG
GTCGTCGGAGCAGCCGCCTCCTTCTTCCCGCCGAAGACCTTCGTGAAGAA
GCTCAT
SEQ ID NO: 56
Silencing Construct #2,
Li Triple Gene Construct
GCCGACCGCCCTAACGACCGCCGAGGCGATACAAAAACTCCGAGAGACCG
AGGAGATGCTCATCAAGAAACAGGACTTCCTTGAGACCAAGATCACACAG
AGAGGCGACGGCAGCAAGAGCGATGAGCCCGAGGATAACGTGACCGAGC
AGACTCATCACATTGTGATTCCGAGCTACTCGGCGTGGTTTGACTACAAC
TACGTTTAAAGGGCCATTTCGGCCCCATCAACTCATTATCCTTCCACCCG
AACGGCAGAAGCTTAGCCACTGGAGGAGAGGACGGTTACATTCGTATCAA
CGGCTCGAACGAGCCGACTAATTGTCTTTAAACGCGCGATATAAGCGCAC
AATGCTCGAGAAACGATAAACTCTATCGCTCTGTCGCGTGCGTGGCATCT
TCGCGCGGTTGATACGAATGTAACCGTCCTCTCCTCCAGTGGCTAAGCTT
CTGCCGTTCGGGTGGAAGGATAATGAGTTGATGGGGCCGAAATGGCCCTT
TAAACGTAGTTGTAGTCAAACCACGCCGAGTAGCTCGGAATCACAATGTG
ATGAGTCTGCTCGGTCACGTTATCCTCGGGCTCATCGCTCTTGCTGCCGT
CGCCTCTCTGTGTGATCTTGGTCTCAAGGAAGTCCTGTTTCTTGATGAGC
ATCTCCTCGGTCTCTCGGAGTTTTTGTATCGCCTCGGCGGTCGTTAGGGC
GGTCGGC
SEQ ID NO: 57
P1 - CaMV 35S Promoter & Omega UTR
AGATTAGCCTTTTCAATTTCAGAAAGAATGCTAACCCACAGATGGTTAGA
GAGGCTTACGCAGCAGGTCTCATCAAGACGATCTACCCGAGCAATAATCT
CCAGGAAATCAAATACCTTCCCAAGAAGGTTAAAGATGCAGTCAAAAGAT
TCAGGACTAACTGCATCAAGAACACAGAGAAAGATATATTTCTCAAGATC
AGAAGTACTATTCCAGTATGGACGATTCAAGGCTTGCTTCACAAACCAAG
GCAAGTAATAGAGATTGGAGTCTCTAAAAAGGTAGTTCCCACTGAATCAA
AGGCCATGGAGTCAAAGATTCAAATAGAGGACCTAACAGAACTCGCCGTA
AAGACTGGCGAACAGTTCATACAGAGTCTCTTACGACTCAATGACAAGAA
GAAAATCTTCGTCAACATGGTGGAGCACGACACACTTGTCTACTCCAAAA
ATATCAAAGATACAGTCTCAGAAGACCAAAGGGCAATTGAGACTTTTCAA
CAAAGGGTAATATCCGGAAACCTCCTCGGATTCCATTGCCCAGCTATCTG
TCACTTTATTGTGAAGATAGTGGAAAAGGAAGGTGGCTCCTACAAATGCC
ATCATTGCGATAAAGGAAAGGCCATCGTTGAAGATGCCTCTGCCGACAGT
GGTCCCAAAGATGGACCCCCACCCACGAGGAGCATCGTGGAAAAAGAAGA
CGTTCCAACCACGTCTTCAAAGCAAGTGGATTGATGTGATATCTCCACTG
ACGTAAGGGATGACGCACAATCCCACTATCCTTCGCAAGACCCTTCCTCT
ATATAAGGAAGTTCATTTCATTTGGAGAGAACACGGGGGACTCTAGATAT
TTTTACAACAATTACCAACAACAACAAACAACAAACAACATTACAATTAC
TATTTACAATTACA
SEQ ID NO: 58
sgFIMV Promoter
TTTACAGTAAGAACTGATAACAAAAATTTTACTTATTTCCTTAGAATTAA
TCTTAAAGGTGATAGTAAACAAGGACGATTAGTCCGTTGGCAAAATTGGT
TCAGCAAGTATCAATTTGATGTCGAACATCTTGAAGGTGTAAAAAACGTT
TTAGCAGATTGCCTCACGAGAGATTTTAATGCTTAAAAACGTAAGCGCTG
ACGTATGATTTCAAAAAACGCAGCTATAAAAGAAGCCCTCCAGCTTCAAA
GTTTTCATCAACACAAATTCTAAAAACAAAATTTTTTAGAGAGGGGGAGT
G
SEQ ID NO: 59
AtActin7 Terminator including 3UTR
GTGTGTCTTGTCTTATCTGGTTCGTGGTGGTGAGTTTGTTACAAAAAAAT
CTATTTTCCCTAGTTGAGATGGGAATTGAACTATCTGTTGTTATGTGGAT
TTTATTTTCTTTTTTCTCTTTAGAACCTTATGGTTGTGTCAAGAAGTCTT
GTGTACTTTAGTTTTATATCTCTGTTTTATCTCTTCTATTTTCTTTAGGA
TGCTTGTGATGATGCTGTTTTTTTTTGTCCCTAAGCAAAAAAATATCATA
TTATATTTGGTCCTTGGTTCATTTTTTTGGTTTTTTTTTGTCTTCACATA
TAAATATTGTTTGAATGTCTTCAATCTTTTATTTGTATGAGACAATTATT
TAAGTATCGGGTGACAATGCAGCTATTATGTATTGTCGATTGTTATATTG
GCGCCCAAAATATATACTTAGCCTAAGAATTTGGTAAGTGAGTGGCTTAT
GTTTTACTCCAGCAAAAATTGTGTGTGTATTACCATTCTGATGCGAAACA
AGAAAAGAATTTGATCTAAGAAACCAAGTTTATTCACTAGTTAAAAAACA
AATGACCTAATGTAATCGACTCCACATATCAAAATACGTAAAACAAACAT
TGTATGTTGACAAAAGGGAAAAGAAATGATTTATTTGGTTAAAAAGAAAG
CTGGATTCAATTGCAACAGTTTAGTCGAAATCATTTTGAAAGGCTTACAA
TGGATTGAATGTGAATATTCCATTAAGCCGCTTCTGTCTACACAGAATGT
TACGCTTGGAGAGCAGCAATCATTTTCACGTTTTTATCTTTTTAGGTGGA
CATGTATATTATTGGTTACGCCTTTGGAGTTTTTCGAAATTTATTTCTTT
CAAATCACAAGATGACTAAACATCACAATCTGTTTATCTTCCTAACTAGT
TAAATTTTTGTCCCCACCATT
SEQ ID NO: 60
NOS Terminator
GATCGTTCAAACATTTGGCAATAAAGTTTCTTAAGATTGAATCCTGTTGC
CGGTCTTGCGATGATTATCATATAATTTCTGTTGAATTACGTTAAGCATG
TAATAATTAACATGTAATGCATGACGTTATTTATGAGATGGGTTTTTATG
ATTAGAGTCCCGCAATTATACATTTAATACGCGATAGAAAACAAAATATA
GCGCGCAAACTAGGATAAATTATCGCGCGCGGTGTCATCTATGTTACTAG
ATC
SEQ ID NO: 61
Loop Sequence
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCG
SEQ ID NO: 62
Sense transcript construct #1
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
ATCGACACTTATCGTTTGAACCCAACAGAGTACATCACGTCAACAGCGTG
CAGGCGAAATTTGGCTGGTGATGTTTGTGCGATAATGCGCGTACATGCTT
TGACATTAGGTCTCCTGAAAGTGTTCTTTCTCAAGATGATAACGTTTGTA
GAACTTCAGTCAGTGGTTCAAGGATTTCATCTCTCTTGTGGGGAGCTGTC
SEQ ID NO: 63
Sense transcript construct #2
GCCGACCGCCCTAACGACCGCCGAGGCGATACAAAAACTCCGAGAGACCG
AGGAGATGCTCATCAAGAAACAGGACTTCCTTGAGACCAAGATCACACAG
AGAGGCGACGGCAGCAAGAGCGATGAGCCCGAGGATAACGTGACCGAGC
AGACTCATCACATTGTGATTCCGAGCTACTCGGCGTGGTTTGACTACAAC
TACGTTTAAAGGGCCATTTCGGCCCCATCAACTCATTATCCTTCCACCCG
AACGGCAGAAGCTTAGCCACTGGAGGAGAGGACGGTTACATTCGTATCAA
C
SEQ ID NO: 64
hpRNA construct #3, Om Single
gene construct
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCGTGAGCATCTCCTCCGTCTCGCGTAGTTTCTGTATGGCCTCCGAG
GTCGTCGGAGCAGCCGCCTCCTTCTTCCCGCCGAAGACCTTCGTGAAGAA
GCTCAT
SEQ ID NO: 65
hpRNA construct #4, Om Double
gene construct
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
AATGTATTGCTGACATTGTTTCACTCTTCATAACATTAATGGATAAGTTA
CGACTTGAAATTAAAGCTATGGATCAACTTCATCCAGATCTTAGAGATTT
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCGAAATCTCTAAGATCTGGATGAAGTTGATCCATAGCTTTAATTTC
AAGTCGTAACTTATCCATTAATGTTATGAAGAGTGAAACAATGTCAGCAA
TACATTTGAGCATCTCCTCCGTCTCGCGTAGTTTCTGTATGGCCTCCGAG
GTCGTCGGAGCAGCCGCCTCCTTCTTCCCGCCGAAGACCTTCGTGAAGAA
GCTCAT
SEQ ID NO: 66
Sense transcript construct
#4, Om Double gene construct
ATGAGCTTCTTCACGAAGGTCTTCGGCGGGAAGAAGGAGGCGGCTGCTCC
GACGACCTCGGAGGCCATACAGAAACTACGCGAGACGGAGGAGATGCTCA
AATGTATTGCTGACATTGTTTCACTCTTCATAACATTAATGGATAAGTTA
CGACTTGAAATTAAAGCTATGGATCAACTTCATCCAGATCTTAGAGATTT
SEQ ID NO: 67
hpRNA construct #5, Li
Single gene construct
GCCGACCGCCCTAACGACCGCCGAGGCGATACAAAAACTCCGAGAGACCG
AGGAGATGCTCATCAAGAAACAGGACTTCCTTGAGACCAAGATCACACAG
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCGCTGTGTGATCTTGGTCTCAAGGAAGTCCTGTTTCTTGATGAGCA
TCTCCTCGGTCTCTCGGAGTTTTTGTATCGCCTCGGCGGTCGTTAGGGCG
GTCGGC
SEQ ID NO: 68
hpRNA construct #6, Li
Double gene construct
GCCGACCGCCCTAACGACCGCCGAGGCGATACAAAAACTCCGAGAGACCG
AGGAGATGCTCATCAAGAAACAGGACTTCCTTGAGACCAAGATCACACAG
GATCAACTTCACCCAGATCTCAGAGATTTAATGGATACCATGAACAGACT
CAGTATACTGCCAAGTGACTTTGATGGAAAACAGAAGGTTGCGGAGTGGC
GGCTCGAACGAGCCGACTAATTGTCTTTAAACGCGCGATATAAGCGCACA
ATGCTCGAGAAACGATAAACTCTATCGCTCTGTCGCGTGCGTGGCATCTT
CGCGCGGCCACTCCGCAACCTTCTGTTTTCCATCAAAGTCACTTGGCAGT
ATACTGAGTCTGTTCATGGTATCCATTAAATCTCTGAGATCTGGGTGAAG
TTGATCCTGTGTGATCTTGGTCTCAAGGAAGTCCTGTTTCTTGATGAGCA
TCTCCTCGGTCTCTCGGAGTTTTTGTATCGCCTCGGCGGTCGTTAGGGCG
GTCGGC
SEQ ID NO: 69
Sense transcript construct
#6, Li Double gene construct
GCCGACCGCCCTAACGACCGCCGAGGCGATACAAAAACTCCGAGAGACCG
AGGAGATGCTCATCAAGAAACAGGACTTCCTTGAGACCAAGATCACACAG
GATCAACTTCACCCAGATCTCAGAGATTTAATGGATACCATGAACAGACT
CAGTATACTGCCAAGTGACTTTGATGGAAAACAGAAGGTTGCGGAGTGGC

1. An isolated double stranded ribonucleic acid molecule (dsRNA) comprising a unit of a first strand of nucleotides that is substantially identical to at least 17 contiguous nucleotides set forth in SEQ ID NO: 1-46 and 70-74 and a second strand of nucleotides that is substantially complementary to said first strand of nucleotides.

2. The isolated dsRNA according to claim 1 wherein said first strand of nucleotides is substantially identical to at least 17 contiguous nucleotides set forth in SEQ ID NO: 1-46 and 70-74.

3. The dsRNA according to claim 1 wherein said first and second strands of nucleotides are at least about 25, 35, 50, 70 or 100 nucleotides in length.

4. The dsRNA according to claim 1 wherein over their respective lengths said first and second strands of nucleotides are 70-100% identical to SEQ ID NO: 1-46 and 70-74.

5. The dsRNA according to claim 1 wherein the sequences of said first and second strands of nucleotides are less than about 80% identical to the sequence of the honey bee ortholog of said first and second strands of nucleotides.

6. The dsRNA according to claim 1 comprising at least two (2) of said units.

7. The dsRNA according to claim 6 wherein said at least two units are derived from different sequences selected from the group consisting of SEQ ID NO: 1-46 and 70-74.

8. The dsRNA according to claim 1 further comprising a loop region separating said first strand and said second strand nucleotides.

9. A vector comprising an expression control sequence operatively linked to a nucleotide sequence that is a template for one or both strands of claim 1.

10. A host cell comprising the expression vector according to claim 9.

11. The host cell according claim 10 wherein said host is a bacterial cell or a yeast cell.

12. The host cell according to claim 11 wherein said host is an Agrobacterium.

13. A plant tissue transformed with the host cell according to claim 12.

14. A plant tissue comprising the dsRNA or the vector according to claim 9.

15. An isolated nucleic acid comprising a sequence that selectively hybridizes under high stringency hybridization conditions to a sequence selected from the group consisting of SEQ ID NO: 1-46 and 70-74 and complementary sequences thereof.

16. The isolated nucleic acid according to claim 15 wherein said nucleic acid is 90-99.99 percent identical to said sequence selected from the group consisting of SEQ ID 1-46 and 70-74 and complementary sequences thereof.

17. The isolated nucleic acid of claim 15 wherein said nucleic acid comprises at least 17 contiguous nucleotides of a sequence selected from the group consisting of SEQ ID NO: 1-46 and 70-74 and complementary sequences thereof.

18. The isolated nucleic acid according to claim 17 wherein said nucleic acid comprises at least 25 contiguous nucleotides of sequence selected from the group consisting of SEQ ID NO: 1-46 and 70-74 and complementary sequences thereof.

19. The isolated nucleic acid according to claim 15 wherein said nucleic acid is less than about 80% identical to the honey bee ortholog of said nucleic acid.

20. A vector comprising the isolated nucleic acid according to any claim 15 operably linked to an expression control sequence.

21. A host cell comprising the vector according to claim 20.

22. A plant tissue comprising the vector according to claim 20.

23. The plant tissue according to claim 22 wherein said tissue is selected from the group consisting of leaf tissue, veins, phloem, xylem, petioles, small branches, branches, flowers, trunk, fruits and seeds.

24. An isolated small inhibitory ribonucleic acid molecule (siRNA) that inhibits expression of an L. invasa or O. maskelli nucleic acid molecule encoding Multivesticular body subunit 12B-like (Dmel), NADH dehydrogenase [ubiquinone] iron sulfur protein 7 (Vps23), Vacuolar Protein Sorting-Associated Protein 28 homolog (Vps28), Vacuolar protein sorting associated protein 37A like (Vps37/mod-r), Vacuolar protein sorting associated protein 37B like (Vps37b). Vacuolar sorting protein SNF8 like (Vps22/Isn), Vacuolar protein sorting associated protein 25 like (Vps 25), Vacuolar protein sorting associated protein 36 (Vps36), Charged Multivesicular Body Protein 2a like (Vps2), Charged multivesicular body protein 6 like (Vps20), Charged multivesicular body protein 3 like (Vps24), or Charged Multivesicular Body Protein 4b like (Snf7/shrub).

25. A method of producing a pest resistant plant comprising expressing a dsRNA according to claim 1 in said plant or propagating reproductive material of said plant.

26. The method according to claim 25 wherein said plant is Eucalyptus.

27. The method according to claim 25 wherein said pest is a gall wasp.

28. The method according to claim 27 wherein said pest is L. invasa or O. maskelli.

29. A method of inhibiting a pest infestation comprising cultivating a plant comprising a dsRNA according to claim 1 to inhibit said infestation.

30. The method according to claim 29 wherein said plant is Eucalyptus.

31. The method according to claim 30 wherein said pest is L. invasa or O. maskelli.

32. A method of producing a plant resistant to a plant pathogenic pest comprising:
(a) transforming a plant cell with a recombinant DNA construct or combination of constructs that express the dsRNA according to claim 1;
(b) regenerating a plant from the transformed plant cell; and
(c) growing the transformed plant cell under conditions suitable for the transcription said recombinant DNA construct,
said grown transformed plant thus being resistant to said pest compared to an untransformed plant.

33. The method according to claim 32 further comprising transforming said plant cell with a recombinant DNA construct that expresses a single stranded RNA that is complementary to one strand said dsRNA or a fragment thereof.

34. The method according to claim 32 wherein said plant is Eucalyptus.

35. The method according to claim 34 wherein said pest is L. invasa or O. maskelli.
